(19) United States
(12) Patent Application Publication
(10) Pub. No.: US 2018/0364252 A1
(43) Pub. Date: Dec. 20, 2018

(54) COMPOSITIONS, DEVICES, AND METHODS OF PSORIASIS FOOD SENSITIVITY TESTING

(71) Applicant: Biomerica, Inc., Irvine, CA (US)

(72) Inventors: Zackary IRANI-COHEN, Irvine, CA (US); Elisabeth LADERMAN, Irvine, CA (US)

(21) Appl. No.: 16/013,774

(22) Filed: Jun. 20, 2018

Related U.S. Application Data
(63) Continuation of application No. PCT/US2016/068136, filed on Dec. 12, 2016.
(60) Provisional application No. 62/270,578, filed on Dec. 21, 2015.

(51) Int. Cl. G01N 33/68

(52) U.S. Cl. CPC G01N 33/6854; G01N 2800/205

(57) ABSTRACT

Contemplated test kits and methods for food sensitivity are based on rational-based selection of food preparations with established discriminatory p-value. Exemplary kits include those with a minimum number of food preparations that have an average discriminatory p-value of ≤0.07 as determined by their raw p-value or an average discriminatory p-value of ≤0.10 as determined by FDR multiplicity adjusted p-value. In further contemplated aspects, compositions and methods for food sensitivity are also stratified by gender to further enhance predictive value.

### RELATED APPLICATIONS

This application is a continuation of International Application No. PCT/US2016/068136, filed Dec. 21, 2016, which claims priority to U.S. Provisional Patent Application No. 62/270,578, filed Dec. 21, 2015. Each of the foregoing applications is incorporated herein by reference in its entirety.

### FIELD

The field of the subject matter disclosed herein is sensitivity testing for food intolerance, and especially as it relates to testing and possible elimination of selected food items as foods that exacerbate or worsen symptoms or foods that, when removed, alleviate symptoms in patients diagnosed with or suspected to have psoriasis.

### BACKGROUND

The background description includes information that may be useful in understanding the present disclosure. It is not an admission that any of the information provided herein is prior art or relevant to the appended claims, or that any publication specifically or implicitly referenced is prior art.

Food sensitivity (also known as food intolerance), especially as it relates to psoriasis (a type of autoimmune disease), often presents with skin lesions, scaly patches, papules, and plaques that usually itch. The underlying causes of psoriasis are not well understood in the medical community. Psoriasis may be visually diagnosed, along with various tests to exclude various other inflammatory or infectious conditions. Unfortunately, treatment of psoriasis may often be less than effective and may present new difficulties due to immune suppressive or modulatory effects. In certain instances, elimination of other one or more food items has also shown promise in at least reducing incidence and/or severity of the symptoms. However, psoriasis is often quite diverse with respect to dietary items triggering symptoms, whereby no standardized test to help identify trigger food items with a reasonable degree of certainty is known, thus leaving such patients often to trial-and-error.

While there are some commercially available tests and labs to help identify trigger foods, the quality of the test results from these labs is generally poor as is reported by a consumer advocacy group (e.g., http://www.which.co.uk/news/2008/08/food-allergy-tests-could-risk-your-health-154711/). Most notably, problems associated with these tests and labs were high false positive rates, high intra-patient variability, and inter-laboratory variability, rendering such tests nearly useless. Similarly, further inconclusive and highly variable test results were also reported elsewhere (Alternative Medicine Review, Vol. 9, No. 2, 2004: pp 198-207), and the authors concluded that this may be due to food reactions and food sensitivities occurring via a number of different mechanisms. For example, not all psoriasis patients show positive response to food A, and not all psoriasis patients show negative response to food B. Thus, even if a psoriasis patient shows positive response to food A, removal of food A from the patient's diet may not relieve the patient's psoriasis symptoms. In other words, it is not well determined whether food allergens used in the currently available tests are properly selected based on high probabilities of correlating sensitivities to those food allergens to psoriasis.

All publications identified herein are incorporated by reference to the same extent as if each individual publication or patent application was specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, even though various tests for food sensitivities are known in the art, all or almost all of them suffer from one or more disadvantages. Therefore, there is still a need for improved compositions, devices, and methods of food sensitivity testing, especially for identification and possible elimination of trigger foods for patients identified with or suspected of having psoriasis.

### SUMMARY

The subject matter described herein provides systems and methods for testing food intolerance in patients diagnosed with or suspected to have psoriasis. One aspect of the disclosure is a test kit with for testing food intolerance in patients diagnosed with or suspected to have psoriasis. The test kit includes a plurality of distinct food preparations coupled to individually addressable respective solid carriers. The plurality of distinct food preparations have an average discriminatory p-value of ≤0.07 as determined by raw p-value or an average discriminatory p-value of ≤0.10 as determined by FDR multiplicity adjusted p-value.

Another aspect of the embodiments described herein includes a method of testing food intolerance in patients diagnosed with or suspected to have psoriasis. The method includes a step of contacting a food preparation with a bodily fluid of a patient that is diagnosed with or suspected to have psoriasis. The bodily fluid is associated with gender identification. In certain embodiments, the step of contacting is performed under conditions that allow IgG from the bodily fluid to bind to at least one component of the food preparation. The method continues with a step of measuring IgG bound to the at least one component of the food preparation to obtain a signal, and then comparing the signal to a gender-stratified reference value for the food preparation using the gender identification to obtain a result. Then, the method also includes a step of updating or generating a report using the result.

Another aspect of the embodiments described herein includes a method of generating a test for food intolerance in patients diagnosed with or suspected to have psoriasis. The method includes a step of obtaining test results for a plurality of distinct food preparations. The test results are based on bodily fluids of patients diagnosed with or suspected to have psoriasis and bodily fluids of a control group not diagnosed with or not suspected to have psoriasis. The method also includes a step of stratifying the test results by gender for each of the distinct food preparations. Then the method continues with a step of assigning for a predetermined percentile rank a different cutoff value for male and female patients for each of the distinct food preparations.

Still another aspect of the embodiments described herein includes a use of a plurality of distinct food preparations coupled to individually addressable respective solid carriers in a diagnosis of psoriasis. The plurality of distinct food preparations are selected based on their average discriminatory p-value of ≤0.07 as determined by raw p-value or an average discriminatory p-value of ≤0.10 as determined by FDR multiplicity adjusted p-value.

Various objects, features, aspects and advantages of the embodiments described herein will become more apparent from the following detailed description of preferred embodiments, along with the accompanying drawing figures in which like numerals represent like components.

### BRIEF DESCRIPTION OF THE DRAWINGS AND TABLES

Table 1 shows a list of food items from which food preparations can be prepared.

Table 2 shows statistical data of foods ranked according to 2-tailed FDR multiplicity-adjusted p-values.

Table 3 shows statistical data of ELISA score by food and gender.

Table 4 shows cutpoint values of foods for a predetermined percentile rank.

FIG. 1A illustrates ELISA signal score of male psoriasis patients and control tested with peach.

FIG. 1B illustrates a distribution of percentage of male psoriasis subjects exceeding the 90^{th} and 95^{th} percentile tested with peach.

FIG. 1C illustrates a signal distribution in women along with the 95^{th} percentile cutoff as determined from the female control population tested with peach.

FIG. 1D illustrates a distribution of percentage of female psoriasis subjects exceeding the 90^{th} and 95^{th} percentile tested with peach.

FIG. 2A illustrates ELISA signal score of male psoriasis patients and control tested with cucumber.

FIG. 2B illustrates a distribution of percentage of male psoriasis subjects exceeding the 90^{th} and 95^{th} percentile tested with cucumber.

FIG. 2C illustrates a signal distribution in women along with the 95^{th} percentile cutoff as determined from the female control population tested with cucumber.

FIG. 2D illustrates a distribution of percentage of female psoriasis subjects exceeding the 90^{th} and 95^{th} percentile tested with cucumber.

FIG. 3A illustrates ELISA signal score of male psoriasis patients and control tested with tea.

FIG. 3B illustrates a distribution of percentage of male psoriasis subjects exceeding the 90^{th} and 95^{th} percentile tested with tea.

FIG. 3C illustrates a signal distribution in women along with the 95^{th} percentile cutoff as determined from the female control population tested with tea.

FIG. 3D illustrates a distribution of percentage of female psoriasis subjects exceeding the 90^{th} and 95^{th} percentile tested with tea.

FIG. 4A illustrates ELISA signal score of male psoriasis patients and control tested with tomato.

FIG. 4B illustrates a distribution of percentage of male psoriasis subjects exceeding the 90^{th} and 95^{th} percentile tested with tomato.

FIG. 4C illustrates a signal distribution in women along with the 95^{th} percentile cutoff as determined from the female control population tested with tomato.

FIG. 4D illustrates a distribution of percentage of female psoriasis subjects exceeding the 90^{th} and 95^{th} percentile tested with tomato.

FIG. 5A illustrates distributions of psoriasis subjects by number of foods that were identified as trigger foods at the 90^{th} percentile.

FIG. 5B illustrates distributions of psoriasis subjects by number of foods that were identified as trigger foods at the 95^{th} percentile.

Table 5A shows raw data of psoriasis patients and control with number of positive results based on the 90^{th} percentile.

Table 5B shows raw data of psoriasis patients and control with number of positive results based on the 95^{th} percentile.

Table 6A shows statistical data summarizing the raw data of psoriasis patient populations shown in Table 5A.

Table 6B shows statistical data summarizing the raw data of psoriasis patient populations shown in Table 5B.

Table 7A shows statistical data summarizing the raw data of control populations shown in Table 5A.

Table 7B shows statistical data summarizing the raw data of control populations shown in Table 5B.

Table 8A shows statistical data summarizing the raw data of psoriasis patient populations shown in Table 5A transformed by logarithmic transformation.

Table 8B shows statistical data summarizing the raw data of psoriasis patient populations shown in Table 5B transformed by logarithmic transformation.

Table 9A shows statistical data summarizing the raw data of control populations shown in Table 5A transformed by logarithmic transformation.

Table 9B shows statistical data summarizing the raw data of control populations shown in Table 5B transformed by logarithmic transformation.

Table 10A shows statistical data of an independent T-test to compare the geometric mean number of positive foods between the psoriasis and non-psoriasis samples based on the 90^{th} percentile.

Table 10B shows statistical data of an independent T-test to compare the geometric mean number of positive foods between the psoriasis and non-psoriasis samples based on the 95^{th} percentile.

Table 11A shows statistical data of a Mann-Whitney test to compare the geometric mean number of positive foods between the psoriasis and non-psoriasis samples based on the 90^{th} percentile.

Table 11B shows statistical data of a Mann-Whitney test to compare the geometric mean number of positive foods between the psoriasis and non-psoriasis samples based on the 95^{th} percentile.

FIG. 6A illustrates a box and whisker plot of data shown in Table 5A.

FIG. 6B illustrates a notched box and whisker plot of data shown in Table 5A.

FIG. 6C illustrates a box and whisker plot of data shown in Table 5B.

FIG. 6D illustrates a notched box and whisker plot of data shown in Table 5B.

Table 12A shows statistical data of a Receiver Operating Characteristic (ROC) curve analysis of data shown in Tables 5A-11A.

Table 12B shows statistical data of a Receiver Operating Characteristic (ROC) curve analysis of data shown in Tables 5B-11B.

FIG. 7A illustrates the ROC curve corresponding to the statistical data shown in Table 12A.

FIG. 7B illustrates the ROC curve corresponding to the statistical data shown in Table 12B.

Table 13A shows a statistical data of performance metrics in predicting psoriasis status among female patients from number of positive foods based on the 90^{th} percentile.

Table 13B shows a statistical data of performance metrics in predicting psoriasis status among male patients from number of positive foods based on the 90^{th} percentile.

Table 14A shows a statistical data of performance metrics in predicting psoriasis status among female patients from number of positive foods based on the 95^{th} percentile.

Table 14B shows a statistical data of performance metrics in predicting psoriasis status among male patients from number of positive foods based on the 95^{th} percentile

### DETAILED DESCRIPTION

The inventors have discovered that food preparations used in certain food tests to identify trigger foods in patients diagnosed with or suspected to have psoriasis are not necessarily predictive of, or otherwise associated with, psoriasis symptoms. Indeed, various experiments have revealed that among a wide variety of food items, certain food items are highly predictive/associated with psoriasis, whereas others may have no statistically significant association with psoriasis.

Even more unexpectedly, the inventors discovered that in addition to the high variability of food items, gender variability with respect to response in a test may play a substantial role in the determination of association of a food item with psoriasis. Consequently, based on the inventors' findings and further contemplations, test kits and methods are now presented with substantially higher predictive power in the choice of food items that could be eliminated for reduction of psoriasis signs and symptoms.

Food sensitivity (also known as food intolerance), especially as it relates to psoriasis (a type of autoimmune disease), often presents with skin lesions, scaly patches, papules, and plaques that usually itch. The underlying causes of psoriasis are not well understood in the medical community. Psoriasis may be visually diagnosed, along with various tests to exclude various other inflammatory or infectious conditions. Unfortunately, treatment of psoriasis may often be less than effective and may present new difficulties due to immune suppressive or modulatory effects. In certain instances, elimination of other one or more food items has also shown promise in at least reducing incidence and/or severity of the symptoms. However, psoriasis is often quite diverse with respect to dietary items triggering symptoms, whereby no standardized test to help identify trigger food items with a reasonable degree of certainty is known, thus leaving such patients often to trial-and-error.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities or ranges, used to describe and claim certain embodiments of the disclosure are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the disclosure are approximations, the numerical values set forth in the specific examples are reported as precisely as practicable. The numerical values presented in some embodiments of the disclosure may contain certain errors necessarily resulting from the standard deviation found in their respective testing measurements. Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In one aspect, the inventors therefore contemplate a test kit or test panel that is suitable for testing food intolerance in a patient that is diagnosed with or suspected to have psoriasis. Such a test kit or panel will include one or more distinct food preparations (e.g., raw or processed extract, which may include an aqueous extract with optional co-solvent, which may or may not be filtered) that are coupled to (e.g., immobilized on) individually addressable respective solid carriers (e.g., in a form of an array or a micro well plate), wherein each distinct food preparation has an average discriminatory p-value of ≤0.07 as determined by raw p-value or an average discriminatory p-value of ≤0.10 as determined by FDR multiplicity adjusted p-value. In certain embodiments, the average discriminatory p-value is determined by comparing assay values of a first patient test cohort that is diagnosed with or suspected of having psoriasis, with assay values of a second patient test cohort that is not diagnosed with or suspected of having psoriasis. In such embodiments, the assay values can be determined by conducting assays for the first and second patient test cohorts with the distinct food preparation.

In some embodiments, the numbers expressing quantities of ingredients, properties such as concentration, reaction conditions, and so forth, used to describe and claim certain embodiments of the disclosure are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that, the numerical ranges and parameters setting forth the broad scope of some embodiments of the disclosure are approximations, the numerical values set forth in the specific examples are reported as precisely as practicable. The numerical values presented in some embodiments of the disclosure may contain certain errors necessarily resulting from the standard deviation found in their respective testing measurements. Moreover, and unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

While not limiting to the inventive subject matter, food preparations will typically be drawn from foods generally known or suspected to trigger signs or symptoms of psoriasis. Particularly suitable food preparations may be identified by the experimental procedures outlined below. Thus, it should be appreciated that the food items need not be limited to the items described herein, but that all items are contemplated that can be identified by the methods presented herein. Therefore, exemplary food preparations include at least two, at least four, at least eight, or at least 12 food preparations prepared from foods 1-59 of Table 2. Thus, for example, in some embodiments, the exemplary food preparations can include at least two of peach, cucumber, tea, tomato, broccoli, cauliflower, almond, green pepper, grapefruit, tobacco, eggplant, rye, oat, cantaloupe, cabbage, cane sugar, sweet pot, pineapple, avocado, orange, spinach, honey, swiss cheese, malt, mustard, wheat, apple, chocolate, yogurt, and goat milk. Still further especially contemplated food items and food additives from which food preparations can be prepared are listed in Table 1.

Using bodily fluids from patients diagnosed with or suspected of having psoriasis, and a healthy control group individuals (i.e., those not diagnosed with or not suspected to have psoriasis), numerous additional food items may be identified. In certain embodiments, the methods described herein comprise the one of one or more distinct food preparations having an average discriminatory p-value, wherein the average discriminatory p-value for each distinct food preparation is determined by a process that includes comparing test results of a first patient test cohort that is diagnosed with or suspected of having psoriasis, with test results of a second patient test cohort that is not diagnosed with or suspected of having psoriasis. In such embodiments, test results (e.g., ELISA) for the first and second patient test cohorts are obtained for various distinct food preparations, wherein the test results are based on contacting bodily fluids (e.g., blood saliva, fecal suspension) of the first patient test cohort and the second patient test cohort with each food preparation.

In certain embodiments, such identified food preparations will have high discriminatory power and, as such, will have a p-value of ≤0.15, ≤0.10, or even ≤0.05 as determined by raw p-value, and/or a p-value of ≤0.10, ≤0.08, or even ≤0.07 as determined by False Discovery Rate (FDR) multiplicity adjusted p-value.

Therefore, where a panel has multiple food preparations, it is contemplated that each distinct food preparations will have an average discriminatory p-value of ≤0.05 as determined by raw p-value or an average discriminatory p-value of ≤0.08 as determined by FDR multiplicity adjusted p-value, or even an average discriminatory p-value of ≤0.025 as determined by raw p-value or an average discriminatory p-value of ≤0.07 as determined by FDR multiplicity adjusted p-value. In certain aspects, it should be appreciated that the FDR multiplicity adjusted p-value may be adjusted for at least one of age or gender, and in certain embodiments adjusted for both age and gender. On the other hand, where a test kit or panel is stratified for use with a single gender, it is also contemplated that in a test kit or panel at least 50% (or 70% or all) of the plurality of distinct food preparations, when adjusted for a single gender, have an average discriminatory p-value of ≤0.07 as determined by raw p-value or an average discriminatory p-value of ≤0.10 as determined by FDR multiplicity adjusted p-value. Furthermore, it should be appreciated that other stratifications (e.g., dietary preference, ethnicity, place of residence, genetic predisposition or family history, etc.) are also contemplated, and a person of ordinary skill in the art will be readily apprised of the appropriate choice of stratification.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the disclosure and does not pose a limitation on the scope of the disclosure otherwise claimed. No language in the specification should be construed as indicating any non-claimed element essential to the practice of the disclosure.

Of course, it should be noted that the particular format of the test kit or panel may vary considerably, and contemplated formats include micro well plates, dip sticks, membrane-bound arrays, etc. Consequently, the solid carrier to which the food preparations are coupled may include wells of a multiwall plate, a bead (e.g., color-coded or magnetic), an adsorptive film (e.g., nitrocellulose or micro/nanoporous polymeric film), or an electrical sensor (e.g. a printed copper sensor or microchip).

Consequently, the inventors also contemplate a method of testing food intolerance in patients that are diagnosed with or suspected to have psoriasis. Most typically, such methods will include a step of contacting a food preparation with a bodily fluid (e.g., whole blood, plasma, serum, saliva, or a fecal suspension) of a patient that is diagnosed with or suspected to have psoriasis, and wherein the bodily fluid is associated with a gender identification. As noted before, the step of contacting can be performed under conditions that allow an immunoglobulin such as IgG (or IgE or IgA or IgM) from the bodily fluid to bind to at least one component of the food preparation, and the IgG bound to the component(s) of the food preparation are then quantified/measured to obtain a signal. In some embodiments, the signal is then compared against a gender-stratified reference value (e.g., at least a 90th percentile value) for the food preparation using the gender identification to obtain a result, which is then used to update or generate a report (e.g., written medical report; oral report of results from doctor to patient; written or oral directive from physician based on results).

In certain embodiments, such methods will not be limited to a single food preparation, but will employ multiple different food preparations. As noted before, suitable food preparations can be identified using various methods as described below; however, certain food preparations may include foods 1-59 of Table 2, and/or items of Table 1. As also noted above, in certain embodiments at least some, or all of the different food preparations have an average discriminatory p-value of ≤0.07 (or ≤0.05, or ≤0.025) as determined by raw p-value, and/or or an average discriminatory p-value of ≤0.10 (or ≤0.08, or ≤0.07) as determined by FDR multiplicity adjusted p-value.

While in certain embodiments food preparations are prepared from single food items as crude extracts, or crude filtered extracts, it is contemplated that food preparations can be prepared from mixtures of a plurality of food items (e.g., a mixture of citrus comprising lemon, orange, and a grapefruit, a mixture of yeast comprising baker's yeast and brewer's yeast, a mixture of rice comprising a brown rice and white rice, a mixture of sugars comprising honey, malt, and cane sugar. In some embodiments, it is also contemplated that food preparations can be prepared from purified food antigens or recombinant food antigens.

the each food preparation is immobilized on a solid surface (typically in an addressable manner, such that each food preparation is isolated), it is contemplated that the step of measuring the IgG or other type of antibody bound to the component of the food preparation is performed via an ELISA (enzyme-linked immunosorbent assay) test. Exemplary solid surfaces include, but are not limited to, wells in a multiwell plate, such that each food preparation may be isolated to a separate microwell. In certain embodiments, the food preparation will be coupled to, or immobilized on, the solid surface. In other embodiments, the food preparation(s) will be coupled to a molecular tag that allows for binding to human immunoglobulins (e.g., IgG, etc.) in solution.

Viewed from a different perspective, the inventors also contemplate a method of generating a test for food intolerance in patients diagnosed with or suspected to have psoriasis. Such a test is applied to patients already diagnosed with or suspected to have psoriasis, in certain embodiments, the authors do not contemplate that the method has a diagnostic purpose. Instead, the method is for identifying triggering food items among already diagnosed or suspected psoriasis patients. As with the other methods described herein, test kits that can be used for this method may comprise one or more distinct food preparations having an average discriminatory p-value, wherein the average discriminatory p-value for each distinct food preparation is determined by a process that includes comparing test results of a first patient test cohort that is diagnosed with or suspected of having psoriasis, with test results of a second patient test cohort that is not diagnosed with or suspected of having psoriasis. In such embodiments, test results (e.g., ELISA, etc.) for the first and second patient test cohorts are obtained for various distinct food preparations, wherein the test results are based on contacting bodily fluids (e.g., blood saliva, fecal suspension, etc.) of the first patient test cohort and the second patient test cohort with each food preparation. In certain embodiments, the test results are then stratified by gender for each of the distinct food preparations, a different cutoff value for male and female patients for each of the distinct food preparations (e.g., cutoff value for male and female patients has a difference of at least 10% (abs), etc.) is assigned for a predetermined percentile rank (e.g., 90th or 95th percentile, etc.).

As noted earlier, in certain embodiments, it is contemplated that the distinct food preparations include at least two (or six, or ten, or fifteen) food preparations prepared from food items selected from the group consisting of foods 1-59 of Table 2, and/or items of Table 1. On the other hand, where new food items are tested, it should be appreciated that the distinct food preparations include a food preparation prepared from a food items other than foods 1-59 of Table 2. Regardless of the particular choice of food items, in certain embodiments each distinct food preparation will have an average discriminatory p-value of ≤0.07 (or ≤0.05, or ≤0.025) as determined by raw p-value or an average discriminatory p-value of ≤0.10 (or ≤0.08, or ≤0.07) as determined by FDR multiplicity adjusted p-value. Exemplary aspects and protocols, and considerations are provided in the experimental description below.

Thus, it should be appreciated that by having a high-confidence test system as described herein, the rate of false-positive and false negatives can be significantly reduced, and especially where the test systems and methods are gender stratified or adjusted for gender differences as shown below. Such advantages have heretofore not been realized and it is expected that the systems and methods presented herein will substantially increase the predictive power of food sensitivity tests for patients diagnosed with or suspected to have psoriasis.

### Experiments

General Protocol for Food Preparation Generation:

Commercially available food extracts (available from Biomerica Inc., 17571 Von Karman Ave, Irvine, Calif. 92614) prepared from the edible portion of the respective raw foods were used to prepare ELISA plates following the manufacturer's instructions.

For some food extracts, the inventors expect that food extracts prepared with specific procedures to generate food extracts may provides more superior results in detecting elevated IgG reactivity in psoriasis patients compared to commercially available food extracts. For example, for grains and nuts, a three-step procedure of generating food extracts may provide more accurate results. The first step is a defatting step. In this step, lipids from grains and nuts are extracted by contacting the flour of grains and nuts with a non-polar solvent and collecting residue. Then, the defatted grain or nut flour are extracted by contacting the flour with elevated pH to obtain a mixture and removing the solid from the mixture to obtain the liquid extract. Once the liquid extract is generated, the liquid extract is stabilized by adding an aqueous formulation. In one embodiment, the aqueous formulation includes a sugar alcohol, a metal chelating agent, protease inhibitor, mineral salt, and buffer component 20-50 mM of buffer from 4-9 pH. This formulation allowed for long term storage at −70° C. and multiple freeze-thaws without a loss of activity.

For another example, for meats and fish, a two-step procedure of generating food extract may provide more accurate results. The first step is an extraction step. In this step, extracts from raw, uncooked meats or fish are generated by emulsifying the raw, uncooked meats or fish in an aqueous buffer formulation in a high impact pressure processor. Then, solid materials are removed to obtain liquid extract. Once the liquid extract is generated, the liquid extract is stabilized by adding an aqueous formulation. In one embodiment, the aqueous formulation includes a sugar alcohol, a metal chelating agent, protease inhibitor, mineral salt, and buffer component 20-50 mM of buffer from 4-9 pH. This formulation allowed for long term storage at −70° C. and multiple freeze-thaws without a loss of activity.

For still another example, for fruits and vegetables, a two-step procedure of generating food extract is may provide more accurate results. The first step is an extraction step. In this step, liquid extracts from fruits or vegetables are generated using an extractor (e.g., masticating juicer, etc) to pulverize foods and extract juice. Then, solid materials are removed to obtain liquid extract. Once the liquid extract is generated, the liquid extract is stabilized by adding an aqueous formulation. In one embodiment, the aqueous formulation includes a sugar alcohol, a metal chelating agent, protease inhibitor, mineral salt, and buffer component 20-50 mM of buffer from 4-9 pH. This formulation allowed for long term storage at −70° C. and multiple freeze-thaws without a loss of activity.

Blocking of ELISA Plates:

To optimize signal to noise, plates will be blocked with a proprietary blocking buffer. In one embodiment, the blocking buffer includes 20-50 mM of buffer from 4-9 pH, a protein of animal origin (e.g., beef, chicken) and a short chain alcohol (e.g., glycerin, etc.). Other blocking buffers, including several commercial preparations, can be attempted but may not provide adequate signal to noise and low assay variability required.

ELISA Preparation and Sample Testing:

Food antigen preparations were immobilized onto respective microtiter wells following the manufacturer's instructions. For the assays (e.g., multiplexed assays, etc), the food antigens were allowed to react with antibodies present in the patients' serum, and excess serum proteins were removed by a wash step. For detection of IgG antibody binding, enzyme labeled anti-IgG antibody conjugate was allowed to react with antigen-antibody complex. A color was developed by the addition of a substrate that reacts with the coupled enzyme. The color intensity was measured and is directly proportional to the concentration of IgG antibody specific to a particular food antigen.

Methodology to Determine Ranked Food List in Order of Ability of ELISA Signals to Distinguish Psoriasis from Control Subjects:

Out of an initial selection (e.g., 100 food items, or 150 food items, or even more), samples can be eliminated prior to analysis due to low consumption in an intended population. In addition, specific food items can be used as being representative of a larger more generic food group, especially where prior testing has established a correlation among different species within a generic group (with respect to both genders, or correlation with a single gender). For example, Thailand Shrimp could be dropped in favor of U.S. Gulf White Shrimp as representative of the “shrimp” food group, or King Crab could be dropped in favor of Dungeness Crab as representative of the “crab” food group. In further aspects, the final list foods will be shorter than 50 food items, or equal or less than of 40 food items.

For each of the tested foods, signal scores will be compared between psoriasis and controls using a permutation test on a two-sample t-test with a relative high number of resamplings (e.g., >1,000, or >10,000, or even >50,000). The Satterthwaite approximation can then be used for the denominator degrees of freedom to account for lack of homogeneity of variances, and the 2-tailed permuted p-value will represent the raw p-value for each food. False Discovery Rates (FDR) among the comparisons, will be adjusted by any acceptable statistical procedures (e.g., Benjamini-Hochberg, Family-wise Error Rate (FWER), Per Comparison Error Rate (PCER), etc.).

Foods were then ranked according to their 2-tailed FDR multiplicity-adjusted p-values. Foods with adjusted p-values equal to or lower than the desired FDR threshold are deemed to have significantly higher signal scores among psoriasis than control subjects and therefore deemed candidates for inclusion into a food intolerance panel. A typical result that is representative of the outcome of the statistical procedure is provided in Table 2. Here, the ranking of foods is according to 2-tailed permutation T-test p-values with FDR adjustment.

Based on earlier experiments (data not shown here; see U.S. 62/079,783, which is incorporated herein by reference in its entirety for all purposes), the inventors contemplate that even for the same food preparation tested, the ELISA score for at least several food items will vary dramatically, and exemplary raw data are provided in Table 3. As should be readily appreciated, data unstratified by gender will therefore lose significant explanatory power where the same cutoff value is applied to raw data for male and female data. To overcome such disadvantage, the inventors therefore contemplate stratification of the data by gender as described below.

Statistical Method for Cutpoint Selection for Each Food:

The determination of what ELISA signal scores would constitute a “positive” response can be made by summarizing the distribution of signal scores among the Control subjects. For each food, psoriasis subjects who have observed scores greater than or equal to selected quantiles of the Control subject distribution will be deemed “positive”. To attenuate the influence of any one subject on cutpoint determination, each food-specific and gender-specific dataset will be bootstrap resampled 1,000 times. Within each bootstrap replicate, the 90th and 95th percentiles of the Control signal scores will be determined. Each psoriasis subject in the bootstrap sample will be compared to the 90th and 95% percentiles to determine whether he/she had a “positive” response. The final 90th and 95th percentile-based cutpoints for each food and gender will be computed as the average 90th and 95th percentiles across the 1000 samples. The number of foods for which each psoriasis subject will be rated as “positive” was computed by pooling data across foods. Using such method, the inventors will be now able to identify cutoff values for a predetermined percentile rank that in most cases was substantially different as can be taken from Table 4.

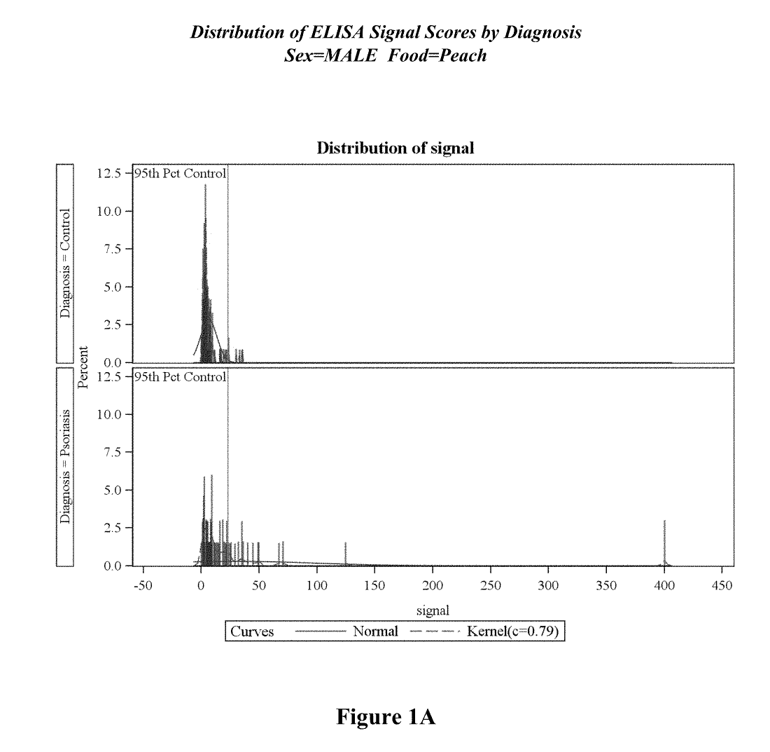

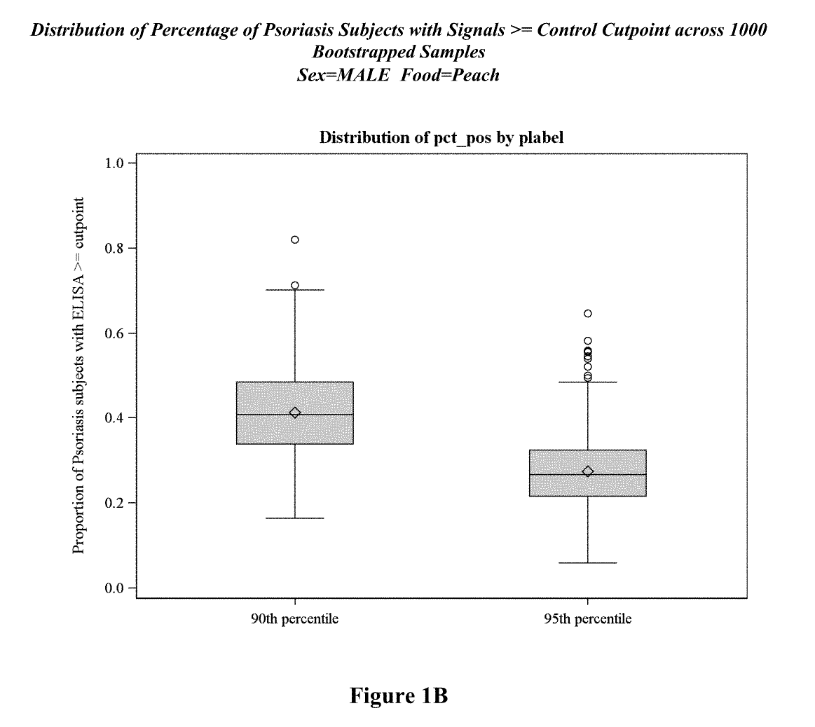

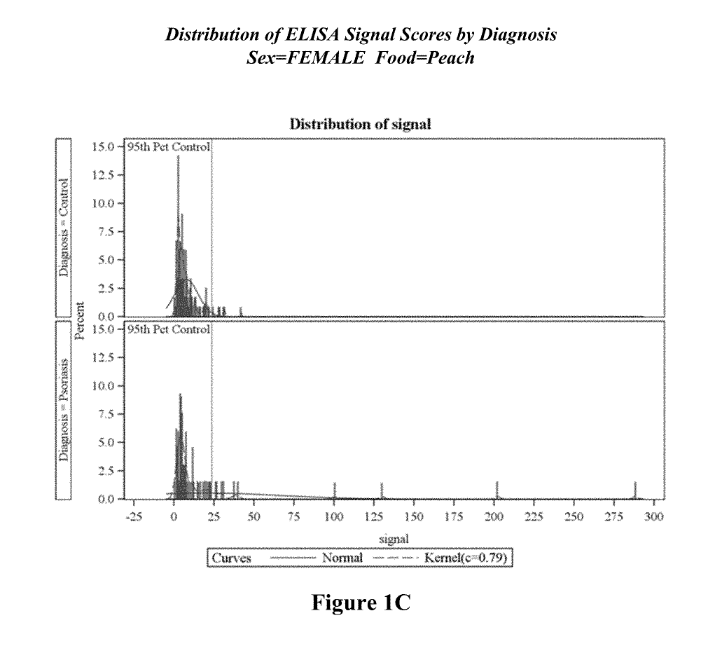

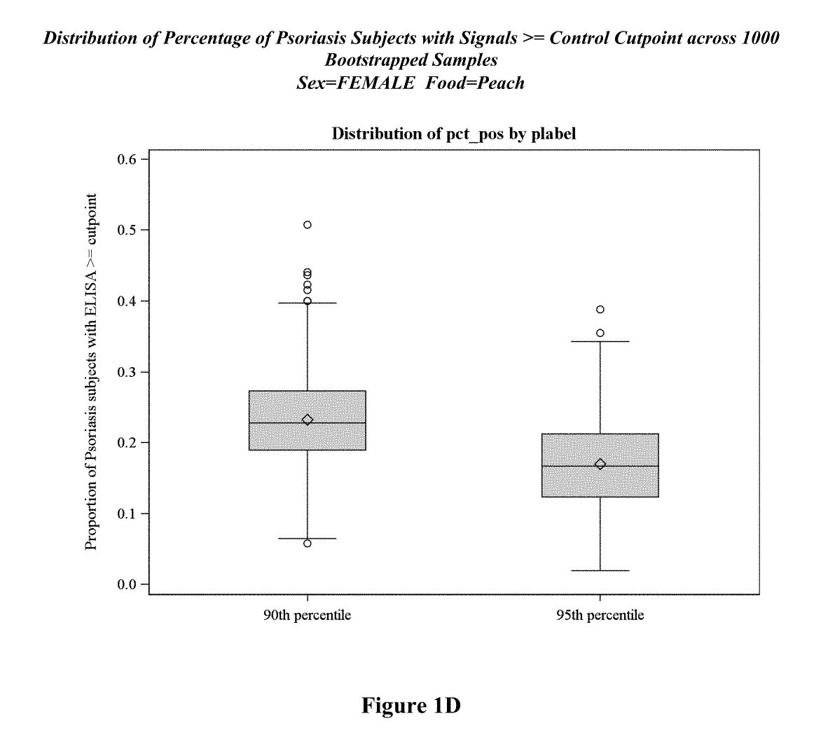

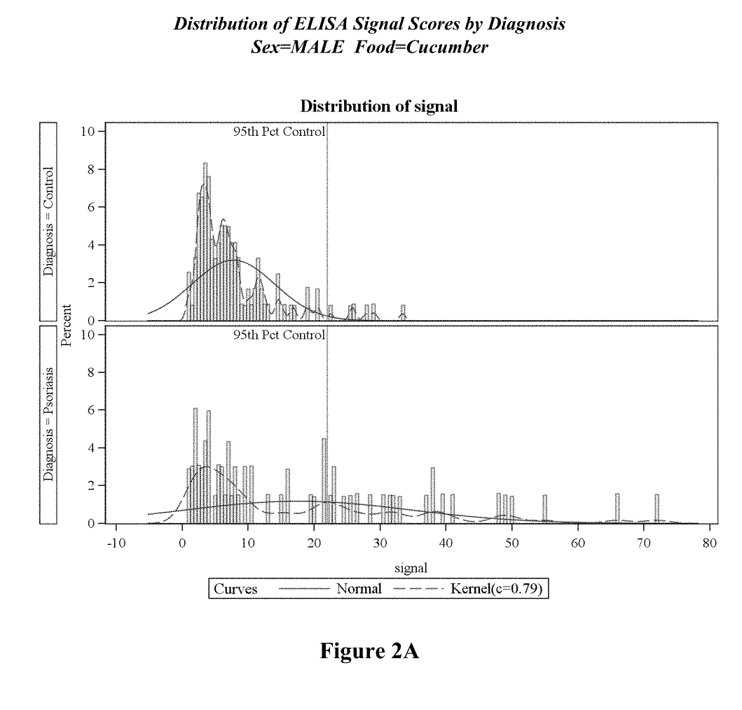

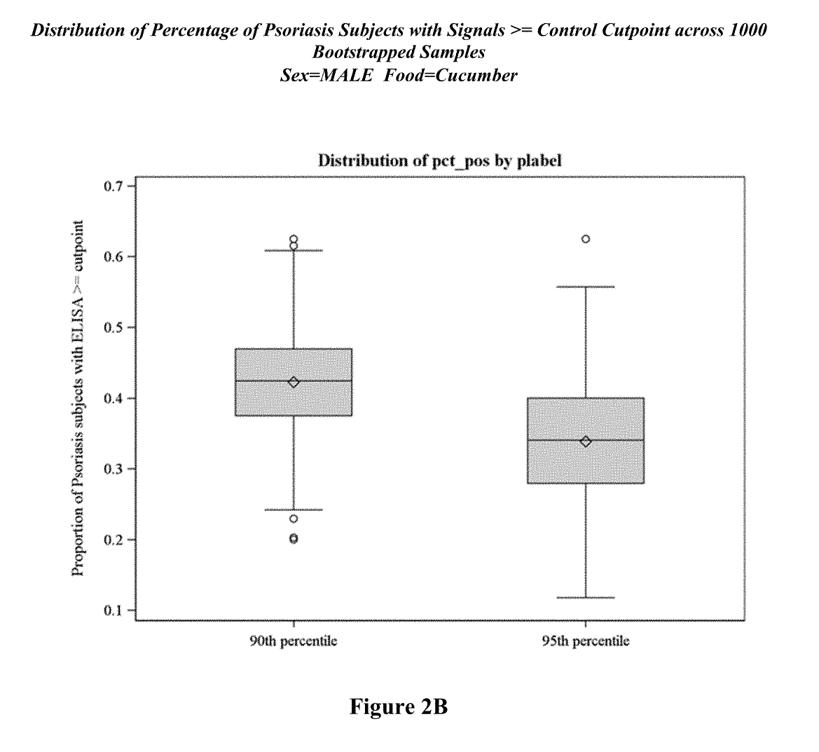

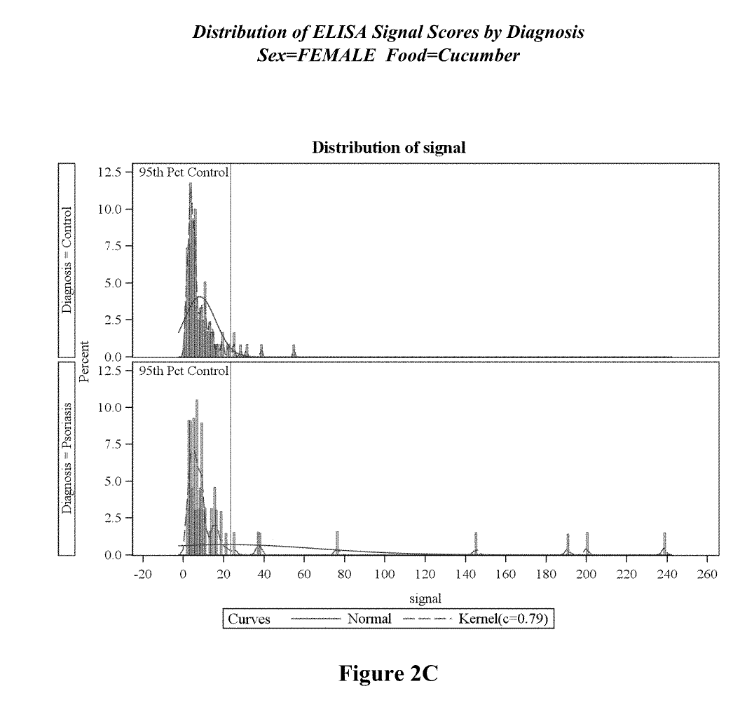

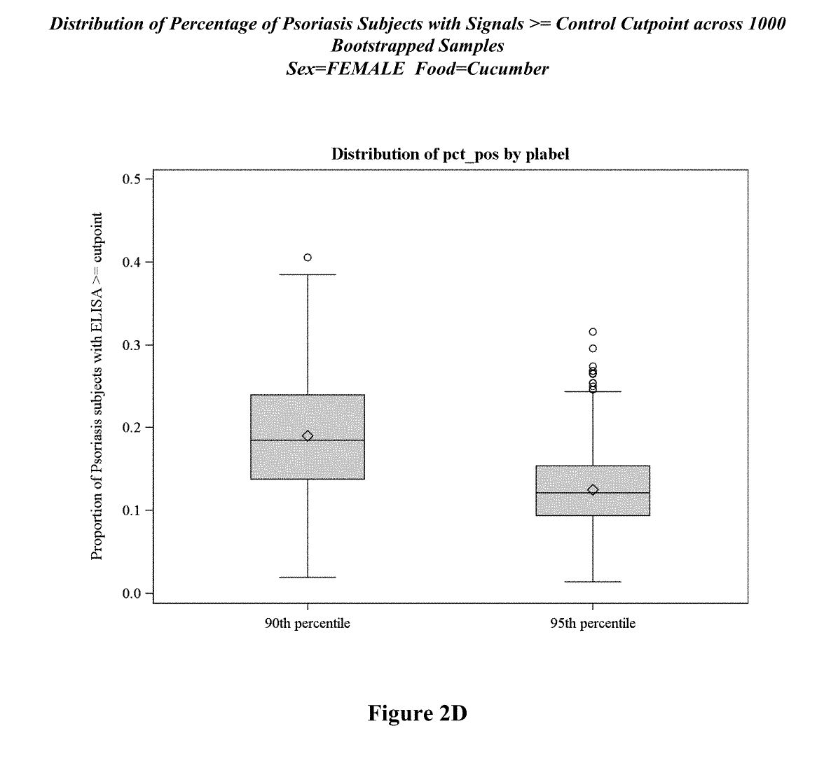

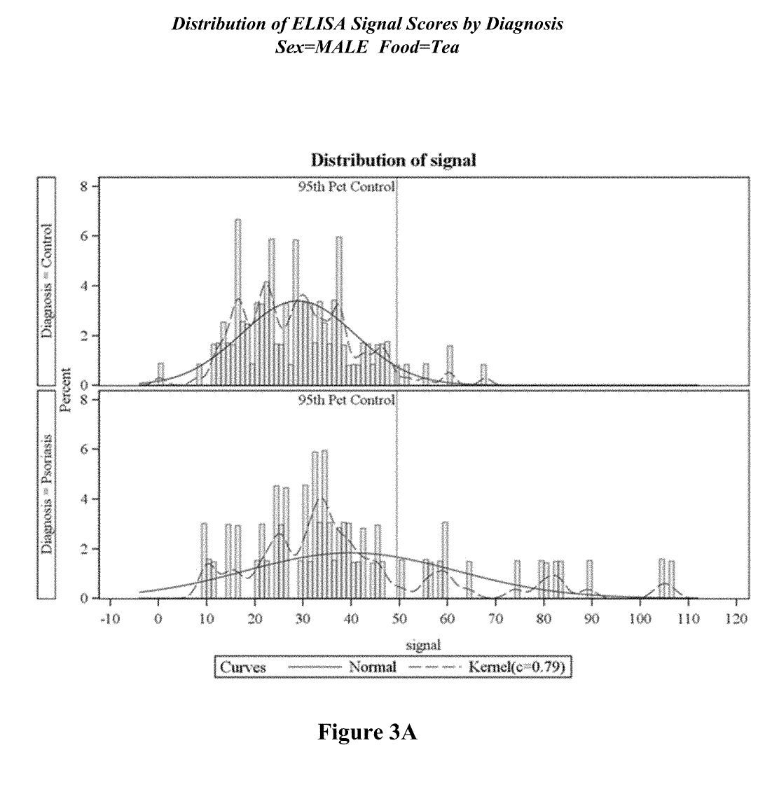

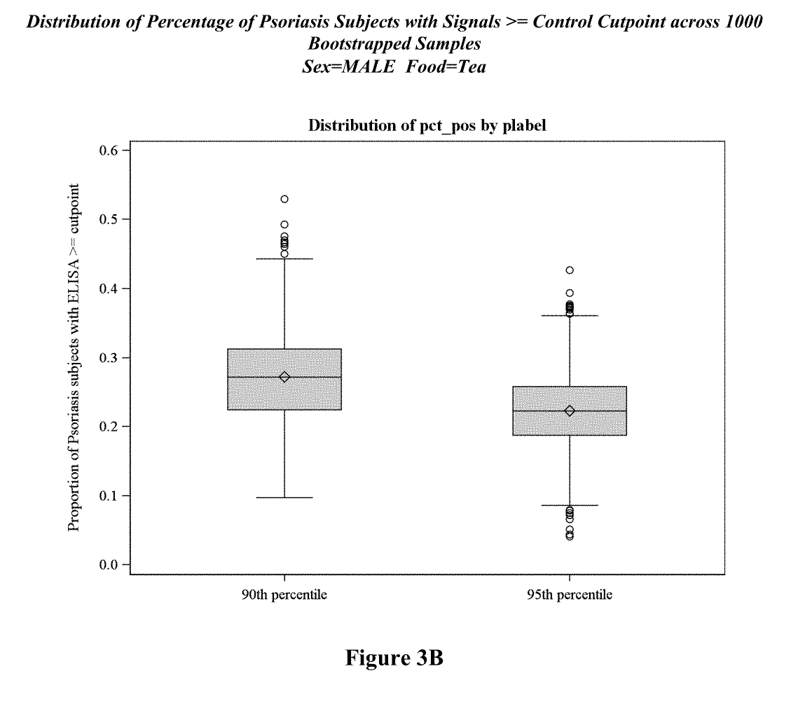

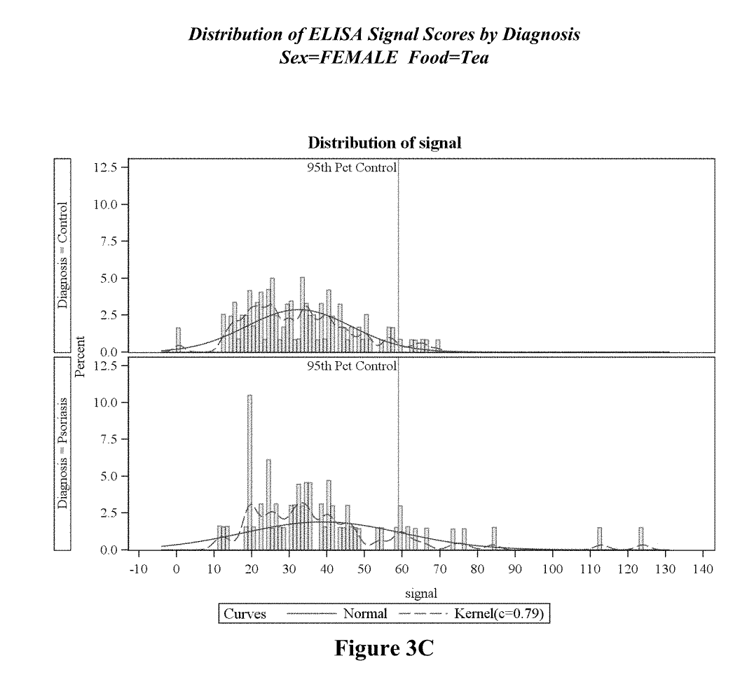

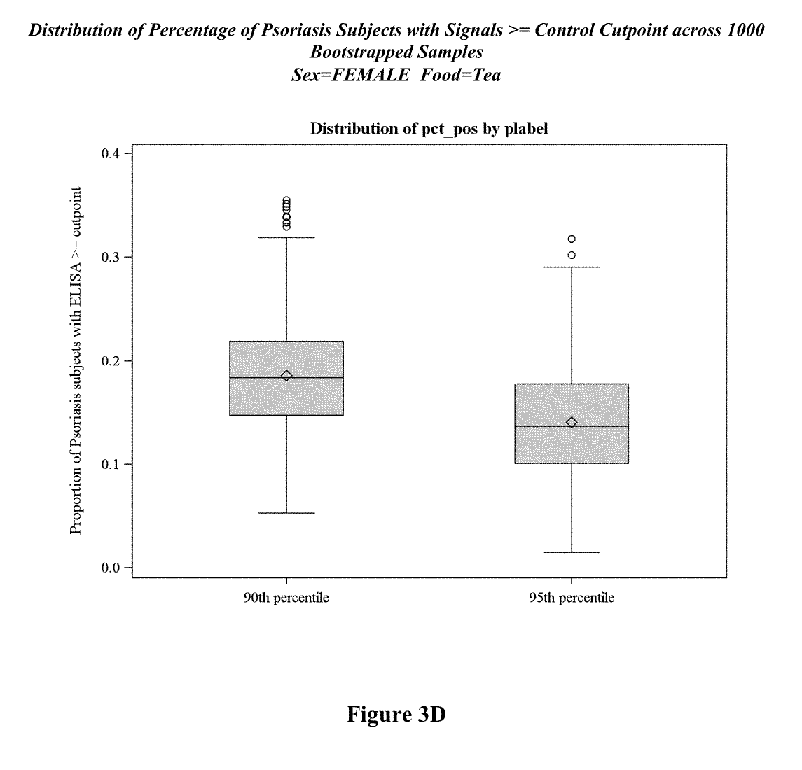

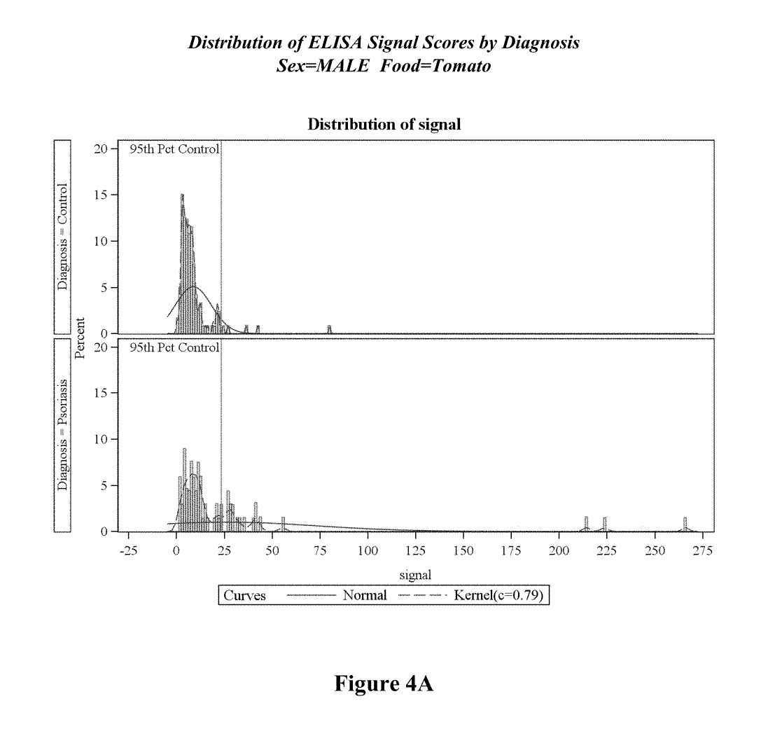

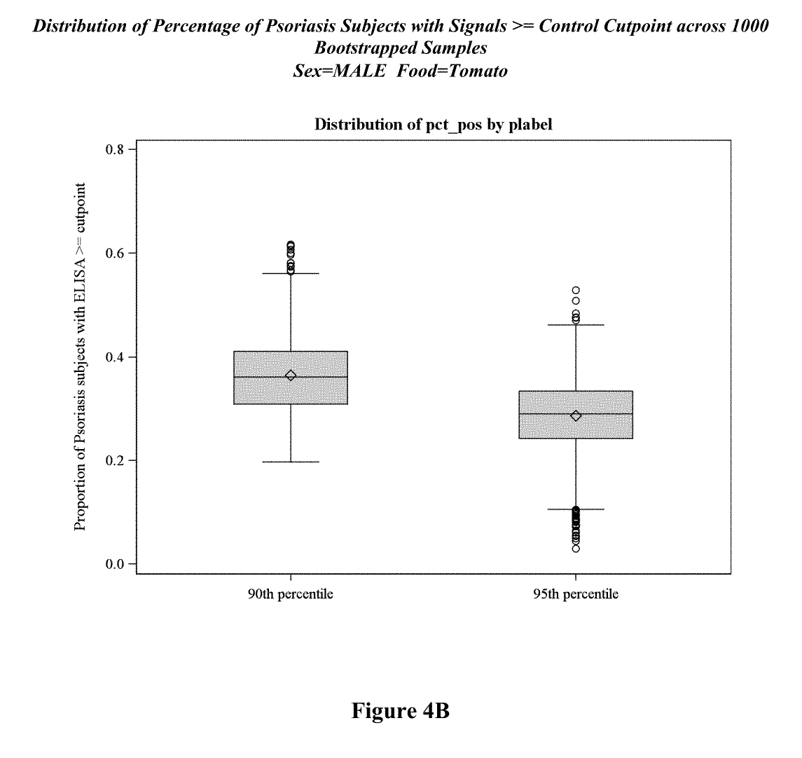

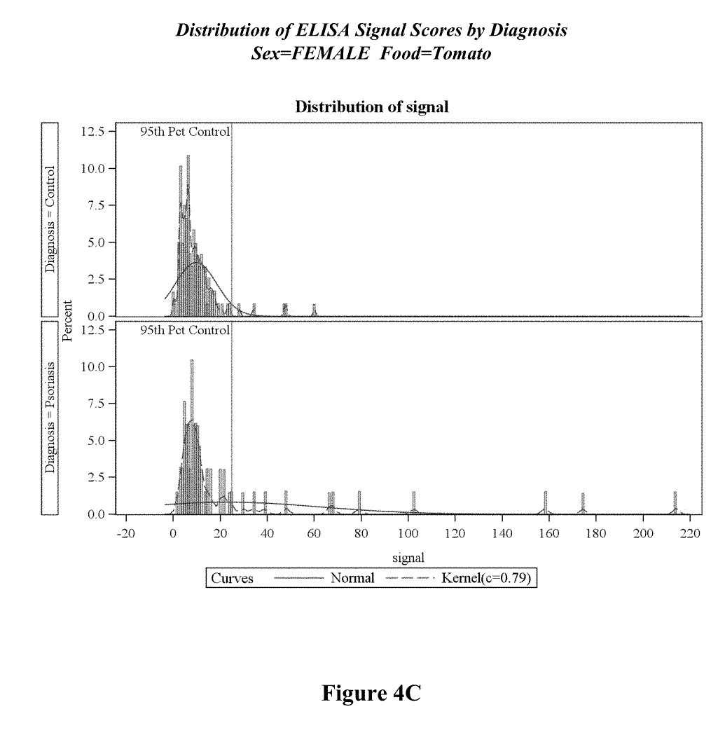

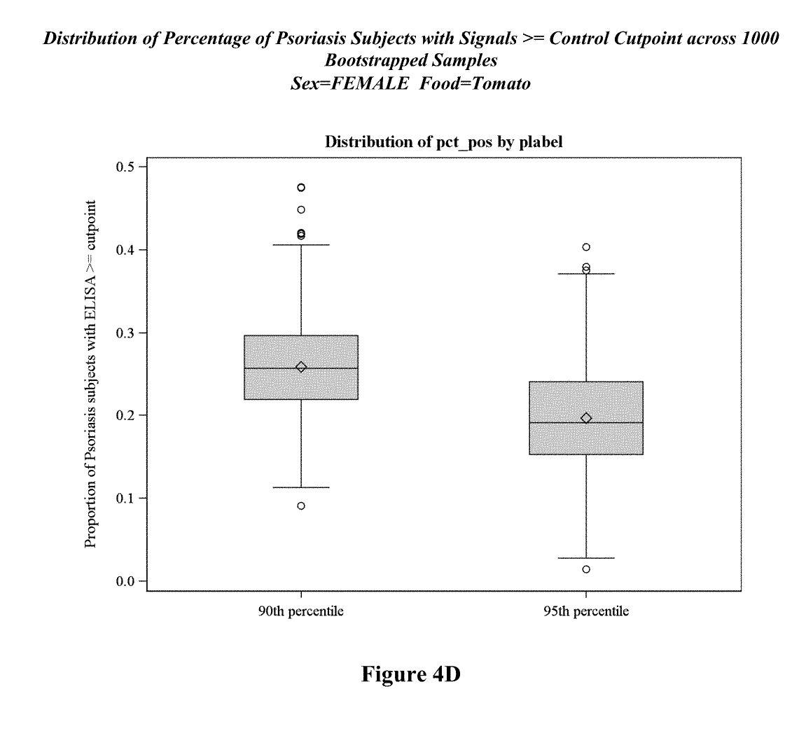

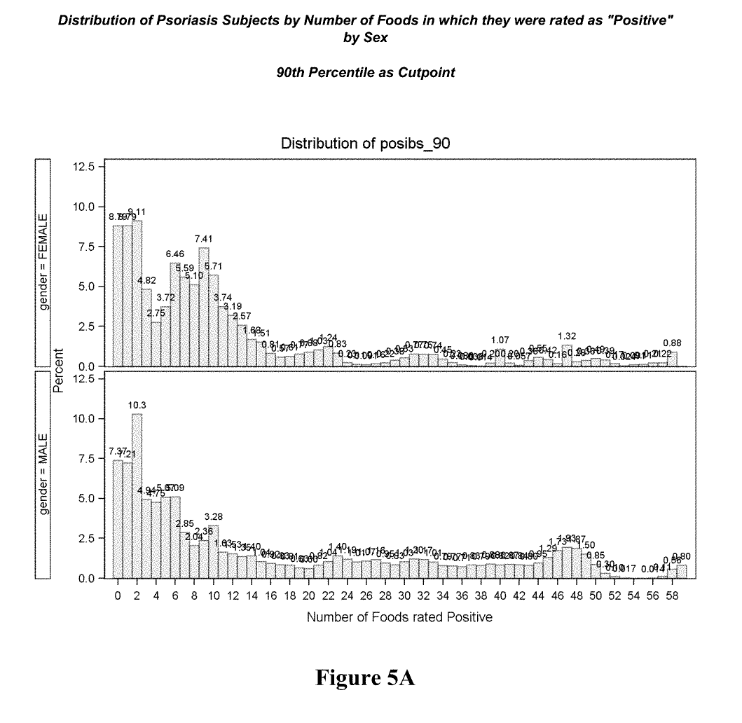

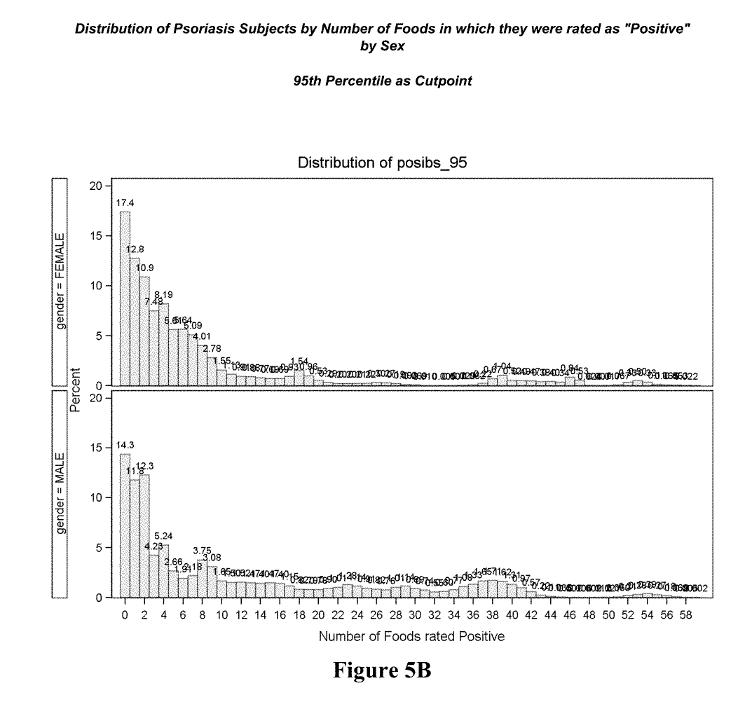

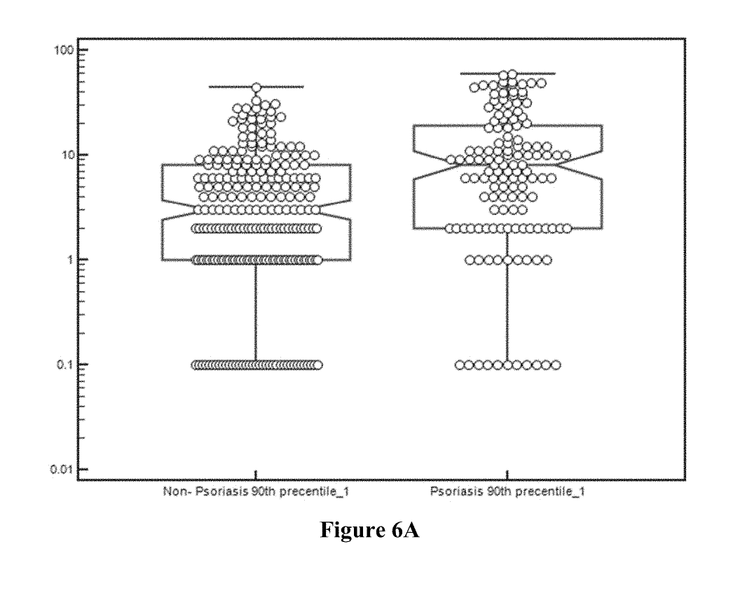

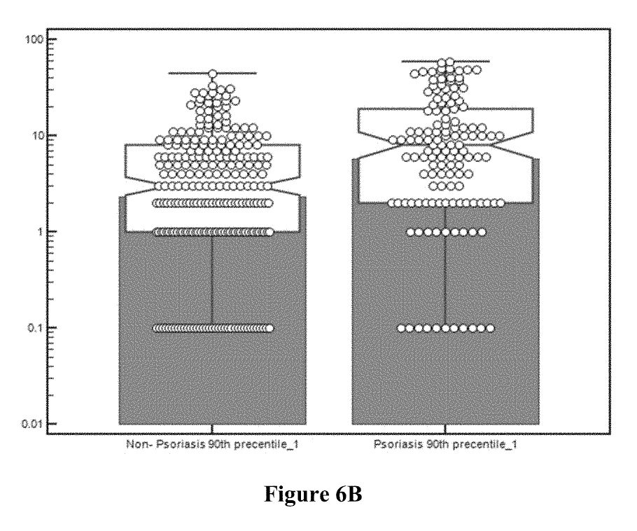

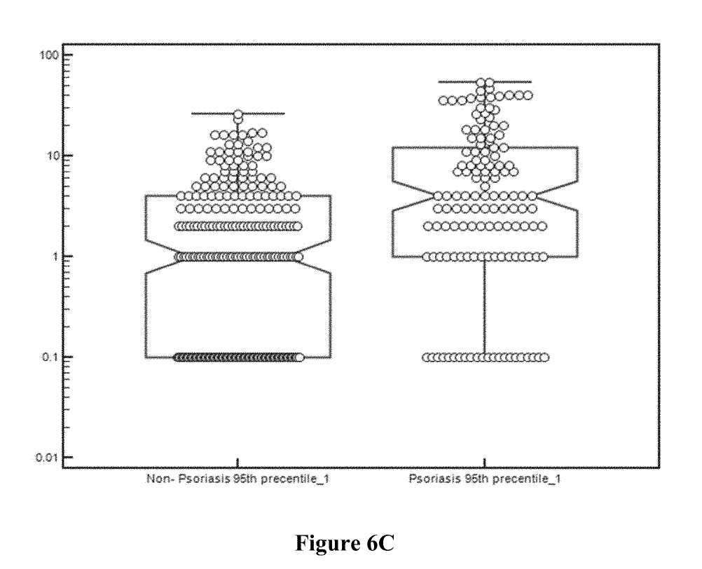

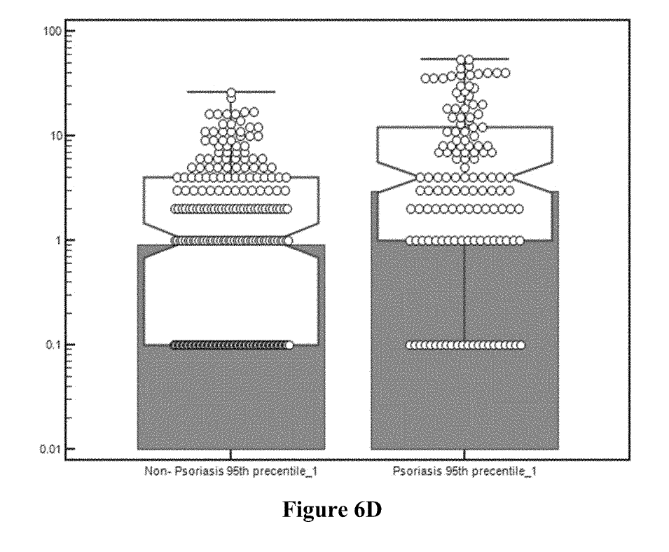

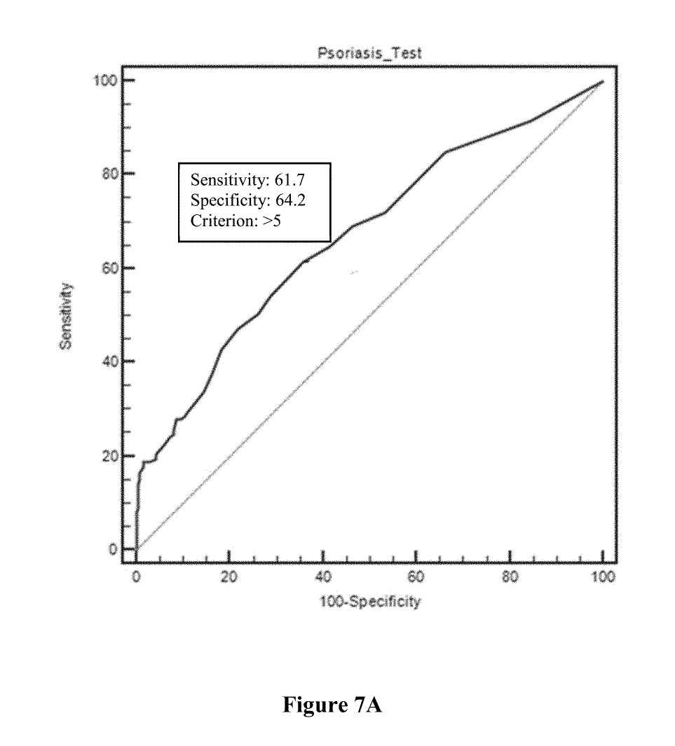

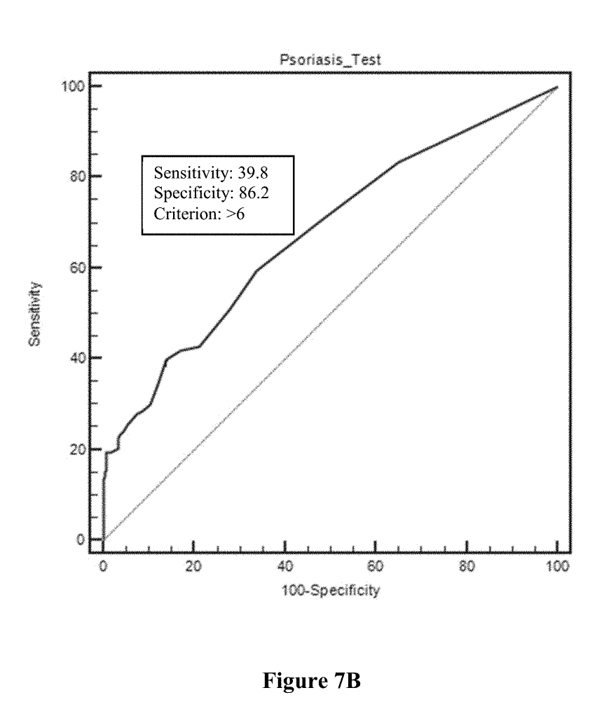

Typical examples for the gender difference in IgG response in blood with respect to peach is shown in FIGS. 1A-1D, where FIG. 1A shows the signal distribution in men along with the 95^{th} percentile cutoff as determined from the male control population. FIG. 1B shows the distribution of percentage of male psoriasis subjects exceeding the 90^{th} and 95^{th} percentile, while FIG. 1C shows the signal distribution in women along with the 95^{th} percentile cutoff as determined from the female control population. FIG. 1D shows the distribution of percentage of female psoriasis subjects exceeding the 90^{th} and 95^{th} percentile. In the same fashion, FIGS. 2A-2D exemplarily depict the differential response to cucumber, FIGS. 3A-3D exemplarily depict the differential response to tea, and FIGS. 4A-4D exemplarily depict the differential response to tomato. FIGS. 5A-5B show the distribution of psoriasis subjects by number of foods that were identified as trigger foods at the 90^{th} percentile (5A) and 95^{th} percentile (5B). Inventors contemplate that regardless of the particular food items, male and female responses were notably distinct.

It should be noted that nothing in the art has provided any predictable food groups related to psoriasis that are gender-stratified. Thus, a discovery of food items that show distinct responses by gender is a surprising result, which was not expected by the inventors. In other words, selection of food items based on gender stratification provides an unexpected technical effect such that statistical significances for particular food items as triggering foods among male or female psoriasis patients have been significantly improved.

Normalization of IgG Response Data:

While the raw data of the patient's IgG response results can be use to compare strength of response among given foods, it is also contemplated that the IgG response results of a patient are normalized and indexed to generate unit-less numbers for comparison of relative strength of response to a given food. For example, one or more of a patient's food specific IgG results (e.g., IgG specific to tomato and IgG specific to cucumber) can be normalized to the patient's total IgG. The normalized value of the patient's IgG specific to tomato can be 0.1 and the normalized value of the patient's IgG specific to cucumber can be 0.3. In this scenario, the relative strength of the patient's response to cucumber is three times higher compared to tomato. Then, the patient's sensitivity to cucumber and tomato can be indexed as such.

In other examples, one or more of a patient's food specific IgG results (e.g., IgG specific to shrimp and IgG specific to pork) can be normalized to the global mean of that patient's food specific IgG results. The global means of the patient's food specific IgG can be measured by total amount of the patient's food specific IgG. In this scenario, the patient's specific IgG to shrimp can be normalized to the mean of patient's total food specific IgG (e.g., mean of IgG levels to shrimp, pork, Dungeness crab, chicken, peas, etc.). However, it is also contemplated that the global means of the patient's food specific IgG can be measured by the patient's IgG levels to a specific type of food via multiple tests. If the patient has been tested for his sensitivity to shrimp five times and to pork seven times previously, the patient's new IgG values to shrimp or to pork are normalized to the mean of five-times test results to shrimp or the mean of seven-times test results to pork. The normalized value of the patient's IgG specific to shrimp can be 6.0 and the normalized value of the patient's IgG specific to pork can be 1.0. In this scenario, the patient has six times higher sensitivity to shrimp at this time compared to his average sensitivity to shrimp, but substantially similar sensitivity to pork. Then, the patient's sensitivity to shrimp and pork can be indexed based on such comparison.

Methodology to Determine the Subset of Psoriasis Patients with Food Sensitivities that Underlie Psoriasis:

While it is suspected that food sensitivities may play a substantial role in signs and symptoms of psoriasis, some psoriasis patients may not have food sensitivities that underlie psoriasis. Those patients may not be benefit from dietary intervention to treat signs and symptoms of psoriasis. To determine the subset of such patients, body fluid samples of psoriasis patients and non-psoriasis patients can be tested with ELISA test using test devices with 24 food samples.

Table 5A and Table 5B provide exemplary raw data. As should be readily appreciated, the data indicate number of positive results out of 90 sample foods based on 90^{th} percentile value (Table 5A) or 95^{th} percentile value (Table 5B). The first column is psoriasis (n=133); second column is non-psoriasis (n=240) by ICD-10 code. Average and median number of positive foods was computed for psoriasis and non-psoriasis patients. From the raw data shown in Table 5A and Table 5B, average and standard deviation of the number of positive foods was computed for psoriasis and non-psoriasis patients. Additionally, the number and percentage of patients with zero positive foods was calculated for both psoriasis and non-psoriasis. The number and percentage of patients with zero positive foods in the psoriasis population is almost half of the percentage of patients with zero positive foods in the non-psoriasis population (8.3% vs. 15.4%, respectively) based on 90^{th} percentile value (Table 5A), and this percentage is also half in the psoriasis population of that seen in the non-psoriasis population (16.5% vs. 35.0%, respectively) based on 95^{th} percentile value (Table 5B). Thus, it can be easily appreciated that the psoriasis patient having sensitivity to zero positive foods is unlikely to have food sensitivities underlying their signs and symptoms of psoriasis.

Table 6A and Table 7A show exemplary statistical data summarizing the raw data of two patient populations shown in Table 5A. The statistical data includes normality, arithmetic mean, median, percentiles and 95% confidence interval (CI) for the mean and median representing number of positive foods in the psoriasis population and the non-psoriasis population. Table 6B and Table 7B show exemplary statistical data summarizing the raw data of two patient populations shown in Table 5B. The statistical data includes normality, arithmetic mean, median, percentiles and 95% confidence interval (CI) for the mean and median representing number of positive foods in the psoriasis population and the non-psoriasis population.

Table 8A and Table 9A show exemplary statistical data summarizing the raw data of two patient populations shown in Table 5A. In Tables 8A and 9A, the raw data was transformed by logarithmic transformation to improve the data interpretation. Table 8B and Table 9B show another exemplary statistical data summarizing the raw data of two patient populations shown in Table 5B. In Tables 8B and 9B, the raw data was transformed by logarithmic transformation to improve the data interpretation.

Table 10A and Table 11A show exemplary statistical data of an independent T-test (Table 10A, logarithmically transformed data) and a Mann-Whitney test (Table 11A) to compare the geometric mean number of positive foods between the psoriasis and non-psoriasis samples. The data shown in Table 10A and Table 11A indicate statistically significant differences in the geometric mean of positive number of foods between the psoriasis population and the non-psoriasis population. In both statistical tests, it is shown that the number of positive responses with 90 food samples is significantly higher in the psoriasis population than in the non-psoriasis population with an average discriminatory p-value of ≤0.0001. These statistical data is also illustrated as a box and whisker plot in FIG. 6A, and a notched box and whisker plot in FIG. 6B.

Table 10B and Table 11B show exemplary statistical data of an independent T-test (Table 10A, logarithmically transformed data) and a Mann-Whitney test (Table 11B) to compare the geometric mean number of positive foods between the psoriasis and non-psoriasis samples. The data shown in Table 10B and Table 11B indicate statistically significant differences in the geometric mean of positive number of foods between the psoriasis population and the non-psoriasis population. In both statistical tests, it is shown that the number of positive responses with 90 food samples is significantly higher in the psoriasis population than in the non-psoriasis population with an average discriminatory p-value of ≤0.0001. These statistical data is also illustrated as a box and whisker plot in FIG. 6C, and a notched box and whisker plot in FIG. 6D.

Table 12A shows exemplary statistical data of a Receiver Operating Characteristic (ROC) curve analysis of data shown in Tables 5A-11A to determine the diagnostic power of the test used in Table 5 at discriminating psoriasis from non-psoriasis subjects. When a cutoff criterion of more than 5 positive foods is used, the test yields a data with 61.65% sensitivity and 64.17% specificity, with an area under the curve (AUROC) of 0.670. The p-value for the ROC is significant at a p-value of ≤0.0001. FIG. 7A illustrates the ROC curve corresponding to the statistical data shown in Table 12A. Because the statistical difference between the psoriasis population and the non-psoriasis population is significant when the test results are cut off to a positive number of 5, the number of foods for which a patient tests positive could be used as a confirmation of the primary clinical diagnosis of psoriasis, and whether it is likely that food sensitivities underlies on the patient's signs and symptoms of psoriasis. Therefore, the above test can be used as another ‘rule in’ test to add to currently available clinical criteria for diagnosis for psoriasis.

As shown in Tables 5A-12A, and FIG. 7A, based on 90^{th} percentile data, the number of positive foods seen in psoriasis vs. non-psoriasis subjects is significantly different whether the geometric mean or median of the data is compared. The number of positive foods that a person has is indicative of the presence of psoriasis in subjects. The test has discriminatory power to detect psoriasis with ˜62% sensitivity and ˜64% specificity. Additionally, the absolute number and percentage of subjects with 0 positive foods is also very different in psoriasis vs. non-psoriasis subjects, with a far lower percentage of psoriasis subjects (8.3%) having 0 positive foods than non-psoriasis subjects (15.4%). The data suggests a subset of psoriasis patients may have psoriasis due to other factors than diet, and may not benefit from dietary restriction.

Table 12B shows exemplary statistical data of a Receiver Operating Characteristic (ROC) curve analysis of data shown in Tables 5B-11B to determine the diagnostic power of the test used in Table 5 at discriminating psoriasis from non-psoriasis subjects. When a cutoff criterion of more than 6 positive foods is used, the test yields a data with 39.9% sensitivity and 86.3% specificity, with an area under the curve (AUROC) of 0.676. The p-value for the ROC is significant at a p-value of ≤0.0001. FIG. 7B illustrates the ROC curve corresponding to the statistical data shown in Table 12B. Because the statistical difference between the psoriasis population and the non-psoriasis population is significant when the test results are cut off to positive number of 6, the number of foods that a patient tests positive could be used as a confirmation of the primary clinical diagnosis of psoriasis, and whether it is likely that food sensitivities underlies on the patient's signs and symptoms of psoriasis. Therefore, the above test can be used as another ‘rule in’ test to add to currently available clinical criteria for diagnosis for psoriasis.

As shown in Tables 5B-12B, and FIG. 7B, based on 95^{th} percentile data, the number of positive foods seen in psoriasis vs. non-psoriasis subjects is significantly different whether the geometric mean or median of the data is compared. The number of positive foods that a person has is indicative of the presence of psoriasis in subjects. The test has discriminatory power to detect psoriasis with ˜40% sensitivity and ˜86% specificity. Additionally, the absolute number and percentage of subjects with 0 positive foods is also very different in psoriasis vs. non-psoriasis subjects, with a far lower percentage of psoriasis subjects (16.5%) having 0 positive foods than non-psoriasis subjects (35%). The data suggests a subset of psoriasis patients may have psoriasis due to other factors than diet, and may not benefit from dietary restriction.

Method for Determining Distribution of Per-Person Number of Foods Declared “Positive”:

To determine the distribution of number of “positive” foods per person and measure the diagnostic performance, the analysis was performed with 90 food items from the Table 1, which shows most positive responses to psoriasis patients. The 90 food items includes chocolate, grapefruit, honey, malt, rye, baker's yeast, brewer's yeast, broccoli, cola nut, tobacco, mustard, green pepper, buck wheat, avocado, cane sugar, cantaloupe, garlic, cucumber, cauliflower, sunflower seed, lemon, strawberry, eggplant, wheat, olive, halibut, cabbage, orange, rice, safflower, tomato, almond, oat, barley, peach, grape, potato, spinach, sole, and butter. To attenuate the influence of any one subject on this analysis, each food-specific and gender-specific dataset was bootstrap resampled 1000 times. Then, for each food item in the bootstrap sample, sex-specific cutpoint was determined using the 90th and 95th percentiles of the control population. Once the sex-specific cutpoints were determined, the sex-specific cutpoints was compared with the observed ELISA signal scores for both control and psoriasis subjects. In this comparison, if the observed signal is equal or more than the cutpoint value, then it is determined “positive” food, and if the observed signal is less than the cutpoint value, then it is determined “negative” food.

Once all food items were determined either positive or negative, the results of the 180 (90 foods×2 cutpoints) calls for each subject were saved within each bootstrap replicate. Then, for each subject, 90 calls were summed using 90^{th} percentile as cutpoint to get “Number of Positive Foods (90^{th}),” and the rest of 90 calls were summed using 95^{th} percentile to get “Number of Positive Foods (95^{th})” Then, within each replicate, “Number of Positive Foods (90^{th})” and “Number of Positive Foods (95^{th})” were summarized across subjects to get descriptive statistics for each replicate as follows: 1) overall means equals to the mean of means, 2) overall standard deviation equals to the mean of standard deviations, 3) overall medial equals to the mean of medians, 4) overall minimum equals to the minimum of minimums, and 5) overall maximum equals to maximum of maximum. In this analysis, to avoid non-integer “Number of Positive Foods” when computing frequency distribution and histogram, the authors pretended that the 1000 repetitions of the same original dataset were actually 999 sets of new subjects of the same size added to the original sample. Once the summarization of data is done, frequency distributions and histograms were generated for both “Number of Positive Foods. (90^{th})” and “Number of Positive Foods (95^{th})” for both genders and for both psoriasis subjects and control subjects using programs “a_pos_foods.sas, a_pos_foods_by_dx.sas”.

Method for Measuring Diagnostic Performance:

To measure diagnostic performance for each food items for each subject, we used data of “Number of Positive Foods (90^{th})” and “Number of Positive Foods (95^{th})” for each subject within each bootstrap replicate described above. In this analysis, the cutpoint was set to 1. Thus, if a subject has one or more “Number of Positive Foods (90^{th})”, then the subject is called “Has psoriasis.” If a subject has less than one “Number of Positive Foods (90^{th})”, then the subject is called “Does Not Have psoriasis.” When all calls were made, the calls were compared with actual diagnosis to determine whether a call was a True Positive (TP), True Negative (TN), False Positive (FP), or False Negative (FN). The comparisons were summarized across subjects to get the performance metrics of sensitivity, specificity, positive predictive value, and negative predictive value for both “Number of Positive Foods(90^{th})” and “Number of Positive Foods(95^{th})” when the cutpoint is set to 1 for each method. Each (sensitivity, 1-specificity) pair becomes a point on the ROC curve for this replicate.

To increase the accuracy, the analysis above was repeated by incrementing cutpoint from 2 up to 24, and repeated for each of the 1000 bootstrap replicates. Then the performance metrics across the 1000 bootstrap replicates were summarized by calculating averages using a program “t_pos_foods_by_dx.sas”. The results of diagnostic performance for female and male are shown in Table 13 (90^{th} percentile) and Table 14 (95^{th} percentile).

Of course, it should be appreciated that certain variations in the food preparations may be made without altering the general scope of the subject matter presented herein. For example, where the food item was yellow onion, that item should be understood to also include other onion varieties that were demonstrated to have equivalent activity in the tests. Indeed, the inventors have noted that for each tested food preparation, certain other related food preparations also tested in the same or equivalent manner (data not shown). Thus, it should be appreciated that each tested and claimed food preparation will have equivalent related preparations with demonstrated equal or equivalent reactions in the test.

It should be apparent to those skilled in the art that many more modifications besides those already described are possible without departing from the concepts herein. The subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

TABLE 1

Abalone
Adlay
Almond
American Cheese
Apple
Artichoke
Asparagus
Avocado
Baby Bok Choy
Bamboo shoots
Banana
Barley, whole grain
Beef
Beets
Beta-lactoglobulin
Blueberry
Broccoli
Buckwheat
Butter
Cabbage
Cane sugar
Cantaloupe
Caraway
Carrot
Casein
Cashew
Cauliflower
Celery
Chard
Cheddar Cheese
Chick Peas
Chicken
Chili pepper
Chocolate
Cinnamon
Clam
Cocoa Bean
Coconut
Codfish
Coffee
Cola nut
Corn
Cottage cheese
Cow's milk
Crab
Cucumber
Cured Cheese
Cuttlefish
Duck
Durian
Eel
Egg White (separate)
Egg Yolk (separate)
Egg, white/yolk (comb.)
Eggplant
Garlic
Ginger
Gluten - Gliadin
Goat's milk
Grape, white/concord
Grapefruit
Grass Carp
Green Onion
Green pea
Green pepper
Guava
Hair Tail
Hake
Halibut
Hazelnut
Honey
Kelp
Kidney bean
Kiwi Fruit
Lamb
Leek
Lemon
Lentils
Lettuce, Iceberg
Lima bean
Lobster
Longan
Mackerel
Malt
Mango
Marjoram
Millet
Mung bean
Mushroom
Mustard seed
Oat
Olive
Onion
Orange
Oyster
Papaya
Paprika
Parsley
Peach
Peanut
Pear
Pepper, Black
Pineapple
Pinto bean
Plum
Pork
Potato
Rabbit
Rice
Roquefort Cheese
Rye
Saccharine
Safflower seed
Salmon
Sardine
Scallop
Sesame
Shark fin
Sheep's milk
Shrimp
Sole
Soybean
Spinach
Squashes
Squid
Strawberry
String bean
Sunflower seed
Sweet potato
Swiss cheese
Taro
Tea, black
Tobacco
Tomato
Trout
Tuna
Turkey
Vanilla
Walnut, black
Watermelon
Welch Onion
Wheat
Wheat bran
Yeast (S. cerevisiae)
Yogurt
FOOD ADDITIVES
Arabic Gum
Carboxymethyl Cellulose
Carrageneenan
FD&C Blue #1
FD&C Red #3
FD&C Red #40
FD&C Yellow #5
FD&C Yellow #6
Gelatin
Guar Gum
Maltodextrin
Pectin
Whey
Xanthan Gum

TABLE 2
Ranking of Foods according to 2-tailed Permutation
T-test p-values with FDR adjustment

| | | | FDR |
|---|---|---|---|
| | | Raw | Multiplicity-adj |
| Rank | Food | p-value | p-value |
| 1 | Peach | 0.0000 | 0.0000 |
| 2 | Cucumber | 0.0000 | 0.0009 |
| 3 | Tea | 0.0000 | 0.0009 |
| 4 | Tomato | 0.0000 | 0.0009 |
| 5 | Broccoli | 0.0001 | 0.0009 |
| 6 | Cauliflower | 0.0001 | 0.0009 |
| 7 | Almond | 0.0001 | 0.0011 |
| 8 | Green_Pepper | 0.0001 | 0.0011 |
| 9 | Grapefruit | 0.0001 | 0.0013 |
| 10 | Tobacco | 0.0001 | 0.0013 |
| 11 | Eggplant | 0.0002 | 0.0013 |
| 12 | Rye | 0.0003 | 0.0023 |
| 13 | Oat | 0.0003 | 0.0024 |
| 14 | Cantaloupe | 0.0004 | 0.0024 |
| 15 | Cabbage | 0.0004 | 0.0024 |
| 16 | Cane_Sugar | 0.0005 | 0.0029 |
| 17 | Sweet_Pot_{—} | 0.0005 | 0.0029 |
| 18 | Pineapple | 0.0006 | 0.0029 |
| 19 | Avocado | 0.0008 | 0.0035 |
| 20 | Orange | 0.0008 | 0.0035 |
| 21 | Spinach | 0.0008 | 0.0035 |
| 22 | Honey | 0.0009 | 0.0038 |
| 23 | Swiss_Ch_{—} | 0.0012 | 0.0048 |
| 24 | Malt | 0.0013 | 0.0048 |
| 25 | Mustard | 0.0013 | 0.0048 |
| 26 | Wheat | 0.0017 | 0.0060 |
| 27 | Apple | 0.0020 | 0.0065 |
| 28 | Chocolate | 0.0020 | 0.0065 |
| 29 | Yogurt | 0.0021 | 0.0065 |
| 30 | Goat_Milk | 0.0022 | 0.0065 |
| 31 | Cola_Nut | 0.0023 | 0.0067 |
| 32 | Clam | 0.0024 | 0.0067 |
| 33 | Cheddar_Ch_{—} | 0.0024 | 0.0067 |
| 34 | Olive | 0.0031 | 0.0083 |
| 35 | Yeast_Brewer | 0.0033 | 0.0084 |
| 36 | Butter | 0.0038 | 0.0095 |
| 37 | Celery | 0.0039 | 0.0095 |
| 38 | Onion | 0.0041 | 0.0097 |
| 39 | Garlic | 0.0048 | 0.0112 |
| 40 | Walnut_Blk | 0.0053 | 0.0118 |
| 41 | Cottage_Ch_{—} | 0.0056 | 0.0121 |
| 42 | Yeast_Baker | 0.0057 | 0.0121 |
| 43 | Cow_Milk | 0.0059 | 0.0123 |
| 44 | Corn | 0.0066 | 0.0136 |
| 45 | Amer_Cheese | 0.0069 | 0.0137 |
| 46 | Strawberry | 0.0070 | 0.0137 |
| 47 | Buck_Wheat | 0.0071 | 0.0137 |
| 48 | Lemon | 0.0131 | 0.0245 |
| 49 | Green_Pea | 0.0190 | 0.0348 |
| 50 | Trout | 0.0200 | 0.0356 |
| 51 | Barley | 0.0202 | 0.0356 |
| 52 | Potato | 0.0206 | 0.0356 |
| 53 | Beef | 0.0223 | 0.0379 |
| 54 | Rice | 0.0227 | 0.0379 |
| 55 | Sunflower_Sd | 0.0248 | 0.0405 |
| 56 | Chili_Pepper | 0.0293 | 0.0472 |
| 57 | Banana | 0.0343 | 0.0542 |
| 58 | String_Bean | 0.0429 | 0.0655 |
| 59 | Safflower | 0.0429 | 0.0655 |
| 60 | Pinto_Bean | 0.0755 | 0.1133 |
| 61 | Cinnamon | 0.0962 | 0.1420 |
| 62 | Lima_Bean | 0.0987 | 0.1433 |
| 63 | Parsley | 0.1043 | 0.1490 |
| 64 | Shrimp | 0.1091 | 0.1534 |
| 65 | Squashes | 0.1350 | 0.1870 |
| 66 | Blueberry | 0.1543 | 0.2104 |
| 67 | Coffee | 0.1830 | 0.2458 |
| 68 | Tuna | 0.1920 | 0.2541 |
| 69 | Carrot | 0.2066 | 0.2695 |
| 70 | Sardine | 0.2145 | 0.2758 |
| 71 | Mushroom | 0.2268 | 0.2875 |
| 72 | Peanut | 0.3606 | 0.4477 |
| 73 | Codfish | 0.3631 | 0.4477 |
| 74 | Lobster | 0.3737 | 0.4545 |
| 75 | Halibut | 0.3928 | 0.4714 |
| 76 | Millet | 0.4224 | 0.5002 |
| 77 | Pork | 0.4461 | 0.5214 |
| 78 | Oyster | 0.4730 | 0.5457 |
| 79 | Turkey | 0.4958 | 0.5649 |
| 80 | Grape | 0.5046 | 0.5677 |
| 81 | Scallop | 0.6187 | 0.6808 |
| 82 | Salmon | 0.6203 | 0.6808 |
| 83 | Lettuce | 0.6583 | 0.7138 |
| 84 | Chicken | 0.7193 | 0.7707 |
| 85 | Egg | 0.7671 | 0.8122 |
| 86 | Crab | 0.7781 | 0.8143 |
| 87 | Soybean | 0.7932 | 0.8206 |
| 88 | Sole | 0.8287 | 0.8393 |
| 89 | Sesame | 0.8300 | 0.8393 |
| 90 | Cashew | 0.8677 | 0.8677 |

TABLE 3
Basic Descriptive Statistics of ELISA Score by Food and Gender
Comparing Psoriasis to Control

| | | | ELISA Score | | | | |
|---|---|---|---|---|---|---|---|
| Sex | Food | Diagnosis | N | Mean | SD | Min | Max |
| FEMALE | Almond | Psoriasis | 66 | 9.463 | 25.099 | 0.100 | 196.38 |
| | | Control | 120 | 4.382 | 3.344 | 0.100 | 26.669 |
| | | Diff (1 − 2) | — | 5.081 | 15.158 | — | — |
| | Amer_Cheese | Psoriasis | 66 | 38.439 | 76.854 | 0.100 | 400.00 |
| | | Control | 120 | 27.290 | 48.298 | 1.113 | 229.42 |
| | | Diff (1 − 2) | — | 11.149 | 59.960 | — | — |
| | Apple | Psoriasis | 66 | 10.134 | 22.758 | 0.100 | 164.02 |
| | | Control | 120 | 4.925 | 5.686 | 0.100 | 47.698 |
| | | Diff (1 − 2) | — | 5.209 | 14.279 | — | — |
| | Avocado | Psoriasis | 66 | 7.702 | 27.594 | 0.100 | 223.45 |
| | | Control | 120 | 2.928 | 4.389 | 0.100 | 44.515 |
| | | Diff (1 − 2) | — | 4.774 | 16.776 | — | — |
| | Banana | Psoriasis | 66 | 18.803 | 39.094 | 0.100 | 230.22 |
| | | Control | 120 | 7.410 | 25.928 | 0.100 | 282.41 |
| | | Diff (1 − 2) | — | 11.393 | 31.220 | — | — |
| | Barley | Psoriasis | 66 | 28.561 | 37.864 | 3.612 | 289.39 |
| | | Control | 120 | 23.262 | 16.540 | 4.506 | 85.580 |
| | | Diff (1 − 2) | — | 5.299 | 26.142 | — | — |
| | Beef | Psoriasis | 66 | 13.668 | 26.586 | 0.391 | 194.86 |
| | | Control | 120 | 8.730 | 5.391 | 1.236 | 33.732 |
| | | Diff (1 − 2) | — | 4.938 | 16.386 | — | — |
| | Blueberry | Psoriasis | 66 | 6.911 | 9.658 | 0.100 | 62.336 |
| | | Control | 120 | 6.109 | 5.322 | 0.100 | 37.312 |
| | | Diff (1 − 2) | — | 0.802 | 7.160 | — | — |
| | Broccoli | Psoriasis | 66 | 15.344 | 33.026 | 0.100 | 207.16 |
| | | Control | 120 | 6.331 | 6.550 | 0.100 | 66.265 |
| | | Diff (1 − 2) | — | 9.013 | 20.324 | — | — |
| | Buck_Wheat | Psoriasis | 66 | 15.287 | 26.424 | 2.125 | 170.89 |
| | | Control | 120 | 8.413 | 5.866 | 0.247 | 48.998 |
| | | Diff (1 − 2) | — | 6.873 | 16.398 | — | — |
| | Butter | Psoriasis | 66 | 35.022 | 56.419 | 1.593 | 357.31 |
| | | Control | 120 | 21.399 | 23.407 | 1.686 | 120.98 |
| | | Diff (1 − 2) | — | 13.623 | 38.455 | — | — |
| | Cabbage | Psoriasis | 66 | 15.924 | 35.280 | 0.100 | 236.14 |
| | | Control | 120 | 6.414 | 10.430 | 0.100 | 96.832 |
| | | Diff (1 − 2) | — | 9.509 | 22.585 | — | — |
| | Cane_Sugar | Psoriasis | 66 | 31.243 | 35.380 | 6.143 | 275.59 |
| | | Control | 120 | 25.083 | 30.963 | 5.114 | 246.06 |
| | | Diff (1 − 2) | — | 6.159 | 32.592 | — | — |
| | Cantaloupe | Psoriasis | 66 | 16.024 | 41.224 | 0.100 | 298.22 |
| | | Control | 120 | 6.106 | 4.312 | 1.253 | 35.519 |
| | | Diff (1 − 2) | — | 9.917 | 24.746 | — | — |
| | Carrot | Psoriasis | 66 | 9.735 | 19.785 | 0.100 | 112.40 |
| | | Control | 120 | 6.626 | 10.376 | 0.100 | 81.659 |
| | | Diff (1 − 2) | — | 3.109 | 14.419 | — | — |
| | Cashew | Psoriasis | 66 | 15.343 | 31.364 | 0.100 | 238.59 |
| | | Control | 120 | 15.596 | 24.671 | 0.100 | 115.05 |
| | | Diff (1 − 2) | — | −0.253 | 27.224 | — | — |
| | Cauliflower | Psoriasis | 66 | 13.156 | 29.717 | 0.100 | 192.10 |
| | | Control | 120 | 4.439 | 4.040 | 0.100 | 34.046 |
| | | Diff (1 − 2) | — | 8.717 | 17.959 | — | — |
| | Celery | Psoriasis | 66 | 17.121 | 35.082 | 2.443 | 273.52 |
| | | Control | 120 | 11.433 | 9.083 | 2.967 | 63.628 |
| | | Diff (1 − 2) | — | 5.688 | 22.094 | — | — |
| | Cheddar_Ch_{—} | Psoriasis | 66 | 47.106 | 86.527 | 1.308 | 400.00 |
| | | Control | 120 | 34.129 | 61.341 | 0.614 | 400.00 |
| | | Diff (1 − 2) | — | 12.977 | 71.263 | — | — |
| | Chicken | Psoriasis | 66 | 25.858 | 49.683 | 3.260 | 367.76 |
| | | Control | 120 | 22.187 | 18.930 | 5.601 | 128.81 |
| | | Diff (1 − 2) | — | 3.671 | 33.223 | — | — |
| | Chili_Pepper | Psoriasis | 66 | 12.077 | 18.324 | 0.100 | 108.67 |
| | | Control | 120 | 9.522 | 10.042 | 0.244 | 66.696 |
| | | Diff (1 − 2) | — | 2.555 | 13.558 | — | — |
| | Chocolate | Psoriasis | 66 | 25.088 | 34.270 | 4.555 | 273.65 |
| | | Control | 120 | 17.776 | 11.393 | 3.160 | 80.219 |
| | | Diff (1 − 2) | — | 7.312 | 22.334 | — | — |
| | Cinnamon | Psoriasis | 66 | 46.046 | 36.230 | 8.411 | 229.67 |
| | | Control | 120 | 41.665 | 27.573 | 3.555 | 141.66 |
| | | Diff (1 − 2) | — | 4.380 | 30.909 | — | — |
| | Clam | Psoriasis | 66 | 56.705 | 58.927 | 7.862 | 370.14 |
| | | Control | 120 | 43.165 | 25.445 | 8.396 | 162.89 |
| | | Diff (1 − 2) | — | 13.540 | 40.563 | — | — |
| | Codfish | Psoriasis | 66 | 20.807 | 28.255 | 2.087 | 224.29 |
| | | Control | 120 | 34.172 | 41.473 | 5.844 | 319.60 |
| | | Diff (1 − 2) | — | −13.365 | 37.342 | — | — |
| | Coffee | Psoriasis | 66 | 30.135 | 41.476 | 0.130 | 219.47 |
| | | Control | 120 | 29.592 | 45.077 | 4.151 | 400.00 |
| | | Diff (1 − 2) | — | 0.543 | 43.839 | — | — |
| | Cola_Nut | Psoriasis | 66 | 41.054 | 30.225 | 14.161 | 253.15 |
| | | Control | 120 | 35.040 | 17.705 | 9.514 | 115.41 |
| | | Diff (1 − 2) | — | 6.014 | 22.923 | — | — |
| | Corn | Psoriasis | 66 | 26.999 | 62.011 | 0.100 | 400.00 |
| | | Control | 120 | 11.069 | 12.512 | 0.975 | 84.673 |
| | | Diff (1 − 2) | — | 15.930 | 38.206 | — | — |
| | Cottage_Ch_{—} | Psoriasis | 66 | 92.936 | 128.492 | 2.972 | 400.00 |
| | | Control | 120 | 85.171 | 110.987 | 2.680 | 400.00 |
| | | Diff (1 − 2) | — | 7.765 | 117.469 | — | — |
| | Cow_Milk | Psoriasis | 66 | 88.109 | 123.113 | 1.427 | 400.00 |
| | | Control | 120 | 82.324 | 106.893 | 1.527 | 400.00 |
| | | Diff (1 − 2) | — | 5.785 | 112.889 | — | — |
| | Crab | Psoriasis | 66 | 22.569 | 22.755 | 2.916 | 114.91 |
| | | Control | 120 | 23.975 | 16.743 | 3.654 | 98.750 |
| | | Diff (1 − 2) | — | −1.405 | 19.084 | — | — |
| | Cucumber | Psoriasis | 66 | 21.399 | 46.134 | 1.806 | 238.43 |
| | | Control | 120 | 8.249 | 7.926 | 0.382 | 54.906 |
| | | Diff (1 − 2) | — | 13.150 | 28.151 | — | — |
| | Egg | Psoriasis | 66 | 50.720 | 63.917 | 0.125 | 312.95 |
| | | Control | 120 | 43.188 | 72.783 | 0.100 | 400.00 |
| | | Diff (1 − 2) | — | 7.532 | 69.780 | — | — |
| | Eggplant | Psoriasis | 66 | 13.670 | 29.480 | 0.100 | 215.30 |
| | | Control | 120 | 5.983 | 7.662 | 0.731 | 69.612 |
| | | Diff (1 − 2) | — | 7.687 | 18.573 | — | — |
| | Garlic | Psoriasis | 66 | 21.794 | 43.623 | 3.814 | 325.24 |
| | | Control | 120 | 14.822 | 16.638 | 0.194 | 126.94 |
| | | Diff (1 − 2) | — | 6.972 | 29.177 | — | — |
| | Goat_Milk | Psoriasis | 66 | 26.737 | 60.846 | 0.783 | 400.00 |
| | | Control | 120 | 15.468 | 29.678 | 0.705 | 200.19 |
| | | Diff (1 − 2) | — | 11.269 | 43.330 | — | — |
| | Grape | Psoriasis | 66 | 24.055 | 28.219 | 7.119 | 219.77 |
| | | Control | 120 | 23.342 | 8.740 | 0.242 | 65.157 |
| | | Diff (1 − 2) | — | 0.713 | 18.185 | — | — |
| | Grapefruit | Psoriasis | 66 | 8.884 | 26.747 | 0.100 | 192.11 |
| | | Control | 120 | 3.242 | 2.505 | 0.100 | 15.775 |
| | | Diff (1 − 2) | — | 5.642 | 16.024 | — | — |
| | Green_Pea | Psoriasis | 66 | 17.339 | 19.594 | 0.561 | 91.663 |
| | | Control | 120 | 12.270 | 16.744 | 0.100 | 103.64 |
| | | Diff (1 − 2) | — | 5.069 | 17.803 | — | — |
| | Green_Pepper | Psoriasis | 66 | 11.397 | 28.112 | 0.100 | 179.23 |
| | | Control | 120 | 4.146 | 3.731 | 0.087 | 30.934 |
| | | Diff (1 − 2) | — | 7.251 | 16.976 | — | — |
| | Halibut | Psoriasis | 66 | 12.959 | 16.112 | 2.087 | 131.03 |
| | | Control | 120 | 17.087 | 37.388 | 0.167 | 369.33 |
| | | Diff (1 − 2) | — | −4.128 | 31.556 | — | — |
| | Honey | Psoriasis | 66 | 18.555 | 34.347 | 4.241 | 273.98 |
| | | Control | 120 | 11.291 | 6.987 | 0.112 | 50.000 |
| | | Diff (1 − 2) | — | 7.264 | 21.174 | — | — |
| | Lemon | Psoriasis | 66 | 7.138 | 28.085 | 0.100 | 229.12 |
| | | Control | 120 | 2.781 | 3.856 | 0.078 | 39.087 |
| | | Diff (1 − 2) | — | 4.357 | 16.978 | — | — |
| | Lettuce | Psoriasis | 66 | 15.696 | 22.580 | 0.261 | 131.33 |
| | | Control | 120 | 15.614 | 19.484 | 0.201 | 143.66 |
| | | Diff (1 − 2) | — | 0.083 | 20.631 | — | — |
| | Lima_Bean | Psoriasis | 66 | 11.588 | 17.657 | 0.100 | 123.34 |
| | | Control | 120 | 7.890 | 7.515 | 0.100 | 50.711 |
| | | Diff (1 − 2) | — | 3.699 | 12.110 | — | — |
| | Lobster | Psoriasis | 66 | 15.344 | 13.116 | 1.984 | 87.594 |
| | | Control | 120 | 16.677 | 12.421 | 0.289 | 68.024 |
| | | Diff (1 − 2) | — | −1.333 | 12.671 | — | — |
| | Malt | Psoriasis | 66 | 32.965 | 42.853 | 8.078 | 352.20 |
| | | Control | 120 | 24.523 | 13.672 | 0.464 | 81.685 |
| | | Diff (1 − 2) | — | 8.442 | 27.742 | — | — |
| | Millet | Psoriasis | 66 | 9.865 | 41.132 | 0.100 | 336.61 |
| | | Control | 120 | 4.114 | 3.796 | 0.084 | 29.570 |
| | | Diff (1 − 2) | — | 5.752 | 24.637 | — | — |
| | Mushroom | Psoriasis | 66 | 11.738 | 17.723 | 0.100 | 103.71 |
| | | Control | 120 | 15.108 | 20.203 | 0.100 | 116.91 |
| | | Diff (1 − 2) | — | −3.369 | 19.363 | — | — |
| | Mustard | Psoriasis | 66 | 15.951 | 33.513 | 0.130 | 254.66 |
| | | Control | 120 | 8.930 | 5.327 | 0.113 | 31.013 |
| | | Diff (1 − 2) | — | 7.021 | 20.374 | — | — |
| | Oat | Psoriasis | 66 | 30.354 | 36.254 | 1.346 | 221.54 |
| | | Control | 120 | 23.470 | 36.732 | 0.125 | 290.37 |
| | | Diff (1 − 2) | — | 6.883 | 36.564 | — | — |
| | Olive | Psoriasis | 66 | 36.086 | 41.158 | 3.253 | 275.98 |
| | | Control | 120 | 26.615 | 22.584 | 0.254 | 182.46 |
| | | Diff (1 − 2) | — | 9.471 | 30.468 | — | — |
| | Onion | Psoriasis | 66 | 28.282 | 73.025 | 0.100 | 400.00 |
| | | Control | 120 | 12.851 | 15.238 | 0.240 | 95.689 |
| | | Diff (1 − 2) | — | 15.431 | 45.100 | — | — |
| | Orange | Psoriasis | 66 | 37.397 | 53.101 | 2.355 | 315.04 |
| | | Control | 120 | 21.610 | 24.737 | 0.100 | 144.76 |
| | | Diff (1 − 2) | — | 15.787 | 37.307 | — | — |
| | Oyster | Psoriasis | 66 | 59.961 | 61.669 | 7.438 | 400.00 |
| | | Control | 120 | 69.943 | 81.247 | 0.524 | 400.00 |
| | | Diff (1 − 2) | — | −9.982 | 74.917 | — | — |
| | Parsley | Psoriasis | 66 | 7.608 | 15.306 | 0.100 | 96.051 |
| | | Control | 120 | 8.922 | 18.491 | 0.100 | 115.44 |
| | | Diff (1 − 2) | — | −1.314 | 17.432 | — | — |
| | Peach | Psoriasis | 66 | 20.149 | 45.657 | 0.100 | 288.45 |
| | | Control | 120 | 7.863 | 7.349 | 0.133 | 41.809 |
| | | Diff (1 − 2) | — | 12.285 | 27.773 | — | — |
| | Peanut | Psoriasis | 66 | 9.591 | 28.734 | 0.100 | 232.15 |
| | | Control | 120 | 4.997 | 5.150 | 0.071 | 30.134 |
| | | Diff (1 − 2) | — | 4.594 | 17.573 | — | — |
| | Pineapple | Psoriasis | 66 | 40.071 | 61.790 | 0.100 | 364.63 |
| | | Control | 120 | 22.992 | 46.848 | 0.191 | 400.00 |
| | | Diff (1 − 2) | — | 17.078 | 52.613 | — | — |
| | Pinto_Bean | Psoriasis | 66 | 14.230 | 25.699 | 1.897 | 180.65 |
| | | Control | 120 | 11.023 | 13.228 | 0.109 | 134.99 |
| | | Diff (1 − 2) | — | 3.207 | 18.614 | — | — |
| | Pork | Psoriasis | 66 | 16.094 | 21.829 | 0.100 | 134.28 |
| | | Control | 120 | 17.068 | 13.794 | 0.204 | 109.18 |
| | | Diff (1 − 2) | — | −0.974 | 17.070 | — | — |
| | Potato | Psoriasis | 66 | 23.808 | 51.516 | 3.926 | 376.25 |
| | | Control | 120 | 13.913 | 5.970 | 0.205 | 45.985 |
| | | Diff (1 − 2) | — | 9.894 | 30.993 | — | — |
| | Rice | Psoriasis | 66 | 29.880 | 42.091 | 4.972 | 279.93 |
| | | Control | 120 | 23.480 | 19.047 | 0.153 | 114.70 |
| | | Diff (1 − 2) | — | 6.400 | 29.334 | — | — |
| | Rye | Psoriasis | 66 | 10.086 | 24.836 | 0.100 | 205.41 |
| | | Control | 120 | 5.638 | 4.657 | 0.100 | 40.915 |
| | | Diff (1 − 2) | — | 4.448 | 15.229 | — | — |
| | Safflower | Psoriasis | 66 | 13.816 | 23.952 | 0.100 | 173.19 |
| | | Control | 120 | 9.930 | 10.477 | 0.100 | 87.082 |
| | | Diff (1 − 2) | — | 3.886 | 16.542 | — | — |
| | Salmon | Psoriasis | 66 | 11.326 | 14.055 | 0.100 | 98.129 |
| | | Control | 120 | 13.367 | 19.859 | 0.206 | 175.07 |
| | | Diff (1 − 2) | — | −2.041 | 18.024 | — | — |
| | Sardine | Psoriasis | 66 | 42.916 | 20.268 | 14.274 | 106.56 |
| | | Control | 120 | 41.394 | 23.930 | 0.531 | 179.66 |
| | | Diff (1 − 2) | — | 1.522 | 22.704 | — | — |
| | Scallop | Psoriasis | 66 | 72.160 | 28.995 | 11.905 | 152.42 |
| | | Control | 120 | 72.930 | 38.248 | 0.496 | 216.59 |
| | | Diff (1 − 2) | — | −0.770 | 35.258 | — | — |
| | Sesame | Psoriasis | 66 | 68.207 | 99.839 | 1.700 | 400.00 |
| | | Control | 120 | 75.917 | 93.152 | 0.432 | 400.00 |
| | | Diff (1 − 2) | — | −7.710 | 95.568 | — | — |
| | Shrimp | Psoriasis | 66 | 24.896 | 23.489 | 2.891 | 108.11 |
| | | Control | 120 | 40.662 | 33.157 | 0.173 | 145.07 |
| | | Diff (1 − 2) | — | −15.766 | 30.098 | — | — |
| | Sole | Psoriasis | 66 | 7.921 | 14.411 | 0.100 | 119.68 |
| | | Control | 120 | 5.802 | 4.249 | 0.100 | 43.730 |
| | | Diff (1 − 2) | — | 2.119 | 9.222 | — | — |
| | Soybean | Psoriasis | 66 | 22.020 | 26.692 | 1.304 | 191.68 |
| | | Control | 120 | 22.789 | 32.894 | 0.239 | 328.71 |
| | | Diff (1 − 2) | — | −0.768 | 30.846 | — | — |
| | Spinach | Psoriasis | 66 | 28.675 | 54.265 | 4.599 | 400.00 |
| | | Control | 120 | 18.031 | 11.903 | 0.349 | 81.566 |
| | | Diff (1 − 2) | — | 10.644 | 33.644 | — | — |
| | Squashes | Psoriasis | 66 | 17.779 | 16.726 | 1.435 | 99.530 |
| | | Control | 120 | 15.409 | 13.919 | 0.224 | 86.718 |
| | | Diff (1 − 2) | — | 2.369 | 14.971 | — | — |
| | Strawberry | Psoriasis | 66 | 9.497 | 13.259 | 0.100 | 67.954 |
| | | Control | 120 | 5.623 | 6.982 | 0.094 | 60.225 |
| | | Diff (1 − 2) | — | 3.874 | 9.676 | — | — |
| | String_Bean | Psoriasis | 66 | 54.297 | 36.746 | 13.372 | 206.67 |
| | | Control | 120 | 45.877 | 28.346 | 0.655 | 197.63 |
| | | Diff (1 − 2) | — | 8.420 | 31.570 | — | — |
| | Sunflower_Sd | Psoriasis | 66 | 16.896 | 26.382 | 2.916 | 171.20 |
| | | Control | 120 | 11.856 | 9.297 | 0.237 | 61.393 |
| | | Diff (1 − 2) | — | 5.040 | 17.372 | — | — |
| | Sweet_Pot_{—} | Psoriasis | 66 | 16.568 | 34.990 | 0.100 | 268.63 |
| | | Control | 120 | 8.661 | 6.190 | 0.126 | 53.190 |
| | | Diff (1 − 2) | — | 7.907 | 21.384 | — | — |
| | Swiss_Ch_{—} | Psoriasis | 66 | 61.885 | 107.888 | 0.874 | 400.00 |
| | | Control | 120 | 45.126 | 83.628 | 1.123 | 400.00 |
| | | Diff (1 − 2) | — | 16.759 | 92.925 | — | — |
| | Tea | Psoriasis | 66 | 38.989 | 21.305 | 11.118 | 123.92 |
| | | Control | 120 | 32.549 | 14.001 | 0.416 | 69.233 |
| | | Diff (1 − 2) | — | 6.440 | 16.944 | — | — |
| | Tobacco | Psoriasis | 66 | 55.956 | 51.658 | 7.519 | 271.30 |
| | | Control | 120 | 37.198 | 21.613 | 0.941 | 103.98 |
| | | Diff (1 − 2) | — | 18.758 | 35.282 | — | — |
| | Tomato | Psoriasis | 66 | 23.495 | 39.860 | 1.826 | 213.98 |
| | | Control | 120 | 9.746 | 8.861 | 0.208 | 60.077 |
| | | Diff (1 − 2) | — | 13.749 | 24.740 | — | — |
| | Trout | Psoriasis | 66 | 16.409 | 12.081 | 2.869 | 60.118 |
| | | Control | 120 | 20.268 | 21.381 | 0.166 | 187.12 |
| | | Diff (1 − 2) | — | −3.859 | 18.634 | — | — |
| | Tuna | Psoriasis | 66 | 18.887 | 31.419 | 3.499 | 244.00 |
| | | Control | 120 | 23.332 | 22.724 | 0.137 | 174.88 |
| | | Diff (1 − 2) | — | −4.445 | 26.128 | — | — |
| | Turkey | Psoriasis | 66 | 22.513 | 49.855 | 2.608 | 400.00 |
| | | Control | 120 | 15.406 | 10.344 | 0.297 | 70.688 |
| | | Diff (1 − 2) | — | 7.107 | 30.777 | — | — |
| | Walnut_Blk | Psoriasis | 66 | 37.778 | 48.751 | 6.591 | 385.96 |
| | | Control | 120 | 27.327 | 17.653 | 0.743 | 95.666 |
| | | Diff (1 − 2) | — | 10.451 | 32.266 | — | — |
| | Wheat | Psoriasis | 66 | 20.178 | 20.734 | 0.652 | 119.40 |
| | | Control | 120 | 18.041 | 20.533 | 0.372 | 128.56 |
| | | Diff (1 − 2) | — | 2.138 | 20.604 | — | — |
| | Yeast_Baker | Psoriasis | 66 | 13.228 | 24.840 | 1.814 | 185.88 |
| | | Control | 120 | 6.411 | 6.010 | 0.071 | 48.346 |
| | | Diff (1 − 2) | — | 6.818 | 15.535 | — | — |
| | Yeast_Brewer | Psoriasis | 66 | 23.808 | 31.963 | 2.996 | 149.52 |
| | | Control | 120 | 12.828 | 11.230 | 0.076 | 70.528 |
| | | Diff (1 − 2) | — | 10.980 | 21.035 | — | — |
| | Yogurt | Psoriasis | 66 | 31.284 | 58.867 | 2.775 | 400.00 |
| | | Control | 120 | 22.138 | 24.995 | 0.294 | 145.59 |
| | | Diff (1 − 2) | — | 9.146 | 40.351 | — | — |
| MALE | Almond | Psoriasis | 67 | 14.045 | 26.275 | 0.740 | 191.35 |
| | | Control | 120 | 4.515 | 4.047 | 0.100 | 26.332 |
| | | Diff (1 − 2) | — | 9.530 | 16.026 | — | — |
| | Amer_Cheese | Psoriasis | 67 | 49.902 | 77.319 | 0.740 | 400.00 |
| | | Control | 120 | 21.244 | 26.891 | 0.100 | 182.23 |
| | | Diff (1 − 2) | — | 28.658 | 50.970 | — | — |
| | Apple | Psoriasis | 67 | 11.904 | 16.364 | 0.529 | 74.230 |
| | | Control | 120 | 5.841 | 9.488 | 0.539 | 94.469 |
| | | Diff (1 − 2) | — | 6.063 | 12.387 | — | — |
| | Avocado | Psoriasis | 67 | 8.664 | 17.243 | 0.100 | 104.06 |
| | | Control | 120 | 2.613 | 1.676 | 0.100 | 12.006 |
| | | Diff (1 − 2) | — | 6.051 | 10.386 | — | — |
| | Banana | Psoriasis | 67 | 8.229 | 11.765 | 1.050 | 77.936 |
| | | Control | 120 | 6.805 | 17.738 | 0.100 | 181.50 |
| | | Diff (1 − 2) | — | 1.425 | 15.867 | — | — |
| | Barley | Psoriasis | 67 | 38.072 | 56.165 | 2.327 | 400.00 |
| | | Control | 120 | 23.373 | 17.951 | 5.215 | 119.95 |
| | | Diff (1 − 2) | — | 14.699 | 36.506 | — | — |
| | Beef | Psoriasis | 67 | 13.222 | 18.910 | 1.164 | 135.30 |
| | | Control | 120 | 8.724 | 9.515 | 0.100 | 81.880 |
| | | Diff (1 − 2) | — | 4.498 | 13.631 | — | — |
| | Blueberry | Psoriasis | 67 | 7.215 | 8.717 | 0.264 | 64.520 |
| | | Control | 120 | 5.492 | 5.759 | 0.100 | 39.800 |
| | | Diff (1 − 2) | — | 1.723 | 6.960 | — | — |
| | Broccoli | Psoriasis | 67 | 11.231 | 9.356 | 1.161 | 40.101 |
| | | Control | 120 | 5.868 | 4.685 | 0.100 | 29.187 |
| | | Diff (1 − 2) | — | 5.363 | 6.734 | — | — |
| | Buck_Wheat | Psoriasis | 67 | 11.351 | 8.711 | 1.164 | 41.385 |
| | | Control | 120 | 8.628 | 9.970 | 0.100 | 102.45 |
| | | Diff (1 − 2) | — | 2.724 | 9.540 | — | — |
| | Butter | Psoriasis | 67 | 37.269 | 41.773 | 1.375 | 167.04 |
| | | Control | 120 | 24.158 | 23.089 | 2.552 | 168.48 |
| | | Diff (1 − 2) | — | 13.110 | 31.072 | — | — |
| | Cabbage | Psoriasis | 67 | 13.084 | 17.139 | 0.846 | 82.785 |
| | | Control | 120 | 5.873 | 6.959 | 0.100 | 43.990 |
| | | Diff (1 − 2) | — | 7.211 | 11.660 | — | — |
| | Cane_Sugar | Psoriasis | 67 | 45.190 | 49.583 | 4.562 | 261.48 |
| | | Control | 120 | 21.755 | 17.953 | 3.067 | 153.43 |
| | | Diff (1 − 2) | — | 23.435 | 32.930 | — | — |
| | Cantaloupe | Psoriasis | 67 | 11.165 | 11.031 | 0.132 | 54.102 |
| | | Control | 120 | 6.149 | 4.629 | 0.100 | 38.586 |
| | | Diff (1 − 2) | — | 5.016 | 7.563 | — | — |
| | Carrot | Psoriasis | 67 | 7.089 | 6.305 | 0.132 | 32.623 |
| | | Control | 120 | 6.514 | 8.763 | 0.100 | 54.468 |
| | | Diff (1 − 2) | — | 0.575 | 7.974 | — | — |
| | Cashew | Psoriasis | 67 | 14.926 | 17.740 | 1.058 | 87.711 |
| | | Control | 120 | 13.751 | 25.310 | 0.100 | 191.59 |
| | | Diff (1 − 2) | — | 1.175 | 22.898 | — | — |
| | Cauliflower | Psoriasis | 67 | 9.482 | 9.827 | 0.846 | 40.723 |
| | | Control | 120 | 4.800 | 4.866 | 0.100 | 37.593 |
| | | Diff (1 − 2) | — | 4.682 | 7.049 | — | — |
| | Celery | Psoriasis | 67 | 19.699 | 23.303 | 1.481 | 138.45 |
| | | Control | 120 | 10.547 | 9.546 | 1.381 | 62.991 |
| | | Diff (1 − 2) | — | 9.152 | 15.886 | — | — |
| | Cheddar_Ch_{—} | Psoriasis | 67 | 64.247 | 94.164 | 0.815 | 396.18 |
| | | Control | 120 | 24.524 | 27.428 | 1.442 | 140.19 |
| | | Diff (1 − 2) | — | 39.723 | 60.392 | — | — |
| | Chicken | Psoriasis | 67 | 20.655 | 15.804 | 3.251 | 98.710 |
| | | Control | 120 | 21.525 | 14.252 | 4.785 | 72.374 |
| | | Diff (1 − 2) | — | −0.871 | 14.824 | — | — |
| | Chili_Pepper | Psoriasis | 67 | 15.269 | 19.184 | 0.925 | 113.06 |
| | | Control | 120 | 10.014 | 10.722 | 0.972 | 66.659 |
| | | Diff (1 − 2) | — | 5.255 | 14.326 | — | — |
| | Chocolate | Psoriasis | 67 | 21.566 | 11.727 | 3.148 | 63.694 |
| | | Control | 120 | 15.666 | 9.099 | 0.686 | 49.767 |
| | | Diff (1 − 2) | — | 5.900 | 10.115 | — | — |
| | Cinnamon | Psoriasis | 67 | 43.869 | 27.737 | 3.703 | 176.46 |
| | | Control | 120 | 37.244 | 25.730 | 5.064 | 147.88 |
| | | Diff (1 − 2) | — | 6.624 | 26.463 | — | — |
| | Clam | Psoriasis | 67 | 63.287 | 44.897 | 3.599 | 199.40 |
| | | Control | 120 | 46.602 | 35.142 | 9.651 | 207.57 |
| | | Diff (1 − 2) | — | 16.686 | 38.904 | — | — |
| | Codfish | Psoriasis | 67 | 23.816 | 47.824 | 1.763 | 400.00 |
| | | Control | 120 | 30.941 | 42.235 | 3.190 | 385.08 |
| | | Diff (1 − 2) | — | −7.125 | 44.310 | — | — |
| | Coffee | Psoriasis | 67 | 35.066 | 69.440 | 1.164 | 400.00 |
| | | Control | 120 | 20.736 | 20.293 | 2.522 | 111.30 |
| | | Diff (1 − 2) | — | 14.331 | 44.555 | — | — |
| | Cola_Nut | Psoriasis | 67 | 43.487 | 21.300 | 8.679 | 113.02 |
| | | Control | 120 | 34.448 | 16.528 | 9.778 | 93.693 |
| | | Diff (1 − 2) | — | 9.040 | 18.373 | — | — |
| | Corn | Psoriasis | 67 | 22.141 | 39.316 | 1.587 | 296.82 |
| | | Control | 120 | 12.279 | 23.585 | 1.151 | 222.95 |
| | | Diff (1 − 2) | — | 9.862 | 30.154 | — | — |
| | Cottage_Ch_{—} | Psoriasis | 67 | 148.673 | 153.331 | 1.719 | 400.00 |
| | | Control | 120 | 78.084 | 88.553 | 2.230 | 400.00 |
| | | Diff (1 − 2) | — | 70.589 | 115.894 | — | — |
| | Cow_Milk | Psoriasis | 67 | 143.436 | 146.344 | 1.058 | 400.00 |
| | | Control | 120 | 75.003 | 84.042 | 1.465 | 400.00 |
| | | Diff (1 − 2) | — | 68.434 | 110.380 | — | — |
| | Crab | Psoriasis | 67 | 37.438 | 41.118 | 1.161 | 195.05 |
| | | Control | 120 | 34.136 | 38.768 | 4.906 | 264.34 |
| | | Diff (1 − 2) | — | 3.302 | 39.623 | — | — |
| | Cucumber | Psoriasis | 67 | 17.544 | 17.069 | 0.952 | 71.952 |
| | | Control | 120 | 7.744 | 6.270 | 0.920 | 33.408 |
| | | Diff (1 − 2) | — | 9.800 | 11.368 | — | — |
| | Egg | Psoriasis | 67 | 38.702 | 57.835 | 1.164 | 294.76 |
| | | Control | 120 | 50.344 | 75.665 | 0.925 | 400.00 |
| | | Diff (1 − 2) | — | −11.643 | 69.828 | — | — |
| | Eggplant | Psoriasis | 67 | 12.335 | 15.461 | 0.846 | 67.624 |
| | | Control | 120 | 5.322 | 5.491 | 0.112 | 39.232 |
| | | Diff (1 − 2) | — | 7.014 | 10.231 | — | — |
| | Garlic | Psoriasis | 67 | 27.412 | 28.856 | 4.096 | 137.10 |
| | | Control | 120 | 15.507 | 14.140 | 3.034 | 88.882 |
| | | Diff (1 − 2) | — | 11.905 | 20.632 | — | — |
| | Goat_Milk | Psoriasis | 67 | 37.833 | 55.624 | 0.752 | 248.63 |
| | | Control | 120 | 15.413 | 17.918 | 0.553 | 101.25 |
| | | Diff (1 − 2) | — | 22.420 | 36.198 | — | — |
| | Grape | Psoriasis | 67 | 22.838 | 14.081 | 5.237 | 66.666 |
| | | Control | 120 | 20.624 | 7.921 | 6.592 | 57.274 |
| | | Diff (1 − 2) | — | 2.214 | 10.540 | — | — |
| | Grapefruit | Psoriasis | 67 | 8.925 | 14.134 | 0.100 | 75.630 |
| | | Control | 120 | 3.344 | 2.412 | 0.100 | 15.426 |
| | | Diff (1 − 2) | — | 5.581 | 8.661 | — | — |
| | Green_Pea | Psoriasis | 67 | 16.145 | 15.519 | 1.393 | 59.863 |
| | | Control | 120 | 12.264 | 16.995 | 0.100 | 106.01 |
| | | Diff (1 − 2) | — | 3.881 | 16.484 | — | — |
| | Green_Pepper | Psoriasis | 67 | 10.681 | 13.167 | 0.397 | 54.044 |
| | | Control | 120 | 4.275 | 3.376 | 0.100 | 19.874 |
| | | Diff (1 − 2) | — | 6.406 | 8.318 | — | — |
| | Halibut | Psoriasis | 67 | 11.673 | 7.576 | 1.858 | 39.672 |
| | | Control | 120 | 11.584 | 6.219 | 1.257 | 34.431 |
| | | Diff (1 − 2) | — | 0.089 | 6.735 | — | — |
| | Honey | Psoriasis | 67 | 15.694 | 10.513 | 1.879 | 50.951 |
| | | Control | 120 | 10.508 | 5.967 | 0.571 | 37.570 |
| | | Diff (1 − 2) | — | 5.186 | 7.895 | — | — |
| | Lemon | Psoriasis | 67 | 4.721 | 7.888 | 0.100 | 48.492 |
| | | Control | 120 | 2.433 | 1.778 | 0.100 | 11.844 |
| | | Diff (1 − 2) | — | 2.288 | 4.923 | — | — |
| | Lettuce | Psoriasis | 67 | 12.890 | 9.459 | 1.858 | 47.917 |
| | | Control | 120 | 14.631 | 14.739 | 3.452 | 96.804 |
| | | Diff (1 − 2) | — | −1.741 | 13.102 | — | — |
| | Lima_Bean | Psoriasis | 67 | 8.603 | 6.505 | 0.100 | 41.768 |
| | | Control | 120 | 8.046 | 9.019 | 0.971 | 68.661 |
| | | Diff (1 − 2) | — | 0.557 | 8.211 | — | — |
| | Lobster | Psoriasis | 67 | 17.441 | 14.390 | 1.164 | 79.720 |
| | | Control | 120 | 18.803 | 15.191 | 3.224 | 101.76 |
| | | Diff (1 − 2) | — | −1.362 | 14.910 | — | — |
| | Malt | Psoriasis | 67 | 30.210 | 17.452 | 3.903 | 74.672 |
| | | Control | 120 | 21.597 | 11.498 | 3.133 | 56.290 |
| | | Diff (1 − 2) | — | 8.613 | 13.918 | — | — |
| | Millet | Psoriasis | 67 | 4.312 | 2.666 | 0.931 | 15.943 |
| | | Control | 120 | 4.840 | 7.166 | 0.100 | 56.380 |
| | | Diff (1 − 2) | — | −0.529 | 5.964 | — | — |
| | Mushroom | Psoriasis | 67 | 13.841 | 13.351 | 0.661 | 64.842 |
| | | Control | 120 | 15.151 | 21.062 | 0.756 | 150.46 |
| | | Diff (1 − 2) | — | −1.310 | 18.680 | — | — |
| | Mustard | Psoriasis | 67 | 18.451 | 20.701 | 1.269 | 89.895 |
| | | Control | 120 | 10.473 | 7.851 | 1.004 | 48.101 |
| | | Diff (1 − 2) | — | 7.978 | 13.876 | — | — |
| | Oat | Psoriasis | 67 | 44.494 | 52.195 | 2.542 | 290.07 |
| | | Control | 120 | 18.633 | 21.889 | 2.160 | 143.48 |
| | | Diff (1 − 2) | — | 25.861 | 35.779 | — | — |
| | Olive | Psoriasis | 67 | 31.962 | 26.949 | 3.148 | 107.32 |
| | | Control | 120 | 22.137 | 15.571 | 5.503 | 100.38 |
| | | Diff (1 − 2) | — | 9.825 | 20.373 | — | — |
| | Onion | Psoriasis | 67 | 24.735 | 30.803 | 1.481 | 167.19 |
| | | Control | 120 | 12.459 | 14.850 | 2.072 | 94.943 |
| | | Diff (1 − 2) | — | 12.275 | 21.917 | — | — |
| | Orange | Psoriasis | 67 | 31.057 | 24.919 | 2.321 | 122.07 |
| | | Control | 120 | 19.878 | 20.985 | 2.158 | 137.98 |
| | | Diff (1 − 2) | — | 11.179 | 22.468 | — | — |
| | Oyster | Psoriasis | 67 | 83.210 | 100.148 | 7.678 | 400.00 |
| | | Control | 120 | 60.800 | 63.588 | 7.755 | 400.00 |
| | | Diff (1 − 2) | — | 22.409 | 78.607 | — | — |
| | Parsley | Psoriasis | 67 | 4.843 | 8.179 | 0.100 | 61.337 |
| | | Control | 120 | 8.940 | 20.778 | 0.100 | 143.39 |
| | | Diff (1 − 2) | — | −4.097 | 17.366 | — | — |
| | Peach | Psoriasis | 67 | 29.030 | 68.647 | 0.219 | 400.00 |
| | | Control | 120 | 6.617 | 6.996 | 0.100 | 35.954 |
| | | Diff (1 − 2) | — | 22.414 | 41.384 | — | — |
| | Peanut | Psoriasis | 67 | 6.394 | 5.648 | 0.698 | 32.385 |
| | | Control | 120 | 7.099 | 11.916 | 0.100 | 72.177 |
| | | Diff (1 − 2) | — | −0.705 | 10.134 | — | — |
| | Pineapple | Psoriasis | 67 | 51.151 | 78.331 | 1.879 | 400.00 |
| | | Control | 120 | 19.200 | 32.637 | 0.100 | 224.86 |
| | | Diff (1 − 2) | — | 31.951 | 53.611 | — | — |
| | Pinto_Bean | Psoriasis | 67 | 13.990 | 14.660 | 1.509 | 63.774 |
| | | Control | 120 | 10.179 | 8.220 | 3.076 | 78.334 |
| | | Diff (1 − 2) | — | 3.811 | 10.961 | — | — |
| | Pork | Psoriasis | 67 | 25.403 | 67.110 | 1.904 | 400.00 |
| | | Control | 120 | 16.887 | 32.923 | 2.848 | 352.54 |
| | | Diff (1 − 2) | — | 8.515 | 48.000 | — | — |
| | Potato | Psoriasis | 67 | 17.425 | 14.056 | 3.597 | 66.390 |
| | | Control | 120 | 13.287 | 4.968 | 4.321 | 30.493 |
| | | Diff (1 − 2) | — | 4.138 | 9.293 | — | — |
| | Rice | Psoriasis | 67 | 34.151 | 33.596 | 3.492 | 155.91 |
| | | Control | 120 | 24.295 | 18.422 | 2.701 | 119.70 |
| | | Diff (1 − 2) | — | 9.856 | 24.919 | — | — |
| | Rye | Psoriasis | 67 | 11.542 | 14.157 | 0.656 | 66.368 |
| | | Control | 120 | 5.514 | 3.891 | 0.100 | 30.398 |
| | | Diff (1 − 2) | — | 6.028 | 9.014 | — | — |
| | Safflower | Psoriasis | 67 | 10.880 | 7.524 | 1.322 | 44.283 |
| | | Control | 120 | 8.209 | 4.936 | 0.343 | 31.367 |
| | | Diff (1 − 2) | — | 2.671 | 5.989 | — | — |
| | Salmon | Psoriasis | 67 | 10.786 | 11.056 | 1.269 | 63.548 |
| | | Control | 120 | 10.261 | 8.222 | 1.573 | 55.715 |
| | | Diff (1 − 2) | — | 0.525 | 9.332 | — | — |
| | Sardine | Psoriasis | 67 | 44.786 | 18.205 | 7.544 | 81.687 |
| | | Control | 120 | 40.880 | 19.764 | 0.544 | 115.41 |
| | | Diff (1 − 2) | — | 3.906 | 19.222 | — | — |
| | Scallop | Psoriasis | 67 | 80.760 | 56.137 | 4.876 | 265.50 |
| | | Control | 120 | 75.524 | 36.235 | 1.284 | 182.33 |
| | | Diff (1 − 2) | — | 5.236 | 44.371 | — | — |
| | Sesame | Psoriasis | 67 | 59.270 | 81.189 | 2.010 | 400.00 |
| | | Control | 120 | 55.573 | 70.634 | 0.878 | 400.00 |
| | | Diff (1 − 2) | — | 3.697 | 74.571 | — | — |
| | Shrimp | Psoriasis | 67 | 29.808 | 29.715 | 1.904 | 136.42 |
| | | Control | 120 | 38.469 | 43.289 | 0.661 | 400.00 |
| | | Diff (1 − 2) | — | −8.661 | 38.992 | — | — |
| | Sole | Psoriasis | 67 | 5.644 | 3.051 | 0.529 | 18.266 |
| | | Control | 120 | 7.084 | 16.070 | 0.097 | 176.86 |
| | | Diff (1 − 2) | — | −1.440 | 13.017 | — | — |
| | Soybean | Psoriasis | 67 | 18.957 | 14.730 | 1.862 | 91.453 |
| | | Control | 120 | 19.618 | 20.367 | 0.206 | 150.95 |
| | | Diff (1 − 2) | — | −0.661 | 18.554 | — | — |
| | Spinach | Psoriasis | 67 | 30.882 | 30.214 | 3.715 | 113.82 |
| | | Control | 120 | 17.084 | 11.299 | 0.190 | 78.744 |
| | | Diff (1 − 2) | — | 13.798 | 20.194 | — | — |
| | Squashes | Psoriasis | 67 | 16.911 | 13.416 | 2.645 | 78.444 |
| | | Control | 120 | 14.525 | 12.798 | 0.212 | 82.645 |
| | | Diff (1 − 2) | — | 2.386 | 13.022 | — | — |
| | Strawberry | Psoriasis | 67 | 9.202 | 11.896 | 0.221 | 72.835 |
| | | Control | 120 | 6.108 | 11.226 | 0.158 | 117.33 |
| | | Diff (1 − 2) | — | 3.095 | 11.470 | — | — |
| | String_Bean | Psoriasis | 67 | 51.187 | 26.596 | 12.180 | 145.27 |
| | | Control | 120 | 46.296 | 26.174 | 0.613 | 147.79 |
| | | Diff (1 − 2) | — | 4.891 | 26.325 | — | — |
| | Sunflower_Sd | Psoriasis | 67 | 13.992 | 11.936 | 1.280 | 64.776 |
| | | Control | 120 | 10.659 | 7.874 | 0.125 | 55.601 |
| | | Diff (1 − 2) | — | 3.333 | 9.524 | — | — |
| | Sweet_Pot_{—} | Psoriasis | 67 | 17.346 | 20.812 | 1.718 | 105.66 |
| | | Control | 120 | 8.884 | 6.498 | 0.133 | 50.719 |
| | | Diff (1 − 2) | — | 8.462 | 13.479 | — | — |
| | Swiss_Ch_{—} | Psoriasis | 67 | 94.518 | 125.081 | 0.537 | 400.00 |
| | | Control | 120 | 35.610 | 45.054 | 0.249 | 227.39 |
| | | Diff (1 − 2) | — | 58.908 | 82.989 | — | — |
| | Tea | Psoriasis | 67 | 39.897 | 21.816 | 9.845 | 106.19 |
| | | Control | 120 | 29.006 | 11.822 | 0.292 | 67.899 |
| | | Diff (1 − 2) | — | 10.891 | 16.115 | — | — |
| | Tobacco | Psoriasis | 67 | 50.775 | 31.603 | 7.675 | 197.71 |
| | | Control | 120 | 37.107 | 24.996 | 0.255 | 185.36 |
| | | Diff (1 − 2) | — | 13.668 | 27.536 | — | — |
| | Tomato | Psoriasis | 67 | 25.375 | 47.435 | 1.658 | 266.03 |
| | | Control | 120 | 8.734 | 9.383 | 0.121 | 80.067 |
| | | Diff (1 − 2) | — | 16.641 | 29.315 | — | — |
| | Trout | Psoriasis | 67 | 14.406 | 12.290 | 2.561 | 70.436 |
| | | Control | 120 | 17.960 | 14.790 | 0.169 | 109.24 |
| | | Diff (1 − 2) | — | −3.553 | 13.950 | — | — |
| | Tuna | Psoriasis | 67 | 15.597 | 13.183 | 0.793 | 78.014 |
| | | Control | 120 | 17.583 | 13.172 | 0.189 | 93.539 |
| | | Diff (1 − 2) | — | −1.986 | 13.176 | — | — |
| | Turkey | Psoriasis | 67 | 14.921 | 14.977 | 2.539 | 121.32 |
| | | Control | 120 | 16.465 | 10.055 | 0.228 | 49.751 |
| | | Diff (1 − 2) | — | −1.544 | 12.044 | — | — |
| | Walnut_Blk | Psoriasis | 67 | 37.689 | 32.233 | 4.232 | 153.60 |
| | | Control | 120 | 27.829 | 17.399 | 0.157 | 112.07 |
| | | Diff (1 − 2) | — | 9.860 | 23.778 | — | — |
| | Wheat | Psoriasis | 67 | 49.819 | 82.936 | 2.328 | 393.32 |
| | | Control | 120 | 15.824 | 13.755 | 0.125 | 94.588 |
| | | Diff (1 − 2) | — | 33.995 | 50.750 | — | — |
| | Yeast_Baker | Psoriasis | 67 | 9.296 | 11.376 | 0.582 | 72.057 |
| | | Control | 120 | 6.922 | 7.362 | 0.074 | 47.574 |
| | | Diff (1 − 2) | — | 2.374 | 9.002 | — | — |
| | Yeast_Brewer | Psoriasis | 67 | 19.343 | 23.727 | 0.931 | 135.85 |
| | | Control | 120 | 14.452 | 17.389 | 0.101 | 100.26 |
| | | Diff (1 − 2) | — | 4.891 | 19.883 | — | — |
| | Yogurt | Psoriasis | 67 | 50.145 | 66.551 | 0.931 | 280.93 |
| | | Control | 120 | 22.386 | 23.180 | 0.321 | 136.19 |
| | | Diff (1 − 2) | — | 27.760 | 43.883 | — | — |

TABLE 4
Upper Quantiles of ELISA Signal Scores among Control Subjects as
Candidates for Test Cutpoints in Determining “Positive” or “Negative”
Top 59 Foods Ranked by Descending order of Discriminatory Ability
using Permutation Test

| | | | Cutpoint | |
|---|---|---|---|---|
| Food | | | 90th | 95th |
| Ranking | Food | Sex | percentile | percentile |
| 1 | Peach | FEMALE | 18.366 | 23.671 |
| | | MALE | 15.233 | 23.190 |
| 2 | Cucumber | FEMALE | 16.978 | 23.451 |
| | | MALE | 16.129 | 21.988 |
| 3 | Tea | FEMALE | 52.232 | 59.023 |
| | | MALE | 44.521 | 49.474 |
| 4 | Tomato | FEMALE | 17.176 | 24.934 |
| | | MALE | 17.889 | 23.383 |
| 5 | Broccoli | FEMALE | 11.120 | 13.707 |
| | | MALE | 10.767 | 15.005 |
| 6 | Cauliflower | FEMALE | 8.101 | 10.487 |
| | | MALE | 10.181 | 13.715 |
| 7 | Almond | FEMALE | 7.119 | 9.242 |
| | | MALE | 9.912 | 12.749 |
| 8 | Green_Pepper | FEMALE | 8.310 | 9.809 |
| | | MALE | 8.146 | 11.168 |
| 9 | Grapefruit | FEMALE | 6.395 | 7.795 |
| | | MALE | 6.506 | 8.108 |
| 10 | Tobacco | FEMALE | 68.234 | 83.037 |
| | | MALE | 67.010 | 79.772 |
| 11 | Eggplant | FEMALE | 9.830 | 16.881 |
| | | MALE | 11.432 | 14.794 |
| 12 | Rye | FEMALE | 9.337 | 12.113 |
| | | MALE | 9.269 | 12.298 |
| 13 | Oat | FEMALE | 46.854 | 68.118 |
| | | MALE | 41.582 | 57.396 |
| 14 | Cantaloupe | FEMALE | 11.409 | 13.800 |
| | | MALE | 11.573 | 13.558 |
| 15 | Cabbage | FEMALE | 12.730 | 17.087 |
| | | MALE | 11.422 | 17.567 |
| 16 | Cane_Sugar | FEMALE | 40.065 | 53.675 |
| | | MALE | 38.137 | 49.436 |
| 17 | Sweet_Pot_{—} | FEMALE | 14.044 | 17.261 |
| | | MALE | 14.327 | 20.310 |
| 18 | Pineapple | FEMALE | 47.138 | 84.380 |
| | | MALE | 50.766 | 87.306 |
| 19 | Avocado | FEMALE | 4.508 | 6.111 |
| | | MALE | 4.376 | 5.474 |
| 20 | Orange | FEMALE | 47.023 | 72.520 |
| | | MALE | 44.043 | 61.717 |
| 21 | Spinach | FEMALE | 30.407 | 39.841 |
| | | MALE | 29.469 | 37.447 |
| 22 | Honey | FEMALE | 17.390 | 22.188 |
| | | MALE | 17.629 | 22.161 |
| 23 | Swiss_Ch_{—} | FEMALE | 125.53 | 246.90 |
| | | MALE | 87.170 | 143.18 |
| 24 | Malt | FEMALE | 42.458 | 48.828 |
| | | MALE | 37.608 | 43.367 |
| 25 | Mustard | FEMALE | 16.576 | 18.807 |
| | | MALE | 19.286 | 26.442 |
| 26 | Wheat | FEMALE | 34.767 | 58.125 |
| | | MALE | 30.214 | 40.845 |
| 27 | Apple | FEMALE | 8.916 | 11.286 |
| | | MALE | 8.549 | 13.177 |
| 28 | Chocolate | FEMALE | 32.479 | 37.492 |
| | | MALE | 27.159 | 33.055 |
| 29 | Yogurt | FEMALE | 52.355 | 69.899 |
| | | MALE | 46.826 | 66.534 |
| 30 | Goat_Milk | FEMALE | 32.938 | 66.032 |
| | | MALE | 38.223 | 53.932 |
| 31 | Cola_Nut | FEMALE | 60.409 | 64.983 |
| | | MALE | 56.175 | 63.576 |
| 32 | Clam | FEMALE | 75.147 | 93.874 |
| | | MALE | 88.303 | 112.57 |
| 33 | Cheddar_Ch_{—} | FEMALE | 110.14 | 162.22 |
| | | MALE | 56.509 | 80.656 |
| 34 | Olive | FEMALE | 46.417 | 60.040 |
| | | MALE | 43.078 | 50.905 |
| 35 | Yeast_Brewer | FEMALE | 25.085 | 32.400 |
| | | MALE | 31.874 | 48.190 |
| 36 | Butter | FEMALE | 55.376 | 71.051 |
| | | MALE | 53.978 | 66.916 |
| 37 | Celery | FEMALE | 22.392 | 29.399 |
| | | MALE | 18.785 | 30.373 |
| 38 | Onion | FEMALE | 28.218 | 42.358 |
| | | MALE | 26.807 | 42.455 |
| 39 | Garlic | FEMALE | 23.997 | 39.823 |
| | | MALE | 27.773 | 43.316 |
| 40 | Walnut_Blk | FEMALE | 46.650 | 66.072 |
| | | MALE | 46.713 | 60.996 |
| 41 | Cottage_Ch_{—} | FEMALE | 252.56 | 376.95 |
| | | MALE | 194.81 | 271.45 |
| 42 | Yeast_Baker | FEMALE | 10.825 | 15.561 |
| | | MALE | 12.748 | 18.794 |
| 43 | Cow_Milk | FEMALE | 236.99 | 355.64 |
| | | MALE | 192.77 | 255.70 |
| 44 | Corn | FEMALE | 18.329 | 33.786 |
| | | MALE | 22.657 | 35.960 |
| 45 | Amer_Cheese | FEMALE | 86.030 | 146.07 |
| | | MALE | 47.540 | 73.790 |
| 46 | Strawberry | FEMALE | 9.258 | 14.782 |
| | | MALE | 10.629 | 15.268 |
| 47 | Buck_Wheat | FEMALE | 13.545 | 17.598 |
| | | MALE | 14.037 | 17.446 |
| 48 | Lemon | FEMALE | 4.445 | 6.001 |
| | | MALE | 4.209 | 5.714 |
| 49 | Green_Pea | FEMALE | 26.822 | 49.810 |
| | | MALE | 24.182 | 51.333 |
| 50 | Trout | FEMALE | 35.184 | 49.914 |
| | | MALE | 29.051 | 37.187 |
| 51 | Barley | FEMALE | 45.693 | 57.123 |
| | | MALE | 39.460 | 55.067 |
| 52 | Potato | FEMALE | 19.569 | 25.620 |
| | | MALE | 20.158 | 22.292 |
| 53 | Beef | FEMALE | 14.699 | 20.083 |
| | | MALE | 11.939 | 19.689 |
| 54 | Rice | FEMALE | 45.656 | 67.990 |
| | | MALE | 46.617 | 62.770 |
| 55 | Sunflower_Sd | FEMALE | 20.574 | 30.655 |
| | | MALE | 17.384 | 24.496 |
| 56 | Chili_Pepper | FEMALE | 18.264 | 29.015 |
| | | MALE | 20.710 | 35.019 |
| 57 | Banana | FEMALE | 12.516 | 17.556 |
| | | MALE | 13.351 | 24.350 |
| 58 | String_Bean | FEMALE | 75.632 | 100.65 |
| | | MALE | 83.264 | 103.46 |
| 59 | Safflower | FEMALE | 16.360 | 23.394 |
| | | MALE | 14.018 | 16.975 |

TABLE 5A

| | |
|---|---|
| | # of Positive Results Based |
| Sample ID | on 90th Percentile |

PSORIASIS POPULATION

| | |
|---|---|
| KH16-12764 | 18 |
| KH16-13276 | 14 |
| KH16-13571 | 10 |
| KH16-13573 | 46 |
| KH16-13877 | 49 |
| KH16-14181 | 2 |
| KH16-14182 | 1 |
| KH16-14184 | 25 |
| KH16-14185 | 4 |
| KH16-14186 | 50 |
| KH16-14582 | 59 |
| BRH1226007 | 37 |
| BRH1226011 | 26 |
| BRH1226013 | 29 |
| BRH1226015 | 38 |
| BRH1226016 | 30 |
| BRH1226020 | 0 |
| BRH1226021 | 2 |
| BRH1226022 | 18 |
| BRH1226024 | 8 |
| BRH1217480 | 7 |
| BRH1217481 | 11 |
| BRH1217483 | 47 |
| BRH1217485 | 1 |
| BRH1217486 | 47 |
| BRH1217489 | 5 |
| BRH1217490 | 2 |
| BRH1217491 | 46 |
| BRH1217492 | 4 |
| BRH1217494 | 4 |
| BRH1217497 | 21 |
| BRH1217498 | 39 |
| BRH1217501 | 11 |
| BRH1217502 | 2 |
| BRH1217503 | 32 |
| BRH1217504 | 0 |
| BRH1217507 | 49 |
| KH15-16815 | 0 |
| KH15-17685 | 1 |
| KH15-18901 | 6 |
| KH16-01608 | 20 |
| KH16-04038 | 10 |
| KH16-04039 | 12 |
| KH16-04313 | 6 |
| KH16-04885 | 23 |
| KH16-05027 | 1 |
| KH16-05483 | 4 |
| KH16-06929 | 8 |
| KH16-06932 | 5 |
| KH16-08306 | 10 |
| KH16-08307 | 3 |
| KH16-08560 | 2 |
| BRH1214586 | 2 |
| BRH1214587 | 0 |
| BRH1214588 | 0 |
| BRH1214590 | 2 |
| BRH1214593 | 40 |
| BRH1214594 | 37 |
| BRH1214596 | 2 |
| BRH1214597 | 4 |
| BRH1214599 | 6 |
| BRH1214600 | 12 |
| BRH1214604 | 6 |
| BRH1214606 | 24 |
| BRH1214607 | 4 |
| BRH1214608 | 34 |
| BRH1214609 | 2 |
| KH-1898 | 12 |
| KH-1899 | 6 |
| KH16-10295 | 1 |
| KH16-12582 | 6 |
| KH16-12584 | 19 |
| KH16-12763 | 2 |
| KH16-12765 | 10 |
| KH16-13277 | 9 |
| KH16-13570 | 33 |
| KH16-13876 | 0 |
| KH16-14183 | 8 |
| KH16-15441 | 47 |
| KH16-15641 | 22 |
| KH16-16345 | 1 |
| BRH1226008 | 9 |
| BRH1226009 | 1 |
| BRH1226010 | 32 |
| BRH1226012 | 9 |
| BRH1226014 | 1 |
| BRH1226017 | 5 |
| BRH1226018 | 10 |
| BRH1226019 | 11 |
| BRH1226023 | 18 |
| BRH1217482 | 22 |
| BRH1217484 | 10 |
| BRH1217487 | 9 |
| BRH1217488 | 7 |
| BRH1217493 | 10 |
| BRH1217495 | 7 |
| BRH1217496 | 12 |
| BRH1217499 | 3 |
| BRH1217500 | 58 |
| BRH1217505 | 13 |
| BRH1217506 | 2 |
| KH15-16733 | 44 |
| KH15-16812 | 0 |
| KH15-17088 | 6 |
| KH15-17385 | 31 |
| KH15-18902 | 50 |
| KH16-00804 | 6 |
| KH16-00805 | 2 |
| KH16-01745 | 6 |
| KH16-01748 | 9 |
| KH16-02280 | 0 |
| KH16-02752 | 13 |
| KH16-02753 | 3 |
| KH16-02872 | 2 |
| KH16-02896 | 0 |
| KH16-03138 | 6 |
| KH16-03898 | 3 |
| KH16-04886 | 2 |
| KH16-05028 | 2 |
| KH16-05627 | 40 |
| KH16-07760 | 2 |
| BRH1214589 | 5 |
| BRH1214591 | 1 |
| BRH1214592 | 0 |
| BRH1214595 | 0 |
| BRH1214598 | 11 |
| BRH1214601 | 15 |
| BRH1214602 | 2 |
| BRH1214603 | 8 |
| BRH1214605 | 7 |
| BRH1214610 | 11 |
| BRH1214611 | 7 |
| BRH1214612 | 9 |
| No of Observations | 133 |
| Average Number | 13.6 |
| Median Number | 8 |
| # of Patients w/0 Pos Results | 11 |
| % Subjects w/0 pos results | 8.3 |

NON-PSORIASIS POPULATION

| | |
|---|---|
| BRH1165675 | 14 |
| BRH1165676 | 8 |
| BRH1165677 | 0 |
| BRH1165678 | 2 |
| BRH1165679 | 8 |
| BRH1165680 | 4 |
| BRH1165681 | 1 |
| BRH1165682 | 22 |
| BRH1165683 | 8 |
| BRH1165684 | 6 |
| BRH1165698 | 2 |
| BRH1165700 | 0 |
| BRH1165701 | 6 |
| BRH1165703 | 9 |
| BRH1165704 | 31 |
| BRH1165705 | 2 |
| BRH1165706 | 1 |
| BRH1165707 | 1 |
| BRH1165709 | 6 |
| BRH1165710 | 12 |
| BRH1165747 | 1 |
| BRH1165748 | 10 |
| BRH1165749 | 6 |
| BRH1165750 | 1 |
| BRH1165751 | 4 |
| BRH1165752 | 1 |
| BRH1165772 | 22 |
| BRH1165773 | 6 |
| BRH1165774 | 1 |
| BRH1165775 | 2 |
| BRH1165777 | 6 |
| BRH1209177 | 0 |
| BRH1209182 | 1 |
| BRH1209183 | 1 |
| BRH1209184 | 1 |
| BRH1209187 | 7 |
| BRH1209197 | 20 |
| BRH1209198 | 0 |
| BRH1209199 | 5 |
| BRH1209200 | 10 |
| BRH1209201 | 5 |
| BRH1209212 | 3 |
| BRH1209213 | 3 |
| BRH1209214 | 0 |
| BRH1209215 | 2 |
| BRH1209216 | 9 |
| BRH1209217 | 0 |
| BRH1209218 | 0 |
| BRH1209219 | 0 |
| BRH1209220 | 8 |
| BRH1209221 | 0 |
| BRH1209238 | 1 |
| BRH1209239 | 7 |
| BRH1209240 | 0 |
| BRH1209241 | 9 |
| BRH1209243 | 1 |
| BRH1209256 | 15 |
| BRH1209257 | 0 |
| BRH1209258 | 5 |
| BRH1209259 | 10 |
| BRH1165685 | 6 |
| BRH1165688 | 0 |
| BRH1165690 | 2 |
| BRH1165691 | 2 |
| BRH1165692 | 44 |
| BRH1165694 | 2 |
| BRH1165695 | 4 |
| BRH1165711 | 6 |
| BRH1165712 | 2 |
| BRH1165713 | 9 |
| BRH1165714 | 11 |
| BRH1165715 | 11 |
| BRH1165716 | 28 |
| BRH1165717 | 4 |
| BRH1165718 | 4 |
| BRH1165719 | 2 |
| BRH1165722 | 1 |
| BRH1165723 | 1 |
| BRH116S724 | 1 |
| BRH1165725 | 5 |
| BRH1165726 | 7 |
| BRH1165727 | 2 |
| BRH1165729 | 2 |
| BRH1165730 | 0 |
| BRH1165731 | 2 |
| BRH1165733 | 6 |
| BRH1165734 | 12 |
| BRH1165736 | 0 |
| BRH1165739 | 6 |
| BRH1165740 | 13 |
| BRH1165742 | 1 |
| BRH1165746 | 12 |
| BRH1165753 | 6 |
| BRH1165754 | 10 |
| BRH1165755 | 8 |
| BRH1165756 | 3 |
| BRH1165758 | 0 |
| BRH1165759 | 0 |
| BRH1165761 | 1 |
| BRH1165762 | 13 |
| BRH1165767 | 2 |
| BRH1165768 | 2 |
| BRH1165770 | 1 |
| BRH1165771 | 4 |
| BRH1209188 | 1 |
| BRH1209189 | 1 |
| BRH1209190 | 24 |
| BRH1209191 | 7 |
| BRH1209193 | 10 |
| BRH1209194 | 2 |
| BRH1209195 | 5 |
| BRH1209196 | 3 |
| BRH1209202 | 1 |
| BRH1209203 | 0 |
| BRH1209205 | 6 |
| BRH1209206 | 2 |
| BRH1209207 | 4 |
| BRH1209208 | 21 |
| BRH1209209 | 26 |
| BRH1209210 | 1 |
| BRH1165779 | 23 |
| BRH1165780 | 1 |
| BRH1165781 | 1 |
| BRH1165784 | 1 |
| BRH1165785 | 30 |
| BRH1165805 | 5 |
| BRH1165806 | 11 |
| BRH1165807 | 6 |
| BRH1165811 | 3 |
| BRH1165812 | 1 |
| BRH1165821 | 1 |
| BRH1165822 | 0 |
| BRH1165823 | 4 |
| BRH1165824 | 28 |
| BRH1165825 | 5 |
| BRH1165846 | 18 |
| BRH1165847 | 26 |
| BRH1165848 | 28 |
| BRH1165850 | 2 |
| BRH1165851 | 8 |
| BRH1165852 | 8 |
| BRH1165853 | 12 |
| BRH1165856 | 2 |
| BRH1165858 | 7 |
| BRH1165859 | 1 |
| BRH1165860 | 3 |
| BRH1165861 | 3 |
| BRH1165862 | 12 |
| BRH1165864 | 0 |
| BRH1165866 | 23 |
| BRH1209262 | 9 |
| BRH-1209348 | 6 |
| BRH1209265 | 16 |
| BRH1209266 | 12 |
| BRH1209267 | 1 |
| BRH1209272 | 8 |
| BRH1209273 | 2 |
| BRH1209275 | 3 |
| BRH1209276 | 2 |
| BRH1209278 | 2 |
| BRH1209291 | 0 |
| BRH1209293 | 3 |
| BRH1209294 | 1 |
| BRH1209295 | 16 |
| BRH1209296 | 5 |
| BRH1209297 | 2 |
| BRH1209304 | 5 |
| BRH1209305 | 1 |
| BRH1209306 | 1 |
| BRH1209307 | 0 |
| BRH1209308 | 1 |
| BRH1209318 | 9 |
| BRH1209319 | 15 |
| BRH1209321 | 0 |
| BRH1209322 | 6 |
| BRH1209323 | 5 |
| BRH1209344 | 1 |
| BRH1209345 | 20 |
| BRH1209346 | 8 |
| BRH1209347 | 0 |
| BRH1165791 | 2 |
| BRH1165794 | 0 |
| BRH1165797 | 3 |
| BRH1165798 | 2 |
| BRH1165799 | 5 |
| BRH1165801 | 26 |
| BRH1165802 | 0 |
| BRH1165803 | 0 |
| BRH1165813 | 0 |
| BRH1165814 | 2 |
| BRH1165815 | 4 |
| BRH1165817 | 5 |
| BRH1165829 | 0 |
| BRH1165832 | 18 |
| BRH1165834 | 0 |
| BRH1165837 | 3 |
| BRH1165843 | 11 |
| BRH1209269 | 1 |
| BRH1209280 | 3 |
| BRH1209283 | 1 |
| BRH1209284 | 7 |
| BRH1209287 | 4 |
| BRH1209289 | 9 |
| BRH1209298 | 0 |
| BRH1209300 | 1 |
| BRH1209302 | 33 |
| BRH1209316 | 3 |
| BRH1209325 | 3 |
| BRH1209326 | 3 |
| BRH1209327 | 3 |
| BRH1209330 | 2 |
| BRH1209332 | 0 |
| BRH1209337 | 1 |
| BRH1209340 | 0 |
| BRH1209341 | 1 |
| BRH1244998 | 5 |
| BRH1244999 | 3 |
| BRH1245000 | 9 |
| BRH1245001 | 1 |
| BRH1245002 | 4 |
| BRH1245004 | 1 |
| BRH1245007 | 1 |
| BRH1245008 | 4 |
| BRH1245010 | 22 |
| BRH1245011 | 8 |
| BRH1245012 | 1 |
| BRH1245013 | 6 |
| BRH1245014 | 0 |
| BRH1245015 | 0 |
| BRH1245016 | 8 |
| BRH1245018 | 0 |
| BRH1245019 | 2 |
| BRH1245022 | 13 |
| BRH1245023 | 2 |
| BRH1245024 | 2 |
| BRH1244993 | 1 |
| BRH1244994 | 0 |
| BRH1244995 | 2 |
| BRH1244996 | 6 |
| BRH1244997 | 0 |
| No of Observations | 240 |
| Average Number | 5.8 |
| Median Number | 3 |
| # of Patients w/0 Pos Results | 37 |
| % Subjects w/0 pos results | 15.4 |

TABLE 5B

| | |
|---|---|
| | # of Positive Results Based |
| Sample ID | on 95th Percentile |

PSORIASIS POPULATION

| | |
|---|---|
| KH16-12764 | 12 |
| KH16-13276 | 11 |
| KH16-13571 | 9 |
| KH16-13573 | 36 |
| KH16-13877 | 38 |
| KH16-14181 | 0 |
| KH16-14182 | 1 |
| KH16-14184 | 15 |
| KH16-14185 | 1 |
| KH16-14186 | 40 |
| KH16-14582 | 54 |
| BRH1226007 | 15 |
| BRH1226011 | 24 |
| BRH1226013 | 20 |
| BRH1226015 | 30 |
| BRH1226016 | 23 |
| BRH1226020 | 0 |
| BRH1226021 | 1 |
| BRH1226022 | 10 |
| BRK1226024 | 6 |
| BRH1217480 | 3 |
| BRH1217481 | 4 |
| BRH1217483 | 40 |
| BRH1217485 | 0 |
| BRH1217486 | 36 |
| BRK1217489 | 3 |
| BRH1217490 | 2 |
| BRH1217491 | 37 |
| BRH1217492 | 2 |
| BRH1217494 | 2 |
| BRH1217497 | 13 |
| BRH1217498 | 30 |
| BRH1217501 | 9 |
| BRH1217502 | 0 |
| BRH1217503 | 27 |
| BRH1217504 | 0 |
| BRH1217507 | 38 |
| KH15-16815 | 0 |
| KH15-17685 | 0 |
| KH15-18901 | 2 |
| KH16-01608 | 16 |
| KH16-04038 | 8 |
| KH16-04039 | 8 |
| KH16-04313 | 3 |
| KH16-04885 | 18 |
| KH16-05027 | 0 |
| KH16-05483 | 1 |
| KH16-06929 | 3 |
| KH16-06932 | 2 |
| KH16-08306 | 8 |
| KH16-08307 | 2 |
| KH16-08560 | 0 |
| BRH1214586 | 1 |
| BRH1214587 | 0 |
| BRH1214588 | 0 |
| BRH1214590 | 1 |
| BRH1214593 | 36 |
| BRH1214594 | 29 |
| BRH1214596 | 2 |
| BRH1214597 | 1 |
| BRH1214599 | 4 |
| BRH1214600 | 7 |
| BRH1214604 | 4 |
| BRH1214606 | 11 |
| BRH1214607 | 4 |
| BRH1214608 | 20 |
| BRH1214609 | 1 |
| KH-1898 | 4 |
| KH-1899 | 4 |
| KH16-10295 | 1 |
| KH16-12582 | 3 |
| KH16-12584 | 11 |
| KH16-12763 | 1 |
| KH16-12765 | 7 |
| KH16-13277 | 4 |
| KH16-13570 | 14 |
| KH16-13876 | 0 |
| KH16-14183 | 2 |
| KH16-15441 | 46 |
| KH16-15641 | 18 |
| KH16-16345 | 1 |
| BRH1226008 | 7 |
| BRH1226009 | 0 |
| BRH1226010 | 15 |
| BRH1226012 | 1 |
| BRH1226014 | 0 |
| BRH1226017 | 2 |
| BRH1226018 | 6 |
| BRH1226019 | 7 |
| BRH1226023 | 8 |
| BRH1217482 | 18 |
| BRH1217484 | 7 |
| BRH1217487 | 8 |
| BRH1217488 | 1 |
| BRH1217493 | 7 |
| BRH1217495 | 3 |
| BRH1217496 | 3 |
| BRH1217499 | 2 |
| BRH1217500 | 53 |
| BRH1217505 | 3 |
| BRH1217506 | 1 |
| KH15-16733 | 40 |
| KH15-16812 | 0 |
| KH15-17088 | 2 |
| KH15-17385 | 26 |
| KH15-18902 | 44 |
| KH16-00804 | 6 |
| KH16-00805 | 2 |
| KH16-01745 | 4 |
| KH16-01748 | 4 |
| KH16-02280 | 0 |
| KH16-02752 | 7 |
| KH16-02753 | 1 |
| KH16-02872 | 1 |
| KH16-02896 | 0 |
| KH16-03138 | 4 |
| KH16-03898 | 1 |
| KH16-04886 | 1 |
| KH16-05028 | 0 |
| KH16-05627 | 39 |
| KH16-07760 | 2 |
| BRH1214589 | 3 |
| BRH1214591 | 0 |
| BRH1214592 | 0 |
| BRH1214595 | 0 |
| BRH1214598 | 8 |
| BRH1214601 | 12 |
| BRH1214602 | 0 |
| BRH1214603 | 2 |
| BRH1214605 | 5 |
| BRH1214610 | 3 |
| BRH1214611 | 3 |
| BRH1214612 | 4 |
| No of Observations | 133 |
| Average Number | 9.6 |
| Median Number | 4 |
| # of Patients w/0 Pos Results | 22 |
| % Subjects w/0 pos results | 16.5 |

NON-PSORIASIS POPULATION

| | |
|---|---|
| BRH1165675 | 7 |
| BRH1165676 | 3 |
| BRK1165677 | 0 |
| BRH1165678 | 1 |
| BRH1165679 | 3 |
| BRH1165680 | 1 |
| BRH1165681 | 0 |
| BRH1165682 | 11 |
| BRH1165683 | 4 |
| BRH1165684 | 0 |
| BRH1165698 | 0 |
| BRH1165700 | 0 |
| BRH1165701 | 3 |
| BRH1165703 | 8 |
| BRH1165704 | 17 |
| BRH1165705 | 2 |
| BRH1165706 | 1 |
| BRH1165707 | 1 |
| BRH1165709 | 4 |
| BRH1165710 | 8 |
| BRH1165747 | 0 |
| BRH1165748 | 5 |
| BRH1165749 | 6 |
| BRH1165750 | 1 |
| BRH1165751 | 2 |
| BRH1165752 | 0 |
| BRH1165772 | 11 |
| BRH1165773 | 3 |
| BRH1165774 | 0 |
| BRH1165775 | 1 |
| BRH1165777 | 6 |
| BRH1209177 | 0 |
| BRH1209182 | 1 |
| BRH1209183 | 0 |
| BRH1209184 | 0 |
| BRH1209187 | 1 |
| BRH1209197 | 6 |
| BRH1209198 | 0 |
| BRH1209199 | 2 |
| BRH1209200 | 4 |
| BRH1209201 | 4 |
| BRH1209212 | 1 |
| BRH1209213 | 3 |
| BRH1209214 | 0 |
| BRH1209215 | 0 |
| BRH1209216 | 6 |
| BRH1209217 | 0 |
| BRH1209218 | 0 |
| BRH1209219 | 0 |
| BRH1209220 | 5 |
| BRH1209221 | 0 |
| BRH1209238 | 1 |
| BRH1209239 | 2 |
| BRH1209240 | 0 |
| BRH1209241 | 4 |
| BRH1209243 | 0 |
| BRH1209256 | 5 |
| BRH1209257 | 0 |
| BRH1209258 | 1 |
| BRH1209259 | 5 |
| BRH1165685 | 4 |
| BRH1165688 | 0 |
| BRH1165690 | 1 |
| BRH1165691 | 2 |
| BRH1165692 | 23 |
| BRH1165694 | 2 |
| BRH1165695 | 1 |
| BRH1165711 | 3 |
| BRH1165712 | 1 |
| BRH1165713 | 6 |
| BRH1165714 | 4 |
| BRH1165715 | 7 |
| BRH1165716 | 12 |
| BRH1165717 | 1 |
| BRH1165718 | 2 |
| BRH1165719 | 1 |
| BRH1165722 | 1 |
| BRH1165723 | 0 |
| BRH1165724 | 0 |
| BRH1165725 | 2 |
| BRH1165726 | 2 |
| BRH1165727 | 1 |
| BRH1165729 | 1 |
| BRH1165730 | 0 |
| BRH1165731 | 0 |
| BRH1165733 | 1 |
| BRH1165734 | 3 |
| BRH1165736 | 0 |
| BRH1165739 | 4 |
| BRH1165740 | 6 |
| BRH1165742 | 0 |
| BRH1165746 | 8 |
| BRH1165753 | 2 |
| BRH1165754 | 1 |
| BRH1165755 | 4 |
| BRH1165756 | 2 |
| BRH1165758 | 0 |
| BRH1165759 | 0 |
| BRH1165761 | 0 |
| BRH1165762 | 5 |
| BRH1165767 | 0 |
| BRH1165768 | 0 |
| BRH1165770 | 1 |
| BRH1165771 | 2 |
| BRH1209188 | 0 |
| BRH1209189 | 1 |
| BRH1209190 | 12 |
| BRH1209191 | 5 |
| BRH1209193 | 8 |
| BRH1209194 | 2 |
| BRH1209195 | 4 |
| BRH1209196 | 0 |
| BRH1209202 | 1 |
| BRH1209203 | 0 |
| BRH1209205 | 4 |
| BRH1209206 | 2 |
| BRH1209207 | 0 |
| BRH1209208 | 11 |
| BRH1209209 | 16 |
| BRH1209210 | 0 |
| BRH1165779 | 9 |
| BRH1165780 | 0 |
| BRH1165781 | 1 |
| BRH1165784 | 0 |
| BRH1165785 | 26 |
| BRH1165805 | 4 |
| BRH1165806 | 6 |
| BRH1165807 | 5 |
| BRH1165811 | 0 |
| BRH1165812 | 0 |
| BRH1165821 | 0 |
| BRH1165822 | 0 |
| BRH1165823 | 1 |
| BRH1165824 | 16 |
| BRH1165825 | 1 |
| BRH1165846 | 9 |
| BRH1165847 | 16 |
| BRH1165848 | 17 |
| BRH1165850 | 1 |
| BRH1165851 | 0 |
| BRH1165852 | 7 |
| BRH1165853 | 9 |
| BRH1165856 | 1 |
| BRH1165858 | 2 |
| BRH1165859 | 0 |
| BRH1165860 | 2 |
| BRH1165861 | 3 |
| BRH1165862 | 6 |
| BRH1165864 | 0 |
| BRH1165866 | 13 |
| BRH1209262 | 7 |
| BRH-1209348 | 3 |
| BRH1209265 | 14 |
| BRH1209266 | 11 |
| BRH1209267 | 0 |
| BRH1209272 | 4 |
| BRH1209273 | 2 |
| BRH1209275 | 0 |
| BRH1209276 | 0 |
| BRH1209278 | 2 |
| BRH1209291 | 0 |
| BRH1209293 | 0 |
| BRH1209294 | 0 |
| BRH1209295 | 10 |
| BRH1209296 | 3 |
| BRH1209297 | 0 |
| BRH1209304 | 1 |
| BRH1209305 | 0 |
| BRH1209306 | 1 |
| BRH1209307 | 0 |
| BRH1209308 | 0 |
| BRH1209318 | 4 |
| BRH1209319 | 3 |
| BRH1209321 | 0 |
| BRH1209322 | 2 |
| BRH1209323 | 2 |
| BRH1209344 | 1 |
| BRH1209345 | 11 |
| BRH1209346 | 2 |
| BRH1209347 | 0 |
| BRH1165791 | 0 |
| BRH1165794 | 0 |
| BRH1165797 | 2 |
| BRH1165798 | 0 |
| BRH1165799 | 2 |
| BRH1165801 | 13 |
| BRH1165802 | 0 |
| BRH1165803 | 0 |
| BRH1165813 | 0 |
| BRH1165814 | 0 |
| BRH1165815 | 2 |
| BRH1165817 | 2 |
| BRH1165829 | 0 |
| BRH1165832 | 10 |
| BRH1165834 | 0 |
| BRH1165837 | 2 |
| BRH1165843 | 9 |
| BRH1209269 | 1 |
| BRH1209280 | 2 |
| BRH1209283 | 0 |
| BRH1209284 | 2 |
| BRH1209287 | 2 |
| BRH1209289 | 5 |
| BRH1209298 | 0 |
| BRH1209300 | 1 |
| BRH1209302 | 16 |
| BRH1209316 | 3 |
| BRH1209325 | 3 |
| BRH1209326 | 1 |
| BRH1209327 | 1 |
| BRH1209330 | 0 |
| BRH1209332 | 0 |
| BRH1209337 | 1 |
| BRH1209340 | 0 |
| BRH1209341 | 0 |
| BRH1244998 | 2 |
| BRH1244999 | 2 |
| BRH1245000 | 5 |
| BRH1245001 | 0 |
| BRH1245002 | 0 |
| BRH1245004 | 0 |
| BRH1245007 | 1 |
| BRH1245008 | 1 |
| BRH1245010 | 10 |
| BRH1245011 | 4 |
| BRH1245012 | 1 |
| BRH1245013 | 3 |
| BRH1245014 | 0 |
| BRH1245015 | 0 |
| BRH1245016 | 5 |
| BRH1245018 | 0 |
| BRH1245019 | 1 |
| BRH1245022 | 4 |
| BRH1245023 | 1 |
| BRH1245024 | 1 |
| BRH1244993 | 0 |
| BRH1244994 | 0 |
| BRH1244995 | 0 |
| BRH1244996 | 2 |
| BRH1244997 | 0 |
| No of Observations | 240 |
| Average Number | 2.9 |
| Median Number | 1 |
| # of Patients w/0 Pos Results | 84 |
| % Subjects w/0 pos results | 35.0 |

TABLE 6A

| Variable | | Psoriasis_90th_precentile | |
|---|---|---|---|
| | | Psoriasis 90th precentile | |
| Sample size | | 133 | |
| Lowest value | | 0.0000 | |
| Highest value | | 59.0000 | |
| Arithmetic mean | | 13.5940 | |
| 95% CI for the mean | | 10.9822 to 16.2058 | |
| Median | | 8.0000 | |
| 95% CI for the median | | 6.0000 to 10.0000 | |
| Variance | | 231.8642 | |
| Standard deviation | | 15.2271 | |
| Relative standard deviation | | 1.1201 | (112.01%) |
| Standard error of the mean | | 1.3204 | |
| Coefficient of Skewness | | 1.3613 | (P < 0.0001) |
| Coefficient of Kurtosis | | 0.7443 | (P = 0.1088) |
| D'Agostino-Pearson test | | reject Normality | (P < 0.0001) |
| for Normal distribution | | | |
| Percentiles | | 95% Confidence interval | |
| 2.5 | 0.0000 | | |
| 5 | 0.0000 | 0.0000 to 1.0000 | |
| 10 | 1.0000 | 0.0000 to 1.9227 | |
| 25 | 2.0000 | 2.0000 to 4.0000 | |
| 75 | 19.2500 | 12.0000 to 29.7738 | |
| 90 | 40.0000 | 32.0773 to 47.0000 | |
| 95 | 47.0000 | 42.1962 to 52.2310 | |
| 97.5 | 50.0000 | | |

TABLE 6B

| Variable | | Psoriasis_95th_precentile | |
|---|---|---|---|
| | | Psoriasis 95th precentile | |
| Sample size | | 133 | |
| Lowest value | | 0.0000 | |
| Highest value | | 54.0000 | |
| Arithmetic mean | | 9.5940 | |
| 95% CI for the mean | | 7.3625 to 11.8255 | |
| Median | | 4.0000 | |
| 95% CI for the median | | 3.0000 to 6.0000 | |
| Variance | | 169.2581 | |
| Standard deviation | | 13.0099 | |
| Relative standard deviation | | 1.3561 | (135.61%) |
| Standard error of the mean | | 1.1281 | |
| Coefficient of Skewness | | 1.7172 | (P < 0.0001) |
| Coefficient of Kurtosis | | 2.0139 | (P = 0.0026) |
| D'Agostino-Pearson test | | reject Normality | (P < 0.0001) |
| for Normal distribution | | | |
| Percentiles | | 95% Confidence interval | |
| 2.5 | 0.0000 | | |
| 5 | 0.0000 | 0 0000 to 0.0000 | |
| 10 | 0.0000 | 0 0000 to 0.0000 | |
| 25 | 1.0000 | 1.0000 to 2.0000 | |
| 75 | 12.0000 | 8.0000 to 18.0000 | |
| 90 | 36.0000 | 23.0773 to 39.5554 | |
| 95 | 39.8500 | 36.0000 to 47.9521 | |
| 97.5 | 44.3500 | | |

TABLE 7A

| Variable | Non_Psoriasis_90th_precentile | |
|---|---|---|
| | Non- Psoriasis 90th precentile | |
| Sample size | 240 | |
| Lowest value | 0.0000 | |
| Highest value | 44.0000 | |
| Arithmetic mean | 5.7833 | |
| 95% CI for the mean | 4.8519 to 6.7147 | |
| Median | 3.0000 | |
| 95% CI for the median | 2.0000 to 4.0000 | |
| Variance | 53.6516 | |
| Standard deviation | 7.3247 | |
| Relative standard deviation | 1.2665 | (126.65%) |
| Standard error of the mean | 0.4728 | |
| Coefficient of Skewness | 2.1466 | (P < 0.0001) |
| Coefficient of Kurtosis | 5.1163 | (P < 0.0001) |
| D'Agostino-Pearson test | reject Normality | (P < 0.0001) |
| for Normal distribution | | |

| Percentiles | | 95% Confidence interval |
|---|---|---|
| 2.5 | 0.0000 | 0.0000 to 0.0000 |
| 5 | 0.0000 | 0.0000 to 0.0000 |
| 10 | 0.0000 | 0.0000 to 0.0000 |
| 25 | 1.0000 | 1.0000 to 1.0000 |
| 75 | 8.0000 | 6.0000 to 9.0000 |
| 90 | 15.0000 | 12.0000 to 22.0000 |
| 95 | 23.0000 | 18.9920 to 28.0000 |
| 97.5 | 28.0000 | 23.3642 to 32.4280 |

TABLE 7B

| Variable | Non_Psoriasis_95th_precentile | |
|---|---|---|
| | Non- Psoriasis 95th precentile | |
| Sample size | 240 | |
| Lowest value | 0.0000 | |
| Highest value | 26.0000 | |
| Arithmetic mean | 2.9292 | |
| 95% CI for the mean | 2.3872 to 3.4711 | |
| Median | 1.0000 | |
| 95% CI for the median | 1.0000 to 2.0000 | |
| Variance | 18.1665 | |
| Standard deviation | 4.2622 | |
| Relative standard deviation | 1.4551 | (145.51%) |
| Standard error of the mean | 0.2751 | |
| Coefficient of Skewness | 2.3449 | (P < 0.0001) |
| Coefficient of Kurtosis | 6.5236 | (P < 0.0001) |
| D'Agostino-Pearson test | reject Normality | (P < 0.0001) |
| for Normal distribution | | |

| Percentiles | | 95% Confidence interval |
|---|---|---|
| 2.5 | 0.0000 | 0.0000 to 0.0000 |
| 5 | 0.0000 | 0.0000 to 0.0000 |
| 10 | 0.0000 | 0.0000 to 0.0000 |
| 25 | 0.0000 | 0.0000 to 0.0000 |
| 75 | 4.0000 | 3.0000 to 5.0000 |
| 90 | 9.0000 | 6.0405 to 11.0000 |
| 95 | 12.0000 | 10.0000 to 16.0000 |
| 97.5 | 16.0000 | 12.3642 to 21.2839 |

TABLE 8A

| Variable | Psoriasis_90th_precentile_1 |
|---|---|
| | Psoriasis 90th precentile_1 |

Back-transformed after logarithmic transformation.

| Sample size | | 133 | |
|---|---|---|---|
| Lowest value | | 0.1000 | |
| Highest value | | 59.0000 | |
| Geometric mean | | 5.7525 | |
| 95% CI for the mean | | 4.3355 to 7.6326 | |
| Median | | 8.0000 | |
| 95% CI for the median | | 6.0000 to 10.0000 | |
| Coefficient of Skewness | | −0.9815 | (P < 0.0001) |
| Coefficient of Kurtosis | | 0.6943 | (P = 0.1265) |
| D'Agostino-Pearson test | | reject Normality | (P = 0.0001) |
| for Normal distribution | | | |
| Percentiles | | 95% Confidence interval | |
| 2.5 | 0.10000 | | |
| 5 | 0.10000 | 0.10000 to 1.0000 | |
| 10 | 1.0000 | 0.10000 to 1.8956 | |
| 25 | 2.0000 | 2.0000 to 4.0000 | |
| 75 | 19.2452 | 12.0000 to 29.7708 | |
| 90 | 40.0000 | 32.0763 to 47.0000 | |
| 95 | 47.0000 | 42.1489 to 52.1130 | |
| 97.5 | 50.0000 | | |

TABLE 8B

| Variable | Psoriasis_95th_precentile_1 |
|---|---|
| | Psoriasis 95th precentile_1 |

Back-transformed after logarithmic transformation.

| Sample size | | 133 | |
|---|---|---|---|
| Lowest value | | 0.1000 | |
| Highest value | | 54.0000 | |
| Geometric mean | | 2.9541 | |
| 95% CI for the mean | | 2.1402 to 4.0774 | |
| Median | | 4.0000 | |
| 95% CI for the median | | 3.0000 to 6.0000 | |
| Coefficient of Skewness | | −0.5344 | (P = 0.0132) |
| Coefficient of Kurtosis | | −0.5936 | (P = 0.0635) |
| D'Agostino-Pearson test | | reject Normality | (P = 0.0083) |
| for Normal distribution | | | |
| Percentiles | | 95% Confidence interval | |
| 2.5 | 0.10000 | | |
| 5 | 0.10000 | 0.10000 to 0.10000 | |
| 10 | 0.10000 | 0.10000 to 0.10000 | |
| 25 | 1.0000 | 1.0000 to 2.0000 | |
| 75 | 12.0000 | 8.0000 to 18.0000 | |
| 90 | 36.0000 | 23.0758 to 39.5523 | |
| 95 | 39.8484 | 36.0000 to 47.8535 | |
| 97.5 | 44.3436 | | |

TABLE 9A

| Variable | Non_Psoriasis_90th_precentile_1 |
|---|---|
| | Non- Psoriasis 90th precentile_1 |

Back-transformed after logarithmic transformation.

| Sample size | 240 | |
|---|---|---|
| Lowest value | 0.1000 | |
| Highest value | 44.0000 | |
| Geometric mean | 2.2995 | |
| 95% CI for the mean | 1.8657 to 2.8342 | |
| Median | 3.0000 | |
| 95% CI for the median | 2.0000 to 4.0000 | |
| Coefficient of Skewness | −0.6604 | (P = 0.0001) |
| Coefficient of Kurtosis | −0.3565 | (P = 0.2046) |
| D'Agostino-Pearson test | reject Normality | (P = 0.0002) |
| for Normal distribution | | |

| Percentiles | | 95% Confidence interval |
|---|---|---|
| 2.5 | 0.10000 | 0.10000 to 0.10000 |
| 5 | 0.10000 | 0.10000 to 0.10000 |
| 10 | 0.10000 | 0.10000 to 0.10000 |
| 25 | 1.0000 | 1.0000 to 1.0000 |
| 75 | 8.0000 | 6.0000 to 9.0000 |
| 90 | 15.0000 | 12.0000 to 22.0000 |
| 95 | 23.0000 | 18.9656 to 28.0000 |
| 97.5 | 28.0000 | 23.3593 to 32.4152 |

TABLE 9B

| Variable | Non_Psoriasis_95th_precentile_1 |
|---|---|
| | Non- Psoriasis 95th precentile_1 |

Back-transformed after logarithmic transformation.

| Sample size | 240 | |
|---|---|---|
| Lowest value | 0.1000 | |
| Highest value | 26.0000 | |
| Geometric mean | 0.9065 | |
| 95% CI for the mean | 0.7232 to 1.1361 | |
| Median | 1.0000 | |
| 95% CI for the median | 1.0000 to 2.0000 | |
| Coefficient of Skewness | −0.1139 | (P = 0.4626) |
| Coefficient of Kurtosis | −1.4181 | (P < 0.0001) |
| D'Agostino-Pearson test | reject Normality | (P < 0.0001) |
| for Normal distribution | | |

| Percentiles | | 95% Confidence interval |
|---|---|---|
| 2.5 | 0.10000 | 0.10000 to 0.10000 |
| 5 | 0.10000 | 0.10000 to 0.10000 |
| 10 | 0.10000 | 0.10000 to 0.10000 |
| 25 | 0.10000 | 0.10000 to 0.10000 |
| 75 | 4.0000 | 3.0000 to 5.0000 |
| 90 | 9.0000 | 6.0376 to 11.0000 |
| 95 | 12.0000 | 10.0000 to 16.0000 |
| 97.5 | 16.0000 | 12.3550 to 21.0951 |

TABLE 10A
Sample 1

| | |
|---|---|
| Variable | Non_Psoriasis_90th_precentile_1 |
| | Non- Psoriasis 90th precentile_1 |

Sample 2

| | |
|---|---|
| Variable | Psoriasis_90th_precentile_1 |
| | Psoriasis 90th precentile 1 |

Back-transformed after logarithmic transformation.

| | Sample 1 | Sample 2 |
|---|---|---|
| Sample size | 240 | 133 |
| Geometric mean | 2.2995 | 5.7525 |
| 95% CI for the mean | 1.8657 to 2.8342 | 4.3355 to 7.6326 |
| Variance of Logs | 0.5099 | 0.5127 |

| | |
|---|---|
| F-test for equal variances | P = 0.959 |

T-test (assuming equal variances)
Difference on Log-transformed scale

| | |
|---|---|
| Difference | 0.3982 |
| Standard Error | 0.07727 |
| 95% CI of difference | 0.2463 to 0.5502 |
| Test statistic t | 5.154 |
| Degrees of Freedom (DF) | 371 |
| Two-tailed probability | P < 0.0001 |

Back-transformed results

| | |
|---|---|
| Ratio of geometric means | 2.5016 |
| 95% CI of ratio | 1.7631 to 3.5494 |

TABLE 10B
Sample 1

| | |
|---|---|
| Variable | Non_Psoriasis_95th_precentile_1 |
| | Non- Psoriasis 95th precentile_1 |

Sample 2

| | |
|---|---|
| Variable | Psoriasis_95th_precentile_1 |
| | Psoriasis 95th precentile_1 |

Back-transformed after logarithmic transformation.

| | Sample 1 | Sample 2 |
|---|---|---|
| Sample size | 240 | 133 |
| Geometric mean | 0.9065 | 2.9541 |
| 95% CI for the mean | 0.7232 to 1.1361 | 2.1402 to 4.0774 |
| Variance of Logs | 0.5949 | 0.6659 |

| | |
|---|---|
| F-test for equal variances | P = 0.451 |

T-test (assuming equal variances)
Difference on Log-transformed scale

| | |
|---|---|
| Difference | 0.5131 |
| Standard Error | 0.08513 |
| 95% CI of difference | 0.3457 to 0.6805 |
| Test statistic t | 6.027 |
| Degrees of Freedom (DF) | 371 |
| Two-tailed probability | P < 0.0001 |

Back-transformed results

| | |
|---|---|
| Ratio of geometric means | 3.2589 |
| 95% CI of ratio | 2.2166 to 4.7914 |

TABLE 11A
Sample 1

| | |
|---|---|
| Variable | Non_Psoriasis_90th_precentile_1 |
| | Non- Psoriasis 90th precentile_1 |

Sample 2

| | |
|---|---|
| Variable | Psoriasis_90th_precentile_1 |
| | Psoriasis 90th precentile_1 |

| | Sample 1 | Sample 2 |
|---|---|---|
| Sample size | 240 | 133 |
| Lowest value | 0.1000 | 0.1000 |
| Highest value | 44.0000 | 59.0000 |
| Median | 3.0000 | 8.0000 |
| 95% CI for the median | 2.0000 to 4.0000 | 6.0000 to 10.0000 |
| Interquartile range | 1.0000 to 8.0000 | 2.0000 to 19.2500 |

Mann-Whitney test (independent samples)

| | |
|---|---|
| Average rank of first group | 164.3354 |
| Average rank of second group | 227.8985 |
| Mann-Whitney U | 10520.50 |
| Test statistic Z (corrected for ties) | 5.474 |
| Two-tailed probability | P < 0.0001 |

TABLE 11B
Sample 1

| | |
|---|---|
| Variable | Non_Psoriasis_95th_precentile_1 |
| | Non- Psoriasis 95th precentile_1 |

Sample 2

| | |
|---|---|
| Variable | Psoriasis_95th_precentile_1 |
| | Psoriasis 95th precentile_1 |

| | Sample 1 | Sample 2 |
|---|---|---|
| Sample size | 240 | 133 |
| Lowest value | 0.1000 | 0.1000 |
| Highest value | 26.0000 | 54.0000 |
| Median | 1.0000 | 4.0000 |
| 95% CI for the median | 1.0000 to 2.0000 | 3.0000 to 6.0000 |
| Interquartile range | 0.1000 to 4.0000 | 1.0000 to 12.0000 |

Mann-Whitney test (independent samples)

| | |
|---|---|
| Average rank of first group | 163.5479 |
| Average rank of second group | 229.3195 |
| Mann-Whitney U | 10331.50 |
| Test statistic Z (corrected for ties) | 5.726 |
| Two-tailed probability | P < 0.0001 |

TABLE 12A

| Variable | Psoriasis_Test |
|---|---|
| Classification variable | Diagnosis_1_Psoriasis_0_Non_Psoriasis_{—} |
| | Diagnosis(1_Psoriasis 0_Non-Psoriasis) |
| Sample size | 373 |
| Positive group ^{a} | 133 (35.66%) |
| Negative group ^{b} | 240 (64.34%) |

^{a} Diagnosis_1_Psoriasis_0_Non_Psoriasis_ = 1
^{b} Diagnosis_1_Psoriasis_0_Non_Psoriasis_ = 0

| | |
|---|---|
| Disease prevalence (%) | unknown |

Area under the ROC curve (AUC)

| | |
|---|---|
| Area under the ROC curve (AUC) | 0.670 |
| Standard Error ^{a} | 0.0297 |
| 95% Confidence interval ^{b} | 0.620 to 0.718 |
| z statistic | 5.742 |
| Significance level P (Area = 0.5) | <0.0001 |

^{a} DeLong et al., 1988
^{b} Binomial exact

Youden index

| | |
|---|---|
| Youden index J | 0.2582 |
| 95% Confidence interval ^{a} | 0.1476 to 0.3283 |
| Associated criterion | >5 |
| 95% Confidence interval ^{a} | >1 to >8 |
| Sensitivity | 61.65 |
| Specificity | 64.17 |

^{a} BCₐ bootstrap confidence interval (1000 iterations; random number seed: 978).

TABLE 12B

| Variable | Psoriasis_Test |
|---|---|
| Classification variable | Diagnosis_1_Psoriasis_0_Non_Psoriasis_{—} |
| | Diagnosis(1_Psoriasis 0_Non-Psoriasis) |
| Sample size | 373 |
| Positive group ^{a} | 133 (35.66%) |
| Negative group ^{b} | 240 (64.34%) |

^{a} Diagnosis_1_Psoriasis_0_Non_Psoriasis_ = 1
^{b} Diagnosis_1_Psoriasis_0_Non_Psoriasis_ = 0

| | |
|---|---|
| Disease prevalence (%) | unknown |

Area under the ROC curve (AUC)

| | |
|---|---|
| Area under the ROC curve (AUC) | 0.676 |
| Standard Error ^{a} | 0.0293 |
| 95% Confidence interval ^{b} | 0.626 to 0.724 |
| z statistic | 6.028 |
| Significance level P (Area = 0.5) | <0.0001 |

^{a} DeLong et al., 1988
^{b} Binomial exact

Youden index

| | |
|---|---|
| Youden index J | 0.2610 |
| 95% Confidence interval ^{a} | 0.1600 to 0.3315 |
| Associated criterion | >6 |
| 95% Confidence interval ^{a} | >2 to >17 |
| Sensitivity | 39.85 |
| Specificity | 86.25 |

^{a} BCₐ bootstrap confidence interval (1000 iterations; random number seed: 978).

TABLE 13A
Performance Metrics in Predicting Psoriasis Status from Number
of Positive Foods Using 90th Percentile of ELISA Signal to
determine Positive

| | No. of | | | | | |
|---|---|---|---|---|---|---|
| | Positive | | | | | Overall |
| | Foods | | | Positive | Negative | Percent |
| | as | Sensi- | Speci- | Predictive | Predictive | Agree- |
| Sex | Cutoff | tivity | ficity | Value | Value | ment |
| FEMALE | 1 | 0.88 | 0.27 | 0.40 | 0.80 | 0.49 |
| | 2 | 0.74 | 0.45 | 0.42 | 0.76 | 0.55 |
| | 3 | 0.67 | 0.56 | 0.46 | 0.76 | 0.60 |
| | 4 | 0.59 | 0.64 | 0.47 | 0.74 | 0.62 |
| | 5 | 0.50 | 0.70 | 0.48 | 0.72 | 0.63 |
| | 6 | 0.40 | 0.76 | 0.48 | 0.70 | 0.64 |
| | 7 | 0.30 | 0.81 | 0.48 | 0.68 | 0.63 |
| | 8 | 0.26 | 0.84 | 0.46 | 0.67 | 0.63 |
| | 9 | 0.22 | 0.85 | 0.45 | 0.67 | 0.63 |
| | 10 | 0.20 | 0.87 | 0.47 | 0.66 | 0.63 |
| | 11 | 0.18 | 0.88 | 0.47 | 0.66 | 0.63 |
| | 12 | 0.16 | 0.89 | 0.46 | 0.66 | 0.63 |
| | 13 | 0.15 | 0.91 | 0.50 | 0.66 | 0.64 |
| | 14 | 0.15 | 0.92 | 0.53 | 0.66 | 0.65 |
| | 15 | 0.15 | 0.93 | 0.55 | 0.67 | 0.66 |
| | 16 | 0.14 | 0.95 | 0.60 | 0.67 | 0.66 |
| | 17 | 0.13 | 0.96 | 0.64 | 0.67 | 0.66 |
| | 18 | 0.10 | 0.97 | 0.67 | 0.66 | 0.66 |
| | 19 | 0.09 | 0.98 | 0.71 | 0.66 | 0.66 |
| | 20 | 0.08 | 0.99 | 0.80 | 0.66 | 0.67 |
| | 21 | 0.08 | 1.00 | 1.00 | 0.66 | 0.67 |
| | 22 | 0.07 | 1.00 | 1.00 | 0.66 | 0.67 |
| | 23 | 0.07 | 1.00 | 1.00 | 0.66 | 0.67 |
| | 24 | 0.06 | 1.00 | 1.00 | 0.66 | 0.67 |
| | 25 | 0.05 | 1.00 | 1.00 | 0.66 | 0.67 |
| | 26 | 0.05 | 1.00 | 1.00 | 0.66 | 0.66 |
| | 27 | 0.03 | 1.00 | 1.00 | 0.65 | 0.66 |
| | 28 | 0.03 | 1.00 | 1.00 | 0.65 | 0.66 |
| | 29 | 0.03 | 1.00 | 1.00 | 0.65 | 0.66 |
| | 30 | 0.02 | 1.00 | 1.00 | 0.65 | 0.65 |
| | 31 | 0.02 | 1.00 | 1.00 | 0.65 | 0.65 |
| | 32 | 0.02 | 1.00 | 1.00 | 0.65 | 0.65 |
| | 33 | 0.02 | 1.00 | 1.00 | 0.65 | 0.65 |
| | 34 | 0.02 | 1.00 | 1.00 | 0.65 | 0.65 |
| | 35 | 0.00 | 1.00 | 1.00 | 0.65 | 0.65 |
| | 36 | 0.00 | 1.00 | — | 0.65 | 0.65 |
| | 37 | 0.00 | 1.00 | — | 0.65 | 0.65 |
| | 38 | 0.00 | 1.00 | — | 0.65 | 0.65 |
| | 39 | 0.00 | 1.00 | — | 0.65 | 0.65 |
| | 40 | 0.00 | 1.00 | — | 0.65 | 0.65 |
| | 41 | 0.00 | 1.00 | — | 0.65 | 0.65 |
| | 42 | 0.00 | 1.00 | — | 0.65 | 0.65 |
| | 43 | 0.00 | 1.00 | — | 0.65 | 0.65 |
| | 44 | 0.00 | 1.00 | — | 0.65 | 0.65 |
| | 45 | 0.00 | 1.00 | — | 0.65 | 0.65 |
| | 46 | 0.00 | 1.00 | — | 0.65 | 0.65 |
| | 47 | 0.00 | 1.00 | — | 0.65 | 0.65 |
| | 48 | 0.00 | 1.00 | — | 0.65 | 0.65 |
| | 49 | 0.00 | 1.00 | — | 0.65 | 0.65 |
| | 50 | 0.00 | 1.00 | — | 0.65 | 0.65 |
| | 51 | 0.00 | 1.00 | — | 0.65 | 0.65 |
| | 52 | 0.00 | 1.00 | — | 0.65 | 0.65 |
| | 53 | 0.00 | 1.00 | — | 0.65 | 0.65 |
| | 54 | 0.00 | 1.00 | — | 0.65 | 0.65 |
| | 55 | 0.00 | 1.00 | — | 0.65 | 0.65 |
| | 56 | 0.00 | 1.00 | — | 0.65 | 0.65 |
| | 57 | 0.00 | 1.00 | — | 0.65 | 0.65 |
| | 58 | 0.00 | 1.00 | — | 0.65 | 0.65 |
| | 59 | 0.00 | 1.00 | — | 0.65 | 0.65 |

TABLE 13B

| | No. of | | | | | |
|---|---|---|---|---|---|---|
| | Positive | | | | | Overall |
| | Foods | | | Positive | Negative | Percent |
| | as | Sensi- | Speci- | Predictive | Predictive | Agree- |
| Sex | Cutoff | tivity | ficity | Value | Value | ment |
| MALE | 1 | 0.93 | 0.16 | 0.38 | 0.80 | 0.43 |
| | 2 | 0.81 | 0.32 | 0.40 | 0.75 | 0.49 |
| | 3 | 0.71 | 0.44 | 0.41 | 0.73 | 0.53 |
| | 4 | 0.64 | 0.52 | 0.43 | 0.73 | 0.56 |
| | 5 | 0.58 | 0.59 | 0.44 | 0.71 | 0.58 |
| | 6 | 0.54 | 0.65 | 0.47 | 0.72 | 0.62 |
| | 7 | 0.51 | 0.71 | 0.50 | 0.72 | 0.64 |
| | 8 | 0.49 | 0.77 | 0.54 | 0.73 | 0.67 |
| | 9 | 0.45 | 0.81 | 0.57 | 0.73 | 0.68 |
| | 10 | 0.43 | 0.85 | 0.61 | 0.73 | 0.70 |
| | 11 | 0.40 | 0.88 | 0.65 | 0.72 | 0.71 |
| | 12 | 0.39 | 0.90 | 0.68 | 0.72 | 0.71 |
| | 13 | 0.37 | 0.91 | 0.70 | 0.72 | 0.72 |
| | 14 | 0.36 | 0.92 | 0.71 | 0.72 | 0.72 |
| | 15 | 0.35 | 0.93 | 0.73 | 0.72 | 0.72 |
| | 16 | 0.34 | 0.93 | 0.74 | 0.72 | 0.72 |
| | 17 | 0.33 | 0.94 | 0.75 | 0.72 | 0.72 |
| | 18 | 0.33 | 0.95 | 0.76 | 0.71 | 0.72 |
| | 19 | 0.31 | 0.95 | 0.77 | 0.71 | 0.72 |
| | 20 | 0.28 | 0.96 | 0.79 | 0.71 | 0.72 |
| | 21 | 0.27 | 0.96 | 0.80 | 0.70 | 0.71 |
| | 22 | 0.26 | 0.96 | 0.81 | 0.70 | 0.71 |
| | 23 | 0.25 | 0.97 | 0.82 | 0.70 | 0.71 |
| | 24 | 0.24 | 0.97 | 0.85 | 0.70 | 0.71 |
| | 25 | 0.23 | 0.99 | 0.88 | 0.69 | 0.71 |
| | 26 | 0.21 | 0.99 | 0.89 | 0.69 | 0.71 |
| | 27 | 0.20 | 0.99 | 0.90 | 0.69 | 0.71 |
| | 28 | 0.20 | 0.99 | 0.90 | 0.69 | 0.70 |
| | 29 | 0.19 | 0.99 | 0.90 | 0.69 | 0.70 |
| | 30 | 0.18 | 0.99 | 0.90 | 0.69 | 0.70 |
| | 31 | 0.17 | 0.99 | 0.91 | 0.68 | 0.70 |
| | 32 | 0.16 | 0.99 | 0.92 | 0.68 | 0.69 |
| | 33 | 0.15 | 1.00 | 1.00 | 0.68 | 0.69 |
| | 34 | 0.14 | 1.00 | 1.00 | 0.68 | 0.69 |
| | 35 | 0.13 | 1.00 | 1.00 | 0.67 | 0.68 |
| | 36 | 0.11 | 1.00 | 1.00 | 0.67 | 0.68 |
| | 37 | 0.10 | 1.00 | 1.00 | 0.66 | 0.68 |
| | 38 | 0.07 | 1.00 | 1.00 | 0.66 | 0.67 |
| | 39 | 0.05 | 1.00 | 1.00 | 0.65 | 0.66 |
| | 40 | 0.02 | 1.00 | 1.00 | 0.65 | 0.65 |
| | 41 | 0.02 | 1.00 | 1.00 | 0.65 | 0.65 |
| | 42 | 0.02 | 1.00 | 1.00 | 0.65 | 0.65 |
| | 43 | 0.02 | 1.00 | 1.00 | 0.65 | 0.65 |
| | 44 | 0.02 | 1.00 | 1.00 | 0.65 | 0.65 |
| | 45 | 0.02 | 1.00 | 1.00 | 0.65 | 0.65 |
| | 46 | 0.02 | 1.00 | 1.00 | 0.65 | 0.65 |
| | 47 | 0.02 | 1.00 | 1.00 | 0.65 | 0.65 |
| | 48 | 0.00 | 1.00 | 1.00 | 0.64 | 0.64 |
| | 49 | 0.00 | 1.00 | — | 0.64 | 0.64 |
| | 50 | 0.00 | 1.00 | — | 0.64 | 0.64 |
| | 51 | 0.00 | 1.00 | — | 0.64 | 0.64 |
| | 52 | 0.00 | 1.00 | — | 0.64 | 0.64 |
| | 53 | 0.00 | 1.00 | — | 0.64 | 0.64 |
| | 54 | 0.00 | 1.00 | — | 0.64 | 0.64 |
| | 55 | 0.00 | 1.00 | — | 0.64 | 0.64 |
| | 56 | 0.00 | 1.00 | — | 0.64 | 0.64 |
| | 57 | 0.00 | 1.00 | — | 0.64 | 0.64 |
| | 58 | 0.00 | 1.00 | — | 0.64 | 0.64 |
| | 59 | 0.00 | 1.00 | — | 0.64 | 0.64 |

TABLE 14A
Performance Metrics in Predicting Psoriasis Status
from Number of Positive Foods Using 95th Percentile
of ELISA Signal to determine Positive

| | No. of | | | | | |
|---|---|---|---|---|---|---|
| | Positive | | | | | Overall |
| | Foods | | | Positive | Negative | Percent |
| | as | Sensi- | Speci- | Predictive | Predictive | Agree- |
| Sex | Cutoff | tivity | ficity | Value | Value | ment |
| FEMALE | 1 | 0.75 | 0.44 | 0.42 | 0.76 | 0.55 |
| | 2 | 0.61 | 0.62 | 0.47 | 0.74 | 0.62 |
| | 3 | 0.48 | 0.73 | 0.49 | 0.72 | 0.64 |
| | 4 | 0.36 | 0.81 | 0.50 | 0.70 | 0.65 |
| | 5 | 0.26 | 0.85 | 0.48 | 0.68 | 0.64 |
| | 6 | 0.22 | 0.87 | 0.47 | 0.67 | 0.64 |
| | 7 | 0.19 | 0.89 | 0.50 | 0.67 | 0.64 |
| | 8 | 0.17 | 0.91 | 0.50 | 0.67 | 0.65 |
| | 9 | 0.15 | 0.93 | 0.54 | 0.67 | 0.65 |
| | 10 | 0.14 | 0.95 | 0.57 | 0.67 | 0.66 |
| | 11 | 0.13 | 0.96 | 0.64 | 0.67 | 0.66 |
| | 12 | 0.12 | 0.97 | 0.75 | 0.67 | 0.67 |
| | 13 | 0.12 | 0.99 | 0.83 | 0.67 | 0.68 |
| | 14 | 0.11 | 1.00 | 1.00 | 0.67 | 0.68 |
| | 15 | 0.09 | 1.00 | 1.00 | 0.67 | 0.68 |
| | 16 | 0.08 | 1.00 | 1.00 | 0.66 | 0.67 |
| | 17 | 0.08 | 1.00 | 1.00 | 0.66 | 0.67 |
| | 18 | 0.08 | 1.00 | 1.00 | 0.66 | 0.67 |
| | 19 | 0.08 | 1.00 | 1.00 | 0.66 | 0.67 |
| | 20 | 0.08 | 1.00 | 1.00 | 0.66 | 0.67 |
| | 21 | 0.07 | 1.00 | 1.00 | 0.66 | 0.67 |
| | 22 | 0.05 | 1.00 | 1.00 | 0.66 | 0.66 |
| | 23 | 0.05 | 1.00 | 1.00 | 0.66 | 0.66 |
| | 24 | 0.04 | 1.00 | 1.00 | 0.66 | 0.66 |
| | 25 | 0.03 | 1.00 | 1.00 | 0.65 | 0.66 |
| | 26 | 0.03 | 1.00 | 1.00 | 0.65 | 0.66 |
| | 27 | 0.02 | 1.00 | 1.00 | 0.65 | 0.65 |
| | 28 | 0.02 | 1.00 | 1.00 | 0.65 | 0.65 |
| | 29 | 0.02 | 1.00 | 1.00 | 0.65 | 0.65 |
| | 30 | 0.02 | 1.00 | 1.00 | 0.65 | 0.65 |
| | 31 | 0.02 | 1.00 | 1.00 | 0.65 | 0.65 |
| | 32 | 0.00 | 1.00 | 1.00 | 0.65 | 0.65 |
| | 33 | 0.00 | 1.00 | 1.00 | 0.65 | 0.65 |
| | 34 | 0.00 | 1.00 | 1.00 | 0.65 | 0.65 |
| | 35 | 0.00 | 1.00 | 1.00 | 0.65 | 0.65 |
| | 36 | 0.00 | 1.00 | — | 0.65 | 0.65 |
| | 37 | 0.00 | 1.00 | — | 0.65 | 0.65 |
| | 38 | 0.00 | 1.00 | — | 0.65 | 0.65 |
| | 39 | 0.00 | 1.00 | — | 0.65 | 0.65 |
| | 40 | 0.00 | 1.00 | — | 0.65 | 0.65 |
| | 41 | 0.00 | 1.00 | — | 0.65 | 0.65 |
| | 42 | 0.00 | 1.00 | — | 0.65 | 0.65 |
| | 43 | 0.00 | 1.00 | — | 0.65 | 0.65 |
| | 44 | 0.00 | 1.00 | — | 0.65 | 0.65 |
| | 45 | 0.00 | 1.00 | — | 0.65 | 0.65 |
| | 46 | 0.00 | 1.00 | — | 0.65 | 0.65 |
| | 47 | 0.00 | 1.00 | — | 0.65 | 0.65 |
| | 48 | 0.00 | 1.00 | — | 0.65 | 0.65 |
| | 49 | 0.00 | 1.00 | — | 0.65 | 0.65 |
| | 50 | 0.00 | 1.00 | — | 0.65 | 0.65 |
| | 51 | 0.00 | 1.00 | — | 0.65 | 0.65 |
| | 52 | 0.00 | 1.00 | — | 0.65 | 0.65 |
| | 53 | 0.00 | 1.00 | — | 0.65 | 0.65 |
| | 54 | 0.00 | 1.00 | — | 0.65 | 0.65 |
| | 55 | 0.00 | 1.00 | — | 0.65 | 0.65 |
| | 56 | 0.00 | 1.00 | — | 0.65 | 0.65 |
| | 57 | 0.00 | 1.00 | — | 0.65 | 0.65 |
| | 58 | 0.00 | 1.00 | — | 0.65 | 0.65 |
| | 59 | 0.00 | 1.00 | — | 0.65 | 0.65 |

TABLE 14B

| | No. of | | | | | |
|---|---|---|---|---|---|---|
| | Positive | | | | | Overall |
| | Foods | | | Positive | Negative | Percent |
| | as | Sensi- | Speci- | Predictive | Predictive | Agree- |
| Sex | Cutoff | tivity | ficity | Value | Value | ment |
| MALE | 1 | 0.86 | 0.29 | 0.40 | 0.78 | 0.49 |
| | 2 | 0.70 | 0.49 | 0.43 | 0.75 | 0.57 |
| | 3 | 0.57 | 0.61 | 0.45 | 0.72 | 0.60 |
| | 4 | 0.53 | 0.72 | 0.51 | 0.73 | 0.65 |
| | 5 | 0.50 | 0.80 | 0.58 | 0.74 | 0.69 |
| | 6 | 0.47 | 0.86 | 0.65 | 0.74 | 0.72 |
| | 7 | 0.44 | 0.89 | 0.69 | 0.74 | 0.73 |
| | 8 | 0.39 | 0.92 | 0.73 | 0.73 | 0.73 |
| | 9 | 0.36 | 0.93 | 0.75 | 0.72 | 0.73 |
| | 10 | 0.34 | 0.95 | 0.77 | 0.72 | 0.73 |
| | 11 | 0.32 | 0.95 | 0.79 | 0.72 | 0.73 |
| | 12 | 0.31 | 0.96 | 0.81 | 0.71 | 0.73 |
| | 13 | 0.30 | 0.97 | 0.85 | 0.71 | 0.73 |
| | 14 | 0.28 | 0.97 | 0.86 | 0.71 | 0.73 |
| | 15 | 0.27 | 0.98 | 0.88 | 0.70 | 0.72 |
| | 16 | 0.26 | 0.99 | 0.91 | 0.70 | 0.72 |
| | 17 | 0.24 | 0.99 | 0.91 | 0.70 | 0.72 |
| | 18 | 0.22 | 0.99 | 0.91 | 0.69 | 0.71 |
| | 19 | 0.21 | 0.99 | 0.91 | 0.69 | 0.71 |
| | 20 | 0.19 | 0.99 | 0.91 | 0.69 | 0.71 |
| | 21 | 0.18 | 0.99 | 0.92 | 0.69 | 0.70 |
| | 22 | 0.18 | 1.00 | 1.00 | 0.68 | 0.70 |
| | 23 | 0.17 | 1.00 | 1.00 | 0.68 | 0.70 |
| | 24 | 0.16 | 1.00 | 1.00 | 0.68 | 0.70 |
| | 25 | 0.15 | 1.00 | 1.00 | 0.68 | 0.69 |
| | 26 | 0.14 | 1.00 | 1.00 | 0.68 | 0.69 |
| | 27 | 0.13 | 1.00 | 1.00 | 0.67 | 0.69 |
| | 28 | 0.12 | 1.00 | 1.00 | 0.67 | 0.68 |
| | 29 | 0.11 | 1.00 | 1.00 | 0.67 | 0.68 |
| | 30 | 0.09 | 1.00 | 1.00 | 0.66 | 0.68 |
| | 31 | 0.07 | 1.00 | 1.00 | 0.66 | 0.67 |
| | 32 | 0.05 | 1.00 | 1.00 | 0.65 | 0.66 |
| | 33 | 0.03 | 1.00 | 1.00 | 0.65 | 0.66 |
| | 34 | 0.02 | 1.00 | 1.00 | 0.65 | 0.65 |
| | 35 | 0.02 | 1.00 | 1.00 | 0.65 | 0.65 |
| | 36 | 0.02 | 1.00 | 1.00 | 0.65 | 0.65 |
| | 37 | 0.02 | 1.00 | 1.00 | 0.65 | 0.65 |
| | 38 | 0.02 | 1.00 | 1.00 | 0.65 | 0.65 |
| | 39 | 0.02 | 1.00 | 1.00 | 0.65 | 0.65 |
| | 40 | 0.02 | 1.00 | 1.00 | 0.65 | 0.65 |
| | 41 | 0.02 | 1.00 | 1.00 | 0.65 | 0.65 |
| | 42 | 0.02 | 1.00 | 1.00 | 0.65 | 0.65 |
| | 43 | 0.00 | 1.00 | 1.00 | 0.64 | 0.65 |
| | 44 | 0.00 | 1.00 | 1.00 | 0.64 | 0.64 |
| | 45 | 0.00 | 1.00 | 1.00 | 0.64 | 0.64 |
| | 46 | 0.00 | 1.00 | 1.00 | 0.64 | 0.64 |
| | 47 | 0.00 | 1.00 | 1.00 | 0.64 | 0.64 |
| | 48 | 0.00 | 1.00 | 1.00 | 0.64 | 0.64 |
| | 49 | 0.00 | 1.00 | — | 0.64 | 0.64 |
| | 50 | 0.00 | 1.00 | — | 0.64 | 0.64 |
| | 51 | 0.00 | 1.00 | — | 0.64 | 0.64 |
| | 52 | 0.00 | 1.00 | — | 0.64 | 0.64 |
| | 53 | 0.00 | 1.00 | — | 0.64 | 0.64 |
| | 54 | 0.00 | 1.00 | — | 0.64 | 0.64 |
| | 55 | 0.00 | 1.00 | — | 0.64 | 0.64 |
| | 56 | 0.00 | 1.00 | — | 0.64 | 0.64 |
| | 57 | 0.00 | 1.00 | — | 0.64 | 0.64 |
| | 58 | 0.00 | 1.00 | — | 0.64 | 0.64 |
| | 59 | 0.00 | 1.00 | — | 0.64 | 0.64 |

1. A psoriasis test kit panel consisting essentially of:
a plurality of distinct psoriasis trigger food preparations immobilized to an individually addressable solid carrier;
wherein the plurality of distinct psoriasis trigger food preparations each have a raw p-value of ≤0.07 or a false discovery rate (FDR) multiplicity adjusted p-value of ≤0.10.

2. (canceled)

3. The test kit panel of claim 1, wherein the plurality of distinct psoriasis trigger food preparations includes at least two food preparations selected from the group consisting of chocolate, grapefruit, honey, malt, rye, baker's yeast, brewer's yeast, broccoli, cola nut, tobacco, mustard, green pepper, buck wheat, avocado, cane sugar, cantaloupe, garlic, cucumber, cauliflower, sunflower seed, lemon, strawberry, eggplant, wheat, olive, halibut, cabbage, orange, rice, safflower, tomato, almond, oat, barley, peach, grape, potato, spinach, sole, butter, goat's milk, onion, egg, sweet potato, cow's milk, and cheddar cheese.

4. The test kit panel of claim 3, wherein the plurality comprises at least eight distinct psoriasis trigger food preparations.

5. The test kit panel of claim 3, wherein the plurality comprises at least twelve distinct psoriasis trigger food preparations.

6. The test kit panel of claim 1, wherein the plurality of distinct psoriasis trigger food preparations each have a raw p-value of ≤0.05 or a false discovery rate (FDR) multiplicity adjusted p-value of ≤0.07.

7.-9. (canceled)

10. The test kit panel of claim 1, wherein the FDR multiplicity adjusted p-value is adjusted for at least one of age or gender.

11.-13. (canceled)

14. The test kit panel of claim 1, wherein at least 50% of the plurality of distinct psoriasis trigger food preparations, when adjusted for a single gender, have a raw p-value of ≤0.07 or a false discovery rate (FDR) multiplicity adjusted p-value of ≤0.10.

15.-19. (canceled)

20. The test kit panel of claim 1, wherein the plurality of distinct psoriasis trigger food preparations is a crude filtered aqueous extract or a processed aqueous extract.

21.-23. (canceled)

24. The test kit panel of claim 1, wherein the solid carrier is selected from the group consisting of a well of a microwell plate, a dipstick, a membrane-bound array, multiwall plate, a bead, an electrical sensor, a chemical sensor, a microchip and an adsorptive film.

25. (canceled)

26. A method comprising:
contacting a test kit panel consisting essentially of a plurality of distinct psoriasis trigger food preparations with a bodily fluid of a patient that is diagnosed with or suspected of having psoriasis, wherein the contacting is performed under conditions that allow at least a portion of an immunoglobulin from the bodily fluid to bind to the at least one component of the plurality of distinct psoriasis trigger food preparations;
measuring the immunoglobulin bound to the at least one component of the plurality of distinct psoriasis trigger food preparations to obtain a signal;
and
updating or generating a report using the signal.

27.-29. (canceled)

30. The method of claim 26, wherein the plurality of distinct psoriasis trigger food preparations includes at least two food preparations selected from the group consisting of chocolate, grapefruit, honey, malt, rye, baker's yeast, brewer's yeast, broccoli, cola nut, tobacco, mustard, green pepper, buck wheat, avocado, cane sugar, cantaloupe, garlic, cucumber, cauliflower, sunflower seed, lemon, strawberry, eggplant, wheat, olive, halibut, cabbage, orange, rice, safflower, tomato, almond, oat, barley, peach, grape, potato, spinach, sole, butter, goat's milk, onion, egg, sweet potato, cow's milk, and cheddar cheese.

31. (canceled)

32. The method of claim 26, wherein the plurality of distinct psoriasis trigger food preparations each have a raw p-value of ≤0.07 or a false discovery rate (FDR) multiplicity adjusted p-value of ≤0.10.

33. (canceled)

34. The method of claim 26, wherein the plurality of distinct psoriasis trigger food preparations each have a raw p-value of ≤0.05 or a false discovery rate (FDR) multiplicity adjusted p-value of ≤0.07.

35.-45. (canceled)

46. A method of generating a test for patients diagnosed with or suspected of having psoriasis, comprising:
obtaining test results for a plurality of distinct food preparations, wherein the test results are based on bodily fluids of patients diagnosed with or suspected of having psoriasis, and bodily fluids of a control group not diagnosed with or suspected of having psoriasis; and
stratifying the test results by gender group for each of the distinct food preparations;
assigning for a predetermined percentile rank a different cutoff value for each gender group for each of the distinct food preparations;
selecting a plurality of distinct psoriasis trigger food preparations that each have a raw p-value of ≤0.07 or a FDR multiplicity adjusted p-value of ≤0.10; and
generating a test comprising selected distinct psoriasis trigger food preparations in a patient diagnosed with or suspected of having psoriasis.

47. (canceled)

48. The method of claim 46, wherein the plurality of distinct psoriasis trigger food preparations includes at least two food preparations selected from the group consisting of chocolate, grapefruit, honey, malt, rye, baker's yeast, brewer's yeast, broccoli, cola nut, tobacco, mustard, green pepper, buck wheat, avocado, cane sugar, cantaloupe, garlic, cucumber, cauliflower, sunflower seed, lemon, strawberry, eggplant, wheat, olive, halibut, cabbage, orange, rice, safflower, tomato, almond, oat, barley, peach, grape, potato, spinach, sole, butter, goat's milk, onion, egg, sweet potato, cow's milk, and cheddar cheese.

49.-55. (canceled)

56. The method of claim 46, wherein the plurality of distinct psoriasis trigger food preparations each have a raw p-value of ≤0.05 or a FDR multiplicity adjusted p-value of ≤0.08.

57.-61. (canceled)

62. The method of claim 46, wherein the predetermined percentile rank is at least a 90^{th} percentile rank.

63. (canceled)

64. The method of claim 46, wherein the cutoff value for the gender groups has a difference of at least 10% (abs).

65. (canceled)

66. The method of claim 46, further comprising a step of normalizing each test result to each patient's total IgG.

67. (canceled)

68. The method of claim 46, further comprising a step of normalizing the result to the global mean of the patient's food specific IgG results.

69.-101. (canceled)
